SEQUENCE LISTING (33 sequences)

SEQ ID NO: 1 - protein, 227 aa, synthetic construct
  VARIANT 1: note = D or absent
  VARIANT 2: note = K or absent
  VARIANT 3: note = T or absent
  VARIANT 4: note = H or absent
  VARIANT 5: note = T or absent
  VARIANT 6: note = C or absent
  VARIANT 226: note = G or absent
  VARIANT 227: note = K or absent
XXXXXXPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPXX

SEQ ID NO: 2 - protein, 227 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 3 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 4 - protein, 225 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 5 - protein, 226 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 6 - protein, 225 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 7 - protein, 224 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 8 - protein, 225 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 9 - protein, 224 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 10 - protein, 223 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 11 - protein, 223 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 12 - protein, 222 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 13 - protein, 221 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 14 - protein, 221 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 15 - protein, 220 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 16 - protein, 219 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 17 - protein, 224 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 18 - protein, 223 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 19 - protein, 222 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 20 - protein, 221 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 21 - protein, 222 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 22 - protein, 220 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 23 - protein, 216 aa, synthetic construct
QSALTQPASVSGSPGQSITISCTGTGSDVGSYNLVSWYQQHPGKAPKLMIYGDSERPSGVSNRFSGSKSGNTASLTISGLQAEDEADYYCSSYAGSGIYVFGTGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 24 - protein, 445 aa, synthetic construct
EVQLLESGGGLVQPGGSLRLSCAASGFTFSTYAMGWVRQAPGKGLEWVSSIGASGSQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 219 aa, synthetic construct
DIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 444 aa, synthetic construct
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 214 aa, synthetic construct
DIQMTQSPSSLSASVGDRVTITCKASDHINNWLAWYQQKPGQAPRLLISGATSLETGVPSRFSGSGTGKDYTLTISSLQPEDFATYYCQQYWSTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 444 aa, synthetic construct
QVQLVQSGAELKKPGASVKLSCKASGYTFTSYGISWVKQATGQGLEWIGEIYPRSGNTYYNEKFKGRATLTADKSTSTAYMELRSLRSEDSAVYFCARSTTVRPPGIWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 214 aa, synthetic construct
SYVLTQSPSVSVAPGQTARITCGGNNIGSKSVHWYQQKPGQAPVLVVYDDSDRPSGIPERFSASNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 30 - protein, 450 aa, synthetic construct
QLLLQESGPGLVKPSETLSLTCTVSGGSLSSSFSYWVWIRQPPGKGLEWIGTIYYSGNTYYNPSLKSRLTISVDTSKNHFSLKLSSVTAADTAVYYCARRAGILTGYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 15 aa, synthetic construct
EPKSCDKTHTCPPCP

SEQ ID NO: 32 - protein, 509 aa, Homo sapiens
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 33 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCPNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

(19) United States
(12) Patent Application Publication
(10) Pub. No.: US 2026/0008854 A1
(43) Pub. Date: Jan. 8, 2026

(54) METHODS FOR VACCINATION OF A SUBJECT TREATED WITH AN FCRN ANTAGONIST

(71) Applicant: argenx BV, Ghent (BE)

(72) Inventors: Sophie Steeland, Ghent (BE); Benjamin Van Hoorick, Ghent (BE); Antonio Guglietta, Barcelona (ES)

(21) Appl. No.: 19/328,494

(22) Filed: Sep. 15, 2025

Related U.S. Application Data
(63) Continuation of application No. PCT/IB2024/000120, filed on Mar. 14, 2024.
(60) Provisional application No. 63/490,083, filed on Mar. 14, 2023.

(51) Int. Cl. C07K 16/28; A61K 39/00; A61K 45/06; A61P 37/06

(52) U.S. Cl. CPC C07K 16/283; A61K 45/06; A61P 37/06; A61K 2039/505; A61K 2039/545; C07K 2317/76

(57) ABSTRACT

The present disclosure provides methods for vaccination of subjects treated with a human neonatal Fc receptor (FcRn) antagonist. The present disclosure also provides methods of administering an FcRn antagonist to a subject that has recently received a vaccine or will soon receive a vaccine. In some embodiments, the FcRn antagonist is efgartigimod.

### CROSS REFERENCE TO RELATED APPLICATIONS

This application is a Continuation of International Patent Application No. PCT/IB2024/000120, filed Mar. 14, 2024, which claims the benefit of and priority to U.S. Provisional Patent Application No. 63/490,083, filed Mar. 14, 2023, the entire disclosure of each of which are hereby incorporated by reference.

### REFERENCE TO SEQUENCE LISTING

This application contains a sequence listing which has been submitted electronically in ST.26 format and is hereby incorporated by reference in its entirety (said ST.26 copy created on Mar. 5, 2024, is named “404373-T2211WO_SL.xml” and is 40,720 bytes in size).

### FIELD

The present disclosure relates to methods for vaccination of subjects treated with an antagonist of human neonatal Fc receptor (FcRn), which in certain embodiments, is efgartigimod. The present disclosure also relates to methods of administering an FcRn antagonist, which in certain embodiments is efgartigimod, to a subject that has recently received a vaccine or will soon receive a vaccine.

### BACKGROUND

Immunoglobulin G (IgG)-mediated autoimmune diseases, such as Pemphigus and generalized myasthenia gravis (gMG), represent a continual and increasing health burden. Pemphigus is characterized by the presence of autoantibodies targeting epidermal desmoglein (Dsg)-3 and/or Dsg-1 and comprises two main subtypes, Pemphigus vulgaris (PV) and Pemphigus foliaceus (PF). Clinical manifestations include mucosal and skin lesions that may become life-threatening (primarily due to secondary infections) if left untreated. gMG is a rare chronic disease that causes debilitating and potentially life-threatening muscle weakness. Approximately 85% of patients with gMG have IgG autoantibodies against skeletal muscle nicotinic acetylcholine receptor (AChR), muscle-specific tyrosine kinase (MuSK), and low-density lipoprotein receptor-related protein 4 (LRP4); of these, AChR antibodies are the most common.

Both conditions are associated with increased risk of infection—often due to immunological abnormalities and the immunosuppressive therapies used to treat them. Due to the immunocompromised status and baseline bulbar and respiratory muscle weakness, patients with gMG are at an increased risk of morbidity and mortality from respiratory diseases, including infection with severe acute respiratory syndrome coronavirus 2 (SARS-CoV-2), the cause of coronavirus disease 2019 (COVID-19). Long-term corticosteroid use and rituximab therapy are also predictive of severe COVID-19 infections in patients with gMG. Likewise in Pemphigus, data suggest immunosuppression with rituximab in this population may be a major risk factor for COVID-19.

Thus, there is a need for autoimmune disease treatments that reduce autoantibodies while still allowing the subject to mount an immune response to vaccines and to infectious agents.

Antagonism of FcRn results in a reduction in IgG levels and is being evaluated as a treatment approach for Pemphigus and gMG, among other conditions. Efgartigimod, currently approved in the U.S., EP, and JP for gMG treatment and in clinical trials for treatment of Pemphigus and other conditions, is a human IgG1 antibody Fc-fragment that has been engineered to have increased affinity to FcRn compared to endogenous IgG while retaining the characteristic pH dependence. However, concern has been raised about vaccination of subjects while receiving an FcRn antagonist due to their mechanism of action. Generally, current recommendations are to administer live attenuated vaccines at least 8 weeks prior to initiating FcRn antagonist treatment and to administer recombinant or inactive vaccines at least 2 to 4 weeks prior to initiating FcRn antagonist treatment. For those already receiving FcRn antagonist treatment, it is currently recommended to delay administering a vaccine until 2 months or more after receiving the last dose of FcRn antagonist to allow time for IgG restoration in these subjects.

### SUMMARY

The instant disclosure is broadly directed to methods of administering an FcRn antagonist and a vaccine to a subject less than 2 months apart from each other. It has been surprisingly shown herein that subjects are able to mount IgG responses to vaccines while receiving FcRn antagonist therapy, even when vaccination occurs less than 1 week before or after administration of an FcRn antagonist.

Thus, in one aspect, the instant disclosure provides a method of reducing serum IgG levels in a subject having an antibody-mediated disorder, comprising administering an effective amount of an FcRn antagonist to the subject within 4 weeks of administration of a vaccine to the subject.

In another aspect, the instant disclosure provides a method of vaccinating a subject having an antibody-mediated disorder, comprising administering a vaccine to the subject within 4 weeks of administration of an FcRn antagonist to the subject.

In some embodiments, the FcRn antagonist is administered to the subject ≤4 weeks after administration of the vaccine to the subject. In some embodiments, the FcRn antagonist is administered to the subject ≤2 weeks or ≤1 week after administration of the vaccine to the subject. In some embodiments, the FcRn antagonist is administered to the subject ≤24 hours after administration of the vaccine to the subject.

In some embodiments, an initial dose of the FcRn antagonist is administered to the subject after administration of the vaccine to the subject.

In another aspect, the instant disclosure provides a method of reducing serum IgG levels in a subject having an antibody-mediated disorder, comprising administering an effective amount of an FcRn antagonist to the subject ≤2 months before administration of a vaccine to the subject.

In another aspect, the instant disclosure provides a method of vaccinating a subject having an antibody-mediated disorder, comprising administering a vaccine to the subject ≤2 months after administration of an FcRn antagonist to the subject.

In some embodiments, the FcRn antagonist is administered to the subject ≤4 weeks, ≤2 weeks, or ≤1 week before administration of the vaccine to the subject. In some embodiments, the FcRn antagonist is administered to the subject ≤24 hours before administration of the vaccine to the subject. In some embodiments, the FcRn antagonist is administered to the subject on the same day as administration of the vaccine to the subject.

In some embodiments, the administration of the FcRn antagonist comprises administration of an initial dose of the FcRn antagonist and one or more subsequent doses of the FcRn antagonist. In some embodiments, a subsequent dose of the FcRn antagonist is administered to the subject ≤4 weeks after administration of the vaccine to the subject. In some embodiments, a subsequent dose of the FcRn antagonist is administered to the subject ≤2 weeks or ≤1 week after administration of the vaccine to the subject. In some embodiments, a subsequent dose of the FcRn antagonist is administered to the subject ≤24 hours after administration of the vaccine to the subject. In some embodiments, a subsequent dose of the FcRn antagonist is administered to the subject on the same day as administration of the vaccine to the subject.

In some embodiments, the FcRn antagonist is administered to the subject at a fixed dose of 20 mg to 20,000 mg or at a dose of 0.2 mg/kg to 200 mg/kg.

In some embodiments, the FcRn antagonist is administered intravenously at a dose of 2 mg/kg to 200 mg/kg once weekly or once every 2 weeks. In some embodiments, the FcRn antagonist is administered intravenously at a dose of 3 mg/kg to 60 mg/kg once weekly or once every 2 weeks. In some embodiments, the FcRn antagonist is administered intravenously at a dose of 10 mg/kg once weekly or once every 2 weeks. In some embodiments, the FcRn antagonist is administered intravenously at a dose of 25 mg/kg once weekly or once every 2 weeks.

In some embodiments, the FcRn antagonist is administered subcutaneously at a fixed dose of 200 mg to 20,000 mg once weekly, once every 2 weeks, once every 3 weeks, once every 4 weeks, once monthly, or once every 6 weeks. In some embodiments, the FcRn antagonist is administered subcutaneously at a fixed dose of 750 to 3000 mg once weekly, once every 2 weeks, once every 3 weeks, once every 4 weeks, once monthly, or once every 6 weeks. In some embodiments, the FcRn antagonist is administered subcutaneously at a fixed dose of 1000 mg or 2000 mg once weekly, once every 2 weeks, once every 3 weeks, once every 4 weeks, once monthly, or once every 6 weeks. In some embodiments, the FcRn antagonist is first administered subcutaneously at a fixed dose of 1000 mg twice on the same day. In some embodiments, the FcRn antagonist is administered subcutaneously at a fixed dose of 1000 mg once weekly.

In some embodiments, the FcRn antagonist is administered for 52 weeks or less. In some embodiments, the FcRn antagonist is administered for 26 weeks or less. In some embodiments, the FcRn antagonist is administered for 4 weeks or less.

In some embodiments, the FcRn antagonist is administered for at least 4 weeks. In some embodiments, the FcRn antagonist is administered for at least 26 weeks. In some embodiments, the FcRn antagonist is administered for at least 52 weeks.

In some embodiments, the FcRn antagonist is administered using a phased dosing schedule comprising a first treatment cycle and one or more subsequent treatment cycles, wherein the first treatment cycle and the one or more subsequent treatment cycles each independently comprise administration of 1-5 doses of the FcRn antagonist within 1 month. In some embodiments, the first treatment cycle comprises weekly administration of the FcRn antagonist for 4 weeks. In some embodiments, the one or more subsequent treatment cycles each comprise weekly administration of the FcRn antagonist for 4 weeks. In some embodiments, each of the one or more subsequent treatment cycles is administered to the subject based on clinical evaluation. In some embodiments, each of the one or more subsequent treatment cycles is administered ≥50 days from the start of the previous treatment cycle.

In some embodiments, the FcRn antagonist comprises two, three, or four FcRn binding regions. In some embodiments, the FcRn antagonist comprises or consists of a variant Fc region or FcRn binding fragment thereof. In some embodiments, the variant Fc region or FcRn binding fragment thereof binds to FcRn with a higher affinity at pH 6.0 as compared to a corresponding wild-type Fc region. In some embodiments, the variant Fc region or FcRn binding fragment thereof binds to FcRn with a higher affinity at pH 7.4 as compared to a corresponding wild-type Fc region.

In some embodiments, the variant Fc region comprises or consists of a first Fc domain and a second Fc domain which form a homodimer or heterodimer. In some embodiments, the first Fc domain and/or the second Fc domain comprise amino acids Y, T, E, K, and F at EU positions 252, 254, 256, 433, and 434, respectively. In some embodiments, the first Fc domain and/or the second Fc domain comprise amino acids Y, T, E, K, F, and Y at EU positions 252, 254, 256, 433, 434, and 436, respectively.

In some embodiments, the first Fc domain and/or the second Fc domain comprise an amino acid sequence independently selected from the group consisting of SEQ ID NO: 2, SEQ ID NO: 3, SEQ ID NO: 20, and SEQ ID NO: 21. In some embodiments, the first Fc domain and the second Fc domain comprise an amino acid sequence independently selected from the group consisting of SEQ ID NO: 2, SEQ ID NO: 3, SEQ ID NO: 20, and SEQ ID NO: 21.

In some embodiments, the FcRn antagonist is efgartigimod.

In some embodiments, the FcRn antagonist is an anti-FcRn antibody.

In some embodiments, the FcRn antagonist is administered to the subject as part of a composition comprising a population of FcRn antagonist molecules, wherein at least a portion of the FcRn antagonist molecules in the population consist of a variant Fc region comprising a dimer of a first Fc domain and a second Fc domain, wherein the amino acid sequence of the first and second Fc domain consists of SEQ ID NO: 1, provided that the population is not a homogeneous population of homodimeric FcRn antagonist molecules in which the amino acid sequence of both the first and the second Fc domain consists of SEQ ID NO: 2, 3, 20, or 21, wherein the population comprises:
(a) a first subpopulation of FcRn antagonist molecules, wherein the amino acid sequences of both the first and the second Fc domains of the FcRn antagonist molecules in the first subpopulation consist of SEQ ID NO: 3; and
(b) at least one of:
   (i) a second subpopulation of FcRn antagonist molecules, wherein the amino acid sequences of the first and the second Fc domains of the FcRn antagonist molecules in the second subpopulation consist of SEQ ID NO: 3 and SEQ ID NO: 12, respectively;
   (ii) a third subpopulation of FcRn antagonist molecules, wherein the amino acid sequences of the first and the second Fc domains of the FcRn antagonist molecules in the third subpopulation consist of SEQ ID NO: 3 and SEQ ID NO: 9, respectively;
   (iii) a fourth subpopulation of FcRn antagonist molecules, wherein the amino acid sequences of both the first and the second Fc domains of the FcRn antagonist molecules in the fourth subpopulation consist of SEQ ID NO: 3, and wherein two asparagine residues in each FcRn antagonist molecule in the fourth subpopulation are deaminated;
   (iv) a fifth subpopulation of FcRn antagonist molecules, wherein the amino acid sequences of the first and the second Fc domains of the FcRn antagonist molecules in the fifth subpopulation consist of SEQ ID NO: 3, and wherein one asparagine residue in each FcRn antagonist molecule in the fifth subpopulation is deaminated;
   (v) a sixth subpopulation of FcRn antagonist molecules, wherein the amino acid sequences of the first and the second Fc domains of the FcRn antagonist molecules in the sixth subpopulation consist of SEQ ID NO: 2 and SEQ ID NO: 3, respectively;
   (vi) a seventh subpopulation of FcRn antagonist molecules, wherein the amino acid sequences of the first and the second Fc domains of the FcRn antagonist molecules in the seventh subpopulation consist of SEQ ID NO: 3, and wherein one methionine residue or one tryptophan residue in each FcRn antagonist molecule in the seventh subpopulation is oxidized;
   (vii) an eighth subpopulation of FcRn antagonist molecules, wherein the amino acid sequences of both the first and the second Fc domains of the FcRn antagonist molecules in the eighth subpopulation consist of SEQ ID NO: 2;
   (viii) a ninth subpopulation of FcRn antagonist molecules, wherein the amino acid sequences of the first and the second Fc domains of the FcRn antagonist molecules in the ninth subpopulation consist of SEQ ID NO: 3 and SEQ ID NO: 6, respectively;
   (ix) a tenth subpopulation of FcRn antagonist molecules, wherein the amino acid sequences of the first and the second Fc domains of the FcRn antagonist molecules in the tenth subpopulation consist of SEQ ID NO: 2 and SEQ ID NO: 3, respectively, and wherein one methionine residue or one tryptophan residue in each FcRn antagonist molecule in the tenth subpopulation is oxidized; and
   (x) an eleventh subpopulation of FcRn antagonist molecules, wherein the amino acid sequences of both the first and the second Fc domains of the FcRn antagonist molecules in the eleventh subpopulation consist of SEQ ID NO: 3, and wherein two amino acid residues, independently selected from a methionine residue and a tryptophan, in each FcRn antagonist molecule in the eleventh subpopulation are oxidized.

In some embodiments, the vaccine is administered in a single dose. In some embodiments, the vaccine is administered in two or more doses. In some embodiments, the vaccine is administered in two doses. In some embodiments, the doses are administered more than 1 week apart. In some embodiments, the doses are administered more than 2 weeks apart. In some embodiments, the doses are administered less than 3 months apart. In some embodiments, the doses are administered less than 2 months or less than 1 month apart.

In some embodiments, the vaccine is selected from a recombinant vaccine, a toxoid vaccine, an inactivated vaccine, and a live attenuated vaccine. In some embodiments, the vaccine is a recombinant vaccine, a toxoid vaccine, or an inactivated vaccine. In some embodiments, the recombinant vaccine is selected from an mRNA vaccine, a viral vector vaccine, and a subunit vaccine. In some embodiments, the subunit vaccine is selected from a polysaccharide vaccine, a conjugate vaccine, and a protein-based vaccine.

In some embodiments, the vaccine is a T-cell dependent vaccine.

In some embodiments, the vaccine is a COVID-19 vaccine, an influenza vaccine, or a pneumococcal vaccine.

In some embodiments, the COVID-19 vaccine is selected from tozinameran, elasomeran, NVX-CoV2373, Ad26.COV2.S, AZD1222, CoronaVac, BBV152, AD5-nCOV, MVC-COV1901, BBIBP-CorV, and Gam-COVID-Vac.

In some embodiments, the influenza vaccine is selected from Audenz, Fluad Quadrivalent, Fluad, Afluria Quadrivalent, Afluria Quadrivalent Southern Hemisphere, Flucelvax Quadrivalent, Flulaval Quadrivalent, Afluria, Afluria Southern Hemisphere, FluLaval, FluMist, Fluarix, Fluvirin, Agriflu, Fluzone, Fluzone High-Dose, Fluzone Intradermal, Flucelvax, Flublok, Flublok Quadrivalent, FluMist Quadrivalent, Fluarix Quadrivalent, and Fluzone Quadrivalent.

In some embodiments, the pneumococcal vaccine is selected from Pneumovax 23, Prevnar 13, VAXNEUVANCE™, and Prevnar 20.

In some embodiments, administration of the vaccine elicits a protective immune response.

In some embodiments, the vaccine comprises one or more pathogenic antigens and IgG antibody titers against one or more of the pathogenic antigens are maintained at or above a protective threshold following administration of the FcRn antagonist to the subject.

In some embodiments, the vaccine is a pneumococcal conjugate vaccine, and the protective threshold is an IgG antibody concentration of ≥350 ng/mL. In some embodiments, the pneumococcal conjugate vaccine is a 13-valent pneumococcal conjugate vaccine (PCV13).

In some embodiments, the vaccine is an influenza vaccine, and the protective threshold is a neutralizing titer of ≥1:40, optionally measured using a hemagglutination inhibition assay.

In some embodiments, the vaccine is a COVID-19 vaccine, and the protective threshold is an anti-SARS-CoV-2 Spike protein receptor-binding domain (S-RBD) IgG level of ≥620.2 AU/mL. In some embodiments, the vaccine is a COVID-19 vaccine, and the protective threshold is an anti-S-RBD IgG level of ≥82.82 AU/mL.

In some embodiments, the vaccine comprises one or more pathogenic antigens, and IgG antibody titers against one or more of the pathogenic antigens are maintained at or above a baseline value following administration of the FcRn antagonist to the subject. In some embodiments, the baseline value is an IgG antibody titer against the one or more pathogenic antigens in the subject before administration of the FcRn antagonist to the subject.

In some embodiments, the subject has an antigen-specific IgG response following subsequent antigen challenge. In some embodiments, the subject shows at least a 4-fold increase in antigen-specific IgG following a second administration of the vaccine. In some embodiments, the subject shows at least a 20-fold increase in antigen-specific IgG following a second administration of the vaccine. In some embodiments, the antigen-specific IgG is an anti-S-RBD IgG.

In some embodiments, the vaccine comprises one or more SARS-CoV-2 antigens selected from the group consisting of spike protein S2, nucleocapsid, spike protein S1, full-length spike protein, and spike protein receptor-binding domain.

In some embodiments, the vaccine is not a seasonal vaccine.

In some embodiments, the antibody-mediated disorder is an IgG-mediated disorder. In some embodiments, the antibody-mediated disorder is an autoimmune disease.

In some embodiments, the autoimmune disease is selected from the group consisting of: allogenic islet graft rejection, alopecia areata, ankylosing spondylitis, antiphospholipid syndrome, autoimmune Addison's disease, Alzheimer's disease, antineutrophil cytoplasmic auto-antibodies (ANCA), autoimmune diseases of the adrenal gland, autoimmune hemolytic anemia, autoimmune hepatitis, autoimmune myocarditis, autoimmune neutropenia, autoimmune oophoritis and orchitis, immune thrombocytopenia (ITP or idiopathic thrombocytopenic purpura, idiopathic thrombocytopenia purpura, immune mediated thrombocytopenia, or primary immune thrombocytopenia), autoimmune urticaria, Behcet's disease, bullous pemphigoid (BP), cardiomyopathy, Castleman disease, celiac sprue-dermatitis, chronic fatigue immune dysfunction syndrome, chronic inflammatory demyelinating polyneuropathy (CIDP), Churg-Strauss syndrome, cicatricial pemphigoid, CREST syndrome, cold agglutinin disease, Crohn's disease, dilated cardiomyopathy, discoid lupus, epidermolysis bullosa acquisita, essential mixed cryoglobulinemia, factor VIII deficiency, fibromyalgia-fibromyositis, glomerulonephritis, Goodpasture syndrome, Graves' disease, Guillain-Barre syndrome, graft-versus-host disease (GVHD), Hashimoto's thyroiditis, hemophilia A, idiopathic inflammatory myopathies (IIMs), idiopathic membranous neuropathy, idiopathic pulmonary fibrosis, IgA neuropathy, IgM polyneuropathies, immune-mediated necrotizing myopathy (IMNM), juvenile arthritis, Kawasaki disease, lichen planus, lichen sclerosus, lupus erythematosus, lupus nephritis, Ménière's disease, mixed connective tissue disease, mucous membrane pemphigoid, multiple sclerosis, Type 1 diabetes mellitus, multifocal motor neuropathy (MMN), myasthenia gravis (MG), generalized myasthenia gravis (gMG), myositis, paraneoplastic bullous pemphigoid, pemphigoid gestationis, Pemphigus vulgaris (PV), Pemphigus foliaceus (PF), pernicious anemia, polyarteritis nodosa, polychondritis, polyglandular syndromes, polymyalgia rheumatica, polymyositis, dermatomyositis (DM), necrotizing autoimmune myopathy (NAM), AntiSynthetase Syndrome (ASyS), primary agammaglobulinemia, primary biliary cirrhosis, psoriasis, psoriatic arthritis, relapsing polychondritis, Raynaud's phenomenon, Reiter's syndrome, rheumatoid arthritis, sarcoidosis, scleroderma, Sjögren's syndrome, solid organ transplant rejection, stiff-person syndrome, systemic lupus erythematosus, Takayasu's arteritis, toxic epidermal necrolysis (TEN), Stevens-Johnson syndrome (SJS), temporal arteritis/giant cell arteritis, thrombotic thrombocytopenia purpura, ulcerative colitis, uveitis, dermatitis herpetiformis vasculitis, anti-neutrophil cytoplasmic antibody-associated vasculitides, vitiligo, and Wegener's granulomatosis.

In some embodiments, the autoimmune disease is gMG, PV, or PF.

In some embodiments, the autoimmune disease is not gMG, PV, or PF.

In some embodiments, the autoimmune disease is selected from the group consisting of: allogenic islet graft rejection, alopecia areata, ankylosing spondylitis, antiphospholipid syndrome, autoimmune Addison's disease, Alzheimer's disease, ANCA, autoimmune diseases of the adrenal gland, autoimmune hemolytic anemia, autoimmune hepatitis, autoimmune myocarditis, autoimmune neutropenia, autoimmune oophoritis and orchitis, immune thrombocytopenia (ITP or idiopathic thrombocytopenic purpura, idiopathic thrombocytopenia purpura, immune mediated thrombocytopenia, or primary immune thrombocytopenia), autoimmune urticaria, Behcet's disease, BP, cardiomyopathy, Castleman disease, celiac sprue-dermatitis, chronic fatigue immune dysfunction syndrome, CIDP, Churg-Strauss syndrome, cicatricial pemphigoid, CREST syndrome, cold agglutinin disease, Crohn's disease, dilated cardiomyopathy, discoid lupus, epidermolysis bullosa acquisita, essential mixed cryoglobulinemia, factor VIII deficiency, fibromyalgia-fibromyositis, glomerulonephritis, Goodpasture syndrome, Graves' disease, Guillain-Barre syndrome, GVHD, Hashimoto's thyroiditis, hemophilia A, IIMs, idiopathic membranous neuropathy, idiopathic pulmonary fibrosis, IgA neuropathy, IgM polyneuropathies, IMNM, juvenile arthritis, Kawasaki disease, lichen planus, lichen sclerosus, lupus erythematosus, lupus nephritis, Ménière's disease, mixed connective tissue disease, mucous membrane pemphigoid, multiple sclerosis, Type 1 diabetes mellitus, MMN, myositis, paraneoplastic bullous pemphigoid, pemphigoid gestationis, pernicious anemia, polyarteritis nodosa, polychondritis, polyglandular syndromes, polymyalgia rheumatica, polymyositis, DM, NAM, ASyS, primary agammaglobulinemia, primary biliary cirrhosis, psoriasis, psoriatic arthritis, relapsing polychondritis, Raynaud's phenomenon, Reiter's syndrome, rheumatoid arthritis, sarcoidosis, scleroderma, Sjögren's syndrome, solid organ transplant rejection, stiff-person syndrome, systemic lupus erythematosus, Takayasu's arteritis, TEN, SJS, temporal arteritis/giant cell arteritis, thrombotic thrombocytopenia purpura, ulcerative colitis, uveitis, dermatitis herpetiformis vasculitis, anti-neutrophil cytoplasmic anti-body-associated vasculitides, vitiligo, and Wegener's granulomatosis.

The instant disclosure also provides an FcRn antagonist for use in the treatment of an antibody-mediated disorder, wherein the treatment is performed according to the methods described above and herein.

The instant disclosure also provides an FcRn antagonist for use in the manufacture of a medicament for the treatment of an antibody-mediated disorder, wherein the treatment is performed according to the methods described above and herein.

The instant disclosure also provides use of an FcRn antagonist in the treatment of an antibody-mediated disorder, wherein the treatment is performed according to the methods described above and herein.

The instant disclosure also provides use of an FcRn antagonist in the manufacture of a medicament for the treatment of an antibody-mediated disorder, wherein the treatment is performed according to the methods described above and herein.

The instant disclosure also provides a vaccine for use in the vaccination of a subject having an antibody-mediated disorder, wherein the vaccination is performed according to the methods described above and herein.

The instant disclosure also provides use of a vaccine in the vaccination of a subject having an antibody-mediated disorder, wherein the vaccination is performed according to the methods described above and herein.

### BRIEF DESCRIPTION OF THE DRAWINGS

FIG. 1 is a schematic illustration of the design of the TDAR (T-cell Dependent Antibody Response) study in cynomolgus monkeys, aimed to assess the influence of efgartigimod (ARGX-113) on keyhole limpet hemocyanin (KLH) immunization.

FIG. 2 is graph showing exemplary pharmacokinetics of efgartigimod (ARGX-113) in efgartigimod-treated cynomolgus monkeys.

FIGS. 3A-3D present exemplary anti-KLH IgG responses in efgartigimod-treated or untreated cynomolgus monkeys subjected to KLH immunization. FIG. 3A is a graph showing anti-KLH IgG titer in untreated cynomolgus monkeys subjected to KLH immunization. FIG. 3B is a graph showing anti-KLH titer in efgartigimod-treated cynomolgus monkeys subjected to KLH immunization. FIG. 3C is a graph combining the data shown in FIGS. 3A and 3B. FIG. 3D is a graph showing average anti-KLH IgG titers in male and female cynomolgus monkeys subjected to KLH immunization, either efgartigimod-treated or untreated. In these figures, the efgartigimod dosing phase and recovery phase is indicated and separated by a dotted line. Day 1 of the recovery phase immediately followed the last day (Day 77) of the dosing phase (see FIG. 1). KLH immunization occurred on Days 28 & 56 of the dosing phase and on Day 29 of the recovery phase (circled).

FIGS. 4A-4D present exemplary anti-KLH IgM responses in efgartigimod-treated or untreated cynomolgus monkeys subjected to KLH immunization. FIG. 4A is a graph showing anti-KLH IgM titer in untreated cynomolgus monkeys subjected to KLH immunization. FIG. 4B is a graph showing anti-KLH IgM titer in efgartigimod-treated cynomolgus monkeys subjected to KLH immunization. FIG. 4C is a graph combining the data shown in FIGS. 4A and 4B. FIG. 4D is a graph showing average anti-KLH IgM titers in male and female cynomolgus monkeys subjected to KLH immunization, either efgartigimod-treated or untreated. In these figures, the efgartigimod dosing phase and recovery phase is indicated and separated by a dotted line. Day 1 of the recovery phase immediately followed the last day (Day 77) of the dosing phase (see FIG. 1). KLH immunization occurred on Days 28 & 56 of the dosing phase and on Day 29 of the recovery phase (circled).

FIGS. 5A-5B present exemplary total IgG levels in efgartigimod-treated or untreated cynomolgus monkeys subjected to KLH immunization. FIG. 5A is a graph showing total IgG as percentage from predose (% predose) over the course of the study. FIG. 5B is a graph showing total IgG in g/L over the course of the study. In these figures, KLH immunization occurred on Days 28 & 56 of the dosing phase and on Day 29 of the recovery phase (i.e., study day 106), and is indicated by arrows on the x-axis. The dosing and recovery phases are separated by a dotted line.

FIGS. 6A-6D present exemplary cellular immune responses in efgartigimod-treated or untreated cynomolgus monkeys subjected to KLH immunization. In these figures, cellular responses are indicated by the measurement of spot forming units (SFU) per million of peripheral blood mononuclear cells (PBMCs) (SFU/million PBMCs, y-axis). FIG. 6A is a graph showing cellular immune responses after KLH recall challenge, in efgartigimod-treated or untreated cynomolgus monkeys, subjected to KLH immunization. FIG. 6B is a graph showing cellular immune response in positive control experiments with phytohemagglutinin (PHA), in efgartigimod-treated or untreated cynomolgus monkeys subjected to KLH immunization. FIG. 6C is a graph showing cellular immune response in negative control experiments in efgartigimod-treated or untreated cynomolgus monkeys subjected to KLH immunization. FIG. 6D is a graph showing cellular immune responses after KLH recall challenge in comparison to negative control experiments in efgartigimod-treated or untreated cynomolgus monkeys subjected to KLH immunization. In these figures, the efgartigimod dosing phase and recovery phase is indicated and separated by a dotted line.

FIG. 7 is a schematic illustration of Pemphigus and generalized myasthenia gravis (gMG) trials, trial dosing, and post-hoc analysis conducted.

FIGS. 8A-8B are graphs presenting exemplary data showing the effects of efgartigimod on different and total IgG levels in treated subjects. FIG. 8A shows mean percent change from baseline for anti-varicella zoster virus (VZV), anti-tetanus toxin (TT), anti-pneumococcal capsular polysaccharide (PCP), anti-desmoglein 1 (Dsg1), anti-desmoglein 3 (Dsg3), and total IgG antibodies in 15 patients treated for up to 238 days (34 weeks) with efgartigimod. For anti-Dsg 1/3 antibodies, data is plotted for patients who presented with positive anti-Dsg1/3 autoantibody levels ≥20 U/mL at baseline (n=13 for anti-Dsg1, n=8 for anti-Dsg3), excluding one patient who presented with positive anti-Dsg3 autoantibody levels only on day 235 when relapse occurred. Error bars represent standard error of the mean (SEM). FIG. 8B shows percent change from baseline for anti-VZV and total IgG in a patient with PV exhibiting a transient increase in anti-VZV IgG titers. In both figures, the efgartigimod treatment period is indicated by background shading.

FIGS. 9A-9G are graphs presenting exemplary data showing the levels of SARS-CoV-2 S-protein receptor-binding domain (S-RBD) IgG before the first and after the second dose (Day 1) of BNT162b2 (FIGS. 9A-9F) or mRNA-1273 (FIG. 9G) in individual patients with generalized myasthenia gravis (gMG) who participated in ADAPT+. Shaded areas represent efgartigimod treatment cycles in FIGS. 9A-9G. In each figure, the dashed line represents the lower limit of quantitation (LLOQ). The lowest total IgG levels are expected 1 week after the last efgartigimod infusion of each cycle, which is indicated by arrows.

FIGS. 10A-10D are graphs presenting exemplary data showing individual hemagglutination inhibition (HI) titers from 7 patients with gMG vaccinated against influenza during the double-blind treatment period or open-label extension of ADAPT. FIG. 10A shows HI titers for patients who received only placebo. FIG. 10B shows HI titers for patients treated with efgartigimod and vaccinated between treatment cycles. FIG. 10C shows HI titers for patients treated with efgartigimod and vaccinated within a treatment cycle. FIG. 10D shows HI titers for an efgartigimod-treated patient who was vaccinated but infected with influenza. In each figure, the protection threshold (PT) and upper limit of quantification (ULQ) are indicated by dashed lines.

FIGS. 11A-11B are graphs presenting exemplary data showing anti-pneumococcal capsular polysaccharide (PCP) titers against different PCP serotypes in two different efgartigimod-treated patients after vaccination with a pneumococcal conjugate vaccine. FIG. 11A shows anti-PCP titers against 13 PCP serotypes in an efgartigimod-treated patient after vaccination with 13-valent pneumococcal conjugate vaccine (PCV13). FIG. 11B shows anti-PCP titers against 12 serotypes in an efgartigimod-treated patient after vaccination with 23-valent pneumococcal polysaccharide vaccine (PPSV23). In both figures, shaded areas represent efgartigimod treatment periods, and the PT (350 ng/mL) is shown as a dotted line.

FIG. 12 presents graphs showing individual HI titers from 4 patients with gMG who were vaccinated against influenza during the double-blind trial ADAPT.

FIG. 13 is a graph showing the mean percent change in total IgG levels and levels of antibodies (Ab) against the skeletal muscle nicotinic acetylcholine receptor (AChR) in AChR-Ab+ patients, over time (10 weeks), in cycle 1 of the double-blind trial ADAPT.

FIGS. 14A-14C present graphs showing exemplary levels of SARS-CoV-2 S-RBD IgG in patients from the ADAPT+ trial that received COVID-19 vaccination. Shaded areas represent efgartigimod therapy treatment cycles. In each graph, the dashed line represents the LLOQ. Any additional treatment a given patient may have received, such as mycophenolate mofetil (MMF), azathioprine, or prednisone is indicated next to its respective graph. In these figures, “qd” means “quaque die” (once a day) and “bid” means “bis in die” (twice a day).

### DETAILED DESCRIPTION

The instant disclosure is broadly directed to methods of administering an FcRn antagonist and a vaccine to a subject less than two months apart from each other based on the surprising discovery that subjects are able to mount IgG responses to vaccines while receiving FcRn antagonist therapy, even when vaccination occurs less than 1 week before or after administration of an FcRn antagonist. Thus, the instant disclosure provides methods for vaccination of subjects treated with an FcRn antagonist. The present disclosure also provides methods of administering an FcRn antagonist to a subject that has recently received a vaccine or will soon receive a vaccine. In some embodiments, the FcRn antagonist is efgartigimod.

### Definitions

As used herein, the term “FcRn” refers to a neonatal Fc receptor. Exemplary FcRn molecules include human FcRn encoded by the FCGRT gene as set forth in RefSeq NM 004107. The amino acid sequence of the corresponding protein is set forth in RefSeq NP_004098.

As used herein, the term “FcRn antagonist” refers to any agent that binds specifically to FcRn and inhibits the binding of immunoglobulin to FcRn (e.g., human FcRn). In an embodiment, the FcRn antagonist is an Fc region (e.g., a variant Fc region disclosed herein) that specifically binds to FcRn through the Fc region and inhibits the binding of immunoglobulin to FcRn. In an embodiment, the FcRn antagonist is not a full-length IgG antibody. In an embodiment, the FcRn antagonist comprises an antigen binding site that binds a target antigen and a variant Fc region. In an embodiment, the FcRn antagonist is an Fc fragment comprising or consisting of an Fc region and lacking an antigen binding site. In an embodiment, the term “FcRn antagonist” refers to an antibody or antigen-binding fragment thereof that specifically binds to FcRn via its antigen binding domain or via its Fc region and inhibits the binding of the Fc region of immunoglobulin (e.g., IgG autoantibodies) to FcRn.

As used herein, the terms “antibody” and “antibodies” include full-length antibodies, antigen-binding fragments of full-length antibodies, and molecules comprising antibody CDRs, VH regions, or VL regions. Examples of antibodies include monoclonal antibodies, recombinantly produced antibodies, monospecific antibodies, multi-specific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies (sdAb), monovalent antibodies, single chain antibodies or single-chain Fvs (scFv), camelid antibodies, affibody molecules, humanized antibodies, VHH fragments, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. Antibodies can be of any type (e.g., IgG, IgE, IgM, IgD, IgA, or IgY), any class (e.g., IgG1, IgG2, IgG3, IgG4, IgAQ1, or IgA2), or any subclass (e.g., IgG2a or IgG2b) of immunoglobulin molecule.

As used herein, the term “Fc domain” refers to the portion of a single immunoglobulin heavy chain comprising both the CH2 and CH3 domains of the antibody. In some embodiments, the Fc domain comprises at least a portion of a hinge (e.g., upper, middle, and/or lower hinge region) region, a CH2 domain, and a CH3 domain. In some embodiments, the Fc domain does not include the hinge region.

As used herein, the term “hinge region” refers to the portion of a heavy chain molecule that joins the CH1 domain to the CH2 domain. In some embodiments, the hinge region is at most 70 amino acid residues in length. In some embodiments, this hinge region comprises approximately 11-17 amino acid residues and is flexible, thus allowing the two N-terminal antigen binding regions to move independently. In some embodiments, the hinge region is 12 amino acid residues in length. In some embodiments, the hinge region is 15 amino acid residues in length. In some embodiments, the hinge region is 62 amino acid residues in length. Hinge regions can be subdivided into three distinct domains: upper, middle, and lower hinge domains. The FcRn antagonists of the instant disclosure can include all or any portion of a hinge region. In some embodiments, the hinge region is from an IgG1 antibody. In some embodiments, the hinge region comprises the amino acid sequence of EPKSCDKTHTCPPCP (SEQ ID NO: 31).

As used herein, the term “Fc region” refers to the portion of an immunoglobulin formed by the Fc domains of its two heavy chains. The Fc region can be a wild-type Fc region (native Fc region) or a variant Fc region. A native Fc region is homodimeric. The Fc region can be derived from any native immunoglobulin. In some embodiments, the Fc region is formed from an IgA, IgD, IgE, or IgG heavy chain constant region. In some embodiments, the Fc region is formed from an IgG heavy chain constant region. In some embodiments, the IgG heavy chain constant region is an IgG1, IgG2, IgG3, or IgG4 heavy chain constant region. In some embodiments, the Fc region is formed from an IgG1 heavy chain constant region. In some embodiments, the IgG1 heavy chain constant region comprises a G1m1(a), G1m2(x), G1m3(f), or G1m17(z) allotype. See, e.g., Jefferis and Lefranc (2009) mAbs 1(4):332-338, and de Taeye et al., (2020) Front Immunol. 11:740, incorporated herein by reference in their entirety.

As used herein, the term “variant Fc region” refers to an Fc region with one or more alteration(s) relative to a native Fc region. Alterations can include amino acid substitutions, additions and/or deletions, linkage of additional moieties, and/or alteration of the native glycans. The term encompasses heterodimeric Fc regions where each of the constituent Fc domains is different. The term also encompasses single chain Fc regions where the constituent Fc domains are linked together by a linker moiety.

As used herein, the term “FcRn binding fragment” refers to a portion of an Fc region that is sufficient to confer FcRn binding.

As used herein, the term “EU position” refers to the amino acid position in the EU numbering convention for the Fc region described in Edelman, G M et al., Proc. Natl. Acad. USA, 63, 78-85 (1969) and Rabat et al., in “Sequences of Proteins of Immunological Interest,” U.S. Dept. Health and Human Services, 5th edition, 1991.

As used herein, the term “baseline” refers to a measurement (e.g., IgG levels) in a patient (e.g., in a patient's blood or urine) prior to the first administration (e.g., intravenous or subcutaneous administration) of a treatment (e.g., an FcRn antagonist).

As used herein, the term “autoantibody-mediated disease” refers to any disease or disorder in which the underlying pathology is caused, at least in part, by pathogenic IgG autoantibodies.

As used herein, the terms “treat,” “treating,” and “treatment” refer to therapeutic or preventative measures described herein. The methods of “treatment” employ administration of a polypeptide to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the terms “dose” or “dosing” refer to an amount of an agent administered to a subject in a single administration.

As used herein, the terms “fixed dose” or “flat dose” both refer to a dose that does not vary based upon a characteristic (e.g., body mass, e.g., within a set range; sex; age, e.g., within a set range; etc.) of the subject.

As used herein, the term “subject” or “patient” or “participant” includes any human or non-human animal. In an embodiment, the subject or patient or participant is a human or non-human mammal. In an embodiment, the subject or patient or participant is a human.

As used herein, the term “about” or “approximately” when referring to a measurable value, such as a dosage, encompasses variations of ±5% of a given value or range, as are appropriate to perform the methods disclosed herein.

### FcRn Antagonists

FcRn antagonists that are useful in the methods and uses provided herein include any molecule that binds to and inhibits FcRn, including, but not limited to, any anti-FcRn antibody, any anti-FcRn binding region, or any Fc domain or Fc region.

In some embodiments, the FcRn antagonists disclosed herein comprise two, three, or four FcRn binding regions, such as an Fc region.

Any Fc region can be altered to produce a variant Fc region for use in the methods disclosed herein. In general, an Fc region, or FcRn binding fragment thereof, is from a human immunoglobulin. It is understood, however, that the Fc region may be derived from an immunoglobulin of any other mammalian species, including for example, a camelid species, a rodent (e.g., a mouse, rat, rabbit, guinea pig), or non-human primate (e.g., chimpanzee, macaque) species. Moreover, the Fc region or portion thereof may be derived from any immunoglobulin class, including IgM, IgG, IgD, IgA, and IgE, and any immunoglobulin isotype, including IgG1, IgG2, IgG3, and IgG4. In an embodiment, the Fc region is an IgG Fc region (e.g., a human IgG region). In an embodiment, the Fc region is an IgG1 Fc region (e.g., a human IgG1 region). In an embodiment, the Fc region is a chimeric Fc region comprising portions of several different Fc regions. Suitable examples of chimeric Fc regions are set forth in US 2011/0243966A1, which is incorporated herein by reference in its entirety. A variety of Fc region gene sequences (e.g., human constant region gene sequences) are available in the form of publicly accessible deposits.

An Fc region can be further truncated or internally deleted to produce a minimal FcRn binding fragment thereof. The ability of an Fc-region fragment to bind to FcRn can be determined using any art recognized binding assay (e.g., ELISA).

To enhance the manufacturability of the FcRn antagonists disclosed herein, it is preferable that the constituent Fc regions do not comprise any non-disulfide bonded cysteine residues. Accordingly, in an embodiment, the Fc regions do not comprise a free cysteine residue.

Any Fc variant, or FcRn binding fragment thereof, that binds specifically to FcRn with increased affinity and reduced pH dependence relative to the native (i.e., wild-type) Fc region can be used in the methods disclosed herein. In an embodiment, the variant Fc region comprises amino acid alterations, substitutions, insertions, and/or deletions that confer the desired characteristics. In some embodiments, the FcRn antagonist comprises a variant Fc region, or FcRn binding fragment thereof, which binds to FcRn with a higher affinity at pH 5.5 as compared to a corresponding wild-type Fc region. In some embodiments, the FcRn antagonist comprises or consists of a variant Fc region, or FcRn binding fragment thereof, which binds to FcRn with a higher affinity at pH 6.0 and/or at pH 7.4 as compared to a corresponding wild-type Fc region. In some embodiments, the FcRn antagonist comprises a variant Fc region, or FcRn binding fragment thereof, which binds to FcRn with a higher affinity at both acidic and neutral pH.

In some embodiments, the variant Fc region is derived from the Fc region of any native immunoglobulin. In some embodiments, the native immunoglobulin is a human immunoglobulin. In some embodiments, the immunoglobulin is IgA, IgD, IgE, or IgG. In some embodiments, the immunoglobulin is IgG. In some embodiments, the immunoglobulin is human IgA, human IgD, human IgE, or human IgG. In some embodiments, the immunoglobulin is human IgG. In some embodiments, the IgG is IgG1, IgG2, IgG3, or IgG4. In some embodiments, the human IgG is human IgG1, human IgG2, human IgG3, or human IgG4. In some embodiments, the variant Fc region varies from the human IgG1 Fc region. In some embodiments, the human IgG1 Fc region comprises a G1m1(a), G1m2(x), G1m3(f), or G1m17(z) allotype.

In an embodiment, the variant Fc region, or FcRn binding fragment thereof consists of two Fc domains.

In an embodiment, the variant Fc region comprises or consists of a first Fc domain and a second Fc domain which form a homodimer or heterodimer. In an embodiment, the first Fc domain and/or the second Fc domain comprise amino acids Y, T, E, K, and F at EU positions 252, 254, 256, 433, and 434, respectively. In an embodiment, the first Fc domain and/or the second Fc domain comprise amino acids Y, T, E, K, F, and Y at EU positions 252, 254, 256, 433, 434, and 436, respectively.

In some embodiments, the FcRn antagonists disclosed herein comprise or consist of at least one Fc domain, wherein the amino acid sequence of the at least one Fc domain comprises or consists of the amino acid sequence of SEQ ID NO:1, provided below.

TABLE 1

| | SEQ |
|---|---|
| Amino Acid Sequence | ID NO: |
| X₁X₂X₃X₄X₅X₆PPCPAPELLGGPSVFLFPPKPKDTLYITREPEVTCVVVDVSHE | 1 |
| DPEVKFNWYVDGVEVHNAKTKPREEQYNSTYRVVSVLTVLHQDWLNGKE | |
| YKCKVSNKALPAPIEKTISKAKGQPREPQVYTLPPSRDELTKNQVSLTCLVK | |
| GFYPSDIAVEWESNGQPENNYKTTPPVLDSDGSFFLYSKLTVDKSRWQQGN | |
| VFSCSVMHEALKFHYTQKSLSLSPX₇X₈ | |
| wherein: X₁ is D or absent; X₂ is K or absent; X₃ is T or | |
| absent; X₄ is H or absent; X₅ is T or absent; X₆ is C or | |
| absent; X₇ is G or absent; X₈ is K or absent. | |

In some embodiments, the FcRn antagonists disclosed herein comprise or consist of a variant Fc region comprising or consisting of a dimer of a first Fc domain and a second Fc domain, wherein the amino acid sequence of the first and second Fc domain comprises or consists of the amino acid sequence of SEQ ID NO: 1.

In some embodiments, the FcRn antagonists disclosed herein comprise or consist of an amino acid sequence at least 70%, 75%, 80%, 85%, 90%, 95%, 96%, 97%, 98%, 99%, or 100% identical to the amino acid sequence of a variant Fc region comprising or consisting of a dimer of a first Fc domain and a second Fc domain, wherein the amino acid sequence of the first and second Fc domain comprises or consists of an amino acid sequence independently selected from the group consisting of SEQ ID NOs: 2-22. In some embodiments, the dimer is a heterodimer or a homodimer.

TABLE 2

| | SEQ |
|---|---|
| Amino Acid Sequence | ID NO: |
| DKTHTCPPCPAPELLGGPSVFLFPPKPKDTLYITREPEVTCVVVDVSHEDPE | 2 |
| VKFNWYVDGVEVHNAKTKPREEQYNSTYRVVSVLTVLHQDWLNGKEYKC | |
| KVSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSPGK | |
| DKTHTCPPCPAPELLGGPSVFLFPPKPKDTLYITREPEVTCVVVDVSHEDPE | 3 |
| VKFNWYVDGVEVHNAKTKPREEQYNSTYRVVSVLTVLHQDWLNGKEYKC | |
| KVSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSPG | |
| DKTHTCPPCPAPELLGGPSVFLFPPKPKDTLYITREPEVTCVVVDVSHEDPE | 4 |
| VKFNWYVDGVEVHNAKTKPREEQYNSTYRVVSVLTVLHQDWLNGKEYKC | |
| KVSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSP | |
| KTHTCPPCPAPELLGGPSVFLFPPKPKDTLYITREPEVTCVVVDVSHEDPEV | 5 |
| KFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSPGK | |
| KTHTCPPCPAPELLGGPSVFLFPPKPKDTLYITREPEVTCVVVDVSHEDPEV | 6 |
| KFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSPG | |
| KTHTCPPCPAPELLGGPSVFLFPPKPKDTLYITREPEVTCVVVDVSHEDPEV | 7 |
| KFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSP | |
| THTCPPCPAPELLGGPSVFLFPPKPKDTLYITREPEVTCVVVDVSHEDPEVKF | 8 |
| NWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSV | |
| MHEALKFHYTQKSLSLSPGK | |
| THTCPPCPAPELLGGPSVFLFPPKPKDTLYITREPEVTCVVVDVSHEDPEVKF | 9 |
| NWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSV | |
| MHEALKFHYTQKSLSLSPG | |
| THTCPPCPAPELLGGPSVFLFPPKPKDTLYITREPEVTCVVVDVSHEDPEVKF | 10 |
| NWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSV | |
| MHEALKFHYTQKSLSLSP | |
| TCPPCPAPELLGGPSVFLFPPKPKDTLYITREPEVTCVVVDVSHEDPEVKFN | 11 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPGK | |
| TCPPCPAPELLGGPSVFLFPPKPKDTLYITREPEVTCVVVDVSHEDPEVKFN | 12 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPG | |
| TCPPCPAPELLGGPSVFLFPPKPKDTLYITREPEVTCVVVDVSHEDPEVKFN | 13 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSP | |
| PPCPAPELLGGPSVFLFPPKPKDTLYITREPEVTCVVVDVSHEDPEVKFNWY | 14 |
| VDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIAVE | |
| WESNGQPENNYKTTPPVLDSDGSFFLYSKLTVDKSRWQQGNVFSCSVMHE | |
| ALKFHYTQKSLSLSPGK | |
| PPCPAPELLGGPSVFLFPPKPKDTLYITREPEVTCVVVDVSHEDPEVKFNWY | 15 |
| VDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIAVE | |
| WESNGQPENNYKTTPPVLDSDGSFFLYSKLTVDKSRWQQGNVFSCSVMHE | |
| ALKFHYTQKSLSLSPG | |
| PPCPAPELLGGPSVFLFPPKPKDTLYITREPEVTCVVVDVSHEDPEVKFNWY | 16 |
| VDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIAVE | |
| WESNGQPENNYKTTPPVLDSDGSFFLYSKLTVDKSRWQQGNVFSCSVMHE | |
| ALKFHYTQKSLSLSP | |
| HTCPPCPAPELLGGPSVFLFPPKPKDTLYITREPEVTCVVVDVSHEDPEVKFN | 17 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPGK | |
| HTCPPCPAPELLGGPSVFLFPPKPKDTLYITREPEVTCVVVDVSHEDPEVKFN | 18 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPG | |
| HTCPPCPAPELLGGPSVFLFPPKPKDTLYITREPEVTCVVVDVSHEDPEVKFN | 19 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSP | |
| CPPCPAPELLGGPSVFLFPPKPKDTLYITREPEVTCVVVDVSHEDPEVKFNW | 20 |
| YVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMH | |
| EALKFHYTQKSLSLSPG | |
| CPPCPAPELLGGPSVFLFPPKPKDTLYITREPEVTCVVVDVSHEDPEVKFNW | 21 |
| YVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMH | |
| EALKFHYTQKSLSLSPGK | |
| CPPCPAPELLGGPSVFLFPPKPKDTLYITREPEVTCVVVDVSHEDPEVKFNW | 22 |
| YVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMH | |
| EALKFHYTQKSLSLSP | |

In an embodiment, the first Fc domain and/or the second Fc domain comprise an amino acid sequence independently selected from the group consisting of SEQ ID NOs: 2, 3, 20, or 21. In an embodiment, the first Fc domain and the second Fc domain comprise an amino acid sequence independently selected from the group consisting of SEQ ID NOs: 2, 3, 20, or 21. In some embodiments, the FcRn antagonist comprises a population of FcRn antagonist molecules. In some embodiments, an FcRn antagonist comprising a first Fc domain and a second Fc domain comprising an amino acid sequence independently selected from the group consisting of SEQ ID NOs: 2, 3, 20, or 21 is the predominant FcRn antagonist molecule in the population of FcRn antagonist molecules. In some embodiments, the predominant FcRn antagonist molecule makes up at least 50%, 55%, 60%, 65%, 70%, 75%, 80%, 85%, 90%, 95%, 96%, 97%, 98%, or 99% of the population of FcRn antagonist molecules.

In an embodiment, the amino acid sequence of the Fc domains of the variant Fc region comprises the amino acid sequence of SEQ ID NO: 2. In an embodiment, the amino acid sequence of the Fc domains of the variant Fc region consists of the amino acid sequence of SEQ ID NO: 2. In an embodiment, the amino acid sequence of the Fc domains of the variant Fc region comprises the amino acid sequence of SEQ ID NO: 3. In an embodiment, the amino acid sequence of the Fc domains of the variant Fc region consists of the amino acid sequence of SEQ ID NO: 3. In an embodiment, the amino acid sequence of the Fc domains of the variant Fc region comprises the amino acid sequence of SEQ ID NO: 20. In an embodiment, the amino acid sequence of the Fc domains of the variant Fc region consists of the amino acid sequence of SEQ ID NO: 20. In an embodiment, the amino acid sequence of the Fc domains of the variant Fc region comprises the amino acid sequence of SEQ ID NO: 21. In an embodiment, the amino acid sequence of the Fc domains of the variant Fc region consists of the amino acid sequence of SEQ ID NO: 21.

In certain embodiments, the variant Fc region is a heterodimer, where the constituent Fc domains are different from each other. Methods of producing Fc heterodimers are known in the art (see, e.g., U.S. Pat. No. 8,216,805, which is incorporated by reference herein in its entirety). In an embodiment, the FcRn antagonist consists of a variant Fc region, wherein the variant Fc region consists of two Fc domains which form a heterodimer, wherein the amino acid sequence of each of the Fc domains is independently selected from SEQ ID NOs: 2, 3, 20, or 21. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 2 and the amino acid sequence of the second Fc domain consists of or comprises the amino acid sequence of SEQ ID NOs: 3, 20, or 21. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 3 and the amino acid sequence of the second Fc domain consists of or comprises the amino acid sequence of SEQ ID NOs: 2, 20, or 21. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 20 and the amino acid sequence of the second Fc domain consists of or comprises the amino acid sequence of SEQ ID NOs: 2, 3, or 21. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 21 and the amino acid sequence of the second Fc domain consists of or comprises the amino acid sequence of SEQ ID NOs: 2, 3, or 20.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 2.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 3.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 20.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 21.

In some embodiments, the FcRn antagonist comprises glycanation on one or both of the Fc domains. In some embodiments, the FcRn antagonist molecules comprise glycanation at EU position 297 on one or both of the Fc domains. In some embodiments, the glycanation comprises an N-glycan. In some embodiments, the N-glycan comprises a G0F N-glycan, G1F N-glycan, G2F N-glycan, or G0 N-glycan.

In some embodiments, FcRn antagonist comprises or consists of a population of FcRn antagonists, wherein at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, or at least 57% of the population of Fc domains of the FcRn antagonists comprise galactose. In some embodiments, the population comprises or consists of FcRn antagonists, wherein at least 88%, at least 89%, at least 90%, at least 91%, at least 92%, at least 93%, at least 94%, at least 95%, at least 96%, at least 97%, at least 98%, or at least 99% of the population of Fc domains of the FcRn antagonists comprise fucose.

In some embodiments, the FcRn antagonist lacks an amino acid at EU position 441 of one or both Fc domains. In some embodiments, the FcRn antagonist comprises glycine and lysine at EU positions 440 and 441, respectively. In some embodiments, the FcRn antagonist lacks amino acids at EU positions 440 and 441. In some embodiments, the FcRn antagonist comprises amidated proline at EU position 439. In some embodiments, the FcRn antagonist lacks amino acids at EU positions 440 and 441 and comprises amidated proline at EU position 439.

In some embodiments, the FcRn antagonist comprises aspartate, lysine, threonine, histidine, threonine, and cysteine at EU positions 221, 222, 223, 224, 225, and 226, respectively. In some embodiments, the FcRn antagonist lacks an amino acid at EU positions 221, and comprises lysine, threonine, histidine, threonine, and cysteine at EU positions 222, 223, 224, 225, and 226, respectively. In some embodiments, the FcRn antagonist lacks amino acids at EU positions 221 and 222, and comprises threonine, histidine, threonine, and cysteine at EU positions 223, 224, 225, and 226, respectively. In some embodiments, the FcRn antagonist lacks amino acids at EU positions 221-224, and comprises threonine and cysteine at EU positions 225 and 226, respectively. In some embodiments, the FcRn antagonist lacks amino acids at EU positions 221, 222, 223, 224, 225, and 226.

In some embodiments, the FcRn antagonist is a population of FcRn antagonist molecules. In some embodiments, the population of FcRn antagonist molecules comprises or consists of multiple subpopulations of FcRn antagonist molecules. In some embodiments, the population of FcRn antagonist molecules comprises or consists of 2, 3, 4, 5, 6, 7, 8, 9, 10, 11, 12, 13, 14, 15, 16, 17, 18, 19, or 20 subpopulations. In some embodiments, the population of FcRn antagonist molecules comprises or consists of 2, 3, 4, 5, 6, 7, 8, 9, 10, or 11 subpopulations.

In some embodiments, a first subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3.

In some embodiments, a second subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 12, respectively.

In some embodiments, a third subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 9, respectively.

In some embodiments, a fourth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein two asparagine residues in each FcRn antagonist molecule in the fourth subpopulation are deaminated.

In some embodiments, a fifth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 9, respectively, and wherein one asparagine residue in each FcRn antagonist molecule in the fifth subpopulation are deaminated.

In some embodiments, a fifth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein one asparagine residue in each FcRn antagonist molecule in the fifth subpopulation are deaminated.

In some embodiments, a sixth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 2 and 3, respectively.

In some embodiments, a seventh subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 2 and 3, respectively, and wherein one methionine residue or one tryptophan residue in each FcRn antagonist molecule in the seventh subpopulation is oxidized.

In some embodiments, a seventh subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein one methionine residue or one tryptophan residue in each FcRn antagonist molecule in the seventh subpopulation is oxidized.

In some embodiments, an eighth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 2.

In some embodiments, a ninth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 6, respectively.

In some embodiments, a tenth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 2 and 3, respectively, and wherein one methionine residue or one tryptophan residue in each FcRn antagonist molecule in the tenth subpopulation is oxidized.

In some embodiments, an eleventh subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein one methionine residue or one tryptophan residue in each FcRn antagonist molecule in the eleventh subpopulation is oxidized.

In some embodiments, an eleventh subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein two amino acid residues, independently selected from a methionine residue or a tryptophan residue, in each FcRn antagonist molecule in the eleventh subpopulation is oxidized.

In some embodiments, a first subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3.

In some embodiments, a second subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 12, respectively.

In some embodiments, a third subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 9, respectively.

In some embodiments, a fourth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3, and wherein two asparagine residues in each FcRn antagonist molecule in the fourth subpopulation are deaminated.

In some embodiments, a fifth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 9, respectively, and wherein one asparagine residue in each FcRn antagonist molecule in the fifth subpopulation is deaminated.

In some embodiments, a fifth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NO: 3, and wherein one asparagine residue in each FcRn antagonist molecule in the fifth subpopulation is deaminated.

In some embodiments, a sixth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 2 and 3, respectively.

In some embodiments, a seventh subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 2 and 3, respectively, and wherein one methionine residue or one tryptophan residue in each FcRn antagonist molecule in the seventh subpopulation is oxidized.

In some embodiments, a seventh subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NO: 3, and wherein one methionine residue or one tryptophan residue in each FcRn antagonist molecule in the seventh subpopulation is oxidized.

In some embodiments, an eighth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 2.

In some embodiments, a ninth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 6, respectively.

In some embodiments, a tenth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 2 and 3, respectively, and wherein one methionine residue or one tryptophan residue in each FcRn antagonist molecule in the tenth subpopulation is oxidized.

In some embodiments, an eleventh subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3, and wherein one methionine residue or one tryptophan residue in each FcRn antagonist molecule in the eleventh subpopulation is oxidized.

In some embodiments, an eleventh subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3, and wherein two amino acid residues, independently selected from a methionine residue or a tryptophan residue in each FcRn antagonist molecule in the eleventh subpopulation is oxidized.

In some embodiments, the population of FcRn antagonist molecules comprises or consists of the first subpopulation combined with one of the second, third, fourth, fifth, sixth, seventh, eighth, ninth, tenth, or eleventh subpopulations. In some embodiments, the population of FcRn antagonist molecules comprises or consists of the first subpopulation combined with two of the second, third, fourth, fifth, sixth, seventh, eighth, ninth, tenth, or eleventh subpopulations. In some embodiments, the population of FcRn antagonist molecules comprises or consists of the first subpopulation combined with three of the second, third, fourth, fifth, sixth, seventh, eighth, ninth, tenth, or eleventh subpopulations. In some embodiments, the population of FcRn antagonist molecules comprises or consists of the first subpopulation combined with four of the second, third, fourth, fifth, sixth, seventh, eighth, ninth, tenth, or eleventh subpopulations. In some embodiments, the population of FcRn antagonist molecules comprises or consists of the first subpopulation combined with five of the second, third, fourth, fifth, sixth, seventh, eighth, ninth, tenth, or eleventh subpopulations. In some embodiments, the population of FcRn antagonist molecules comprises or consists of the first subpopulation combined with six of the second, third, fourth, fifth, sixth, seventh, eighth, ninth, tenth, or eleventh subpopulations. In some embodiments, the population of FcRn antagonist molecules comprises or consists of the first subpopulation combined with seven of the second, third, fourth, fifth, sixth, seventh, eighth, ninth, tenth, or eleventh subpopulations. In some embodiments, the population of FcRn antagonist molecules comprises or consists of the first subpopulation combined with eight of the second, third, fourth, fifth, sixth, seventh, eighth, ninth, tenth, or eleventh subpopulations. In some embodiments, the population of FcRn antagonist molecules comprises or consists of the first subpopulation combined with nine of the second, third, fourth, fifth, sixth, seventh, eighth, ninth, tenth, or eleventh subpopulations. In some embodiments, the population of FcRn antagonist molecules comprises or consists of the first subpopulation combined with all of the second, third, fourth, fifth, sixth, seventh, eighth, ninth, tenth, or eleventh subpopulations.

In some embodiments, the population comprises or consists of the first and second subpopulations. In some embodiments, the population comprises or consists of the first and third subpopulations. In some embodiments, the population comprises or consists of the first and fourth subpopulations. In some embodiments, the population comprises or consists of the first and fifth subpopulations. In some embodiments, the population comprises or consists of the first and sixth subpopulations. In some embodiments, the population comprises or consists of the first and seventh subpopulations. In some embodiments, the population comprises or consists of the first and eighth subpopulations. In some embodiments, the population comprises or consists of the first and ninth subpopulations. In some embodiments, the population comprises or consists of the first and tenth subpopulations. In some embodiments, the population comprises or consists of the first and eleventh subpopulations. In some embodiments, the populations listed above further comprise or consist of 1, 2, 3, 4, 5, 6, 7, 8, or 9 additional subpopulations. In some embodiments, these additional subpopulations are one or more of those described above.

In some embodiments, the population comprises or consists of the first and seventh, ninth, or eleventh subpopulations. In some embodiments, the population comprises or consists of the first, seventh, ninth, and eleventh subpopulations.

In some embodiments, the first subpopulation makes up at least 40%, at least 45%, at least 50%, at least 55%, at least 60%, at least 65%, at least 70%, at least 75%, at least 80%, at least 85%, or at least 90% of the population of FcRn antagonist molecules. In some embodiments, the first subpopulation makes up about 40%, about 45%, about 50%, about 55%, about 60%, about 65%, about 70%, about 75%, about 80%, about 85%, or about 90% of the population of FcRn antagonist molecules. In some embodiments, the first subpopulation makes up 40%, 45%, 50%, 55%, 60%, 65%, 70%, 75%, 80%, 85%, or 90% of the population of FcRn antagonist molecules. In some embodiments, the first subpopulation makes up 40%-90%, 50%-80%, or 55%-70% of the population of FcRn antagonist molecules. In some embodiments, the first subpopulation makes up 56.9%-68.3% or 59.5%-67.9% of the population of FcRn antagonist molecules.

In some embodiments, the second subpopulation makes up less than 3.0%, less than 2.5%, less than 2.0%, less than 1.5%, less than 1%, or less than 0.5% of the population of FcRn antagonist molecules. In some embodiments, the second subpopulation makes up about 3.0%, about 2.5%, about 2.0%, about 1.5%, about 1%, or about 0.5% of the population of FcRn antagonist molecules. In some embodiments, the second subpopulation makes up 3.0%, 2.5%, 2.0%, 1.5%, 1%, or 0.5% of the population of FcRn antagonist molecules. In some embodiments, the second subpopulation makes up 0.5%-3.0%, 1.0%-2.5%, or 1.0%-2.0% of the population of FcRn antagonist molecules. In some embodiments, the second subpopulation makes up 0.8%-2.0% or 0.8%-2.1% of the population of FcRn antagonist molecules.

In some embodiments, the third subpopulation makes up less than 3.0%, less than 2.5%, less than 2.0%, less than 1.5%, less than 1%, or less than 0.5% of the population of FcRn antagonist molecules. In some embodiments, the third subpopulation makes up about 3.0%, about 2.5%, about 2.0%, about 1.5%, about 1%, or about 0.5% of the population of FcRn antagonist molecules. In some embodiments, the third subpopulation makes up 3.0%, 2.5%, 2.0%, 1.5%, 1%, or 0.5% of the population of FcRn antagonist molecules. In some embodiments, the third subpopulation makes up 0.5%-3.0%, 1.0%-2.5%, or 1.0%-2.0% of the population of FcRn antagonist molecules. In some embodiments, the third subpopulation makes up 1.1%-2.1% or 1.0%-1.9% of the population of FcRn antagonist molecules.

In some embodiments, the fourth subpopulation makes up less than 5%, less than 4%, less than 3%, less than 2%, or less than 1% of the population of FcRn antagonist molecules. In some embodiments, the fourth subpopulation makes up about 5%, about 4%, about 3%, about 2%, or about 1% of the population of FcRn antagonist molecules. In some embodiments, the fourth subpopulation makes up 5%, 4%, 3%, 2%, or 1% of the population of FcRn antagonist molecules. In some embodiments, the fourth subpopulation makes up 1%-5%, 2%-4%, or 2%-3% of the population of FcRn antagonist molecules. In some embodiments, the fourth subpopulation makes up 2.1%-3.2% or 2.0%-3.1% of the population of FcRn antagonist molecules.

In some embodiments, the fifth subpopulation makes up less than 12%, less than 11%, less than 10%, less than 9%, less than 8%, less than 7%, less than 6%, or less than 5% of the population of FcRn antagonist molecules. In some embodiments, the fifth subpopulation makes up about 12%, about 11%, about 10%, about 9%, about 8%, about 7%, about 6%, or about 5% of the population of FcRn antagonist molecules. In some embodiments, the fifth subpopulation makes up 12%, 11%, 10%, 9%, 8%, 7%, 6%, or 5% of the population of FcRn antagonist molecules. In some embodiments, the fifth subpopulation makes up 5%-12%, 6%-10%, or 7%-8% of the population of FcRn antagonist molecules. In some embodiments, the fifth subpopulation makes up 6.8%-9.4% or 6.9%-8.7% of the population of FcRn antagonist molecules.

In some embodiments, the sixth subpopulation makes up less than 17%, less than 16%, less than 15%, less than 14%, less than 13%, less than 12%, less than 11%, less than 10%, less than 9%, less than 8%, less than 7%, or less than 6% of the population of FcRn antagonist molecules. In some embodiments, the sixth subpopulation makes up about 17%, about 16%, about 15%, about 14%, about 13%, about 12%, about 11%, about 10%, about 9%, about 8%, about 7%, or about 6% of the population of FcRn antagonist molecules. In some embodiments, the sixth subpopulation makes up 17%, 16%, 15%, 14%, 13%, 12%, 11%, 10%, 9%, 8%, 7%, or 6% of the population of FcRn antagonist molecules. In some embodiments, the sixth subpopulation makes up 7%-17%, 10%-15%, or 11%-12% of the population of FcRn antagonist molecules. In some embodiments, the sixth subpopulation makes up 7.0%-14.0% or 10.0%-14.4% of the population of FcRn antagonist molecules.

In some embodiments, the seventh subpopulation makes up less than 6.0%, less than 5.5%, less than 5.0%, less than 4.5%, less than 4.0%, less than 3.5%, less than 3.0%, less than 2.5%, less than 2.0%, less than 1.5%, less than 1%, or less than 0.5% of the population of FcRn antagonist molecules. In some embodiments, the seventh subpopulation makes up about 6.0%, about 5.5%, about 5.0%, about 4.5%, about 4.0%, about 3.5%, about 3.0%, about 2.5%, about 2.0%, about 1.5%, about 1%, or about 0.5% of the population of FcRn antagonist molecules. In some embodiments, the seventh subpopulation makes up 6.0%, 5.5%, 5.0%, 4.5%, 4.0%, 3.5%, 3.0%, 2.5%, 2.0%, 1.5%, 1%, or 0.5% of the population of FcRn antagonist molecules. In some embodiments, the seventh subpopulation makes up 0.5%-5.5%, 1.0%-3.0%, or 1.5%-2.5% of the population of FcRn antagonist molecules. In some embodiments, the seventh subpopulation makes up 1.5%-5.5% or 1.4%-4.9% of the population of FcRn antagonist molecules.

In some embodiments, the eighth subpopulation makes up less than 7.5%, less than 7.0%, less than 6.5%, less than 6.0%, less than 5.5%, less than 5.0%, less than 4.5%, less than 4.0%, less than 3.5%, less than 3.0%, or less than 2.5% of the population of FcRn antagonist molecules. In some embodiments, the eighth subpopulation makes up about 7.5%, about 7.0%, about 6.5%, about 6.0%, about 5.5%, about 5.0%, about 4.5%, about 4.0%, about 3.5%, about 3.0%, or about 2.5% of the population of FcRn antagonist molecules. In some embodiments, the eighth subpopulation makes up 7.5%, 7.0%, 6.5%, 6.0%, 5.5%, 5.0%, 4.5%, 4.0%, 3.5%, 3.0%, or 2.5% of the population of FcRn antagonist molecules. In some embodiments, the eighth subpopulation makes up 2.5%-7.5%, 3.0%-5.0%, or 3.5%-4.5% of the population of FcRn antagonist molecules. In some embodiments, the eighth subpopulation makes up 2.9%-7.4% or 3.0%-6.3% of the population of FcRn antagonist molecules.

In some embodiments, the ninth subpopulation makes up less than 3.5%, less than 3.0%, less than 2.5%, less than 2.0%, less than 1.5%, less than 1%, or less than 0.5% of the population of FcRn antagonist molecules. In some embodiments, the ninth subpopulation makes up about 3.5%, about 3.0%, about 2.5%, about 2.0%, about 1.5%, about 1%, or about 0.5% of the population of FcRn antagonist molecules. In some embodiments, the ninth subpopulation makes up 3.5%, 3.0%, 2.5%, 2.0%, 1.5%, 1%, or 0.5% of the population of FcRn antagonist molecules. In some embodiments, the ninth subpopulation makes up 0.5%-3.5%, 1.5%-2.0%, or 1.0%-1.5% of the population of FcRn antagonist molecules. In some embodiments, the ninth subpopulation makes up 0.4%-3.2% or 0.5%-2.6% of the population of FcRn antagonist molecules.

In some embodiments, the tenth subpopulation makes up less than 2.0%, less than 1.5%, less than 1%, or less than 0.5% of the population of FcRn antagonist molecules. In some embodiments, the tenth subpopulation makes up about 2.0%, about 1.5%, about 1%, or about 0.5% of the population of FcRn antagonist molecules. In some embodiments, the tenth subpopulation makes up 2.0%, 1.5%, 1%, or 0.5% of the population of FcRn antagonist molecules. In some embodiments, the tenth subpopulation makes up 0.5%-2.0%, 0.5%-1.5%, or 1.0%-1.5% of the population of FcRn antagonist molecules.

In some embodiments, the eleventh subpopulation makes up less than 2.0%, less than 1.5%, less than 1%, or less than 0.5% of the population of FcRn antagonist molecules. In some embodiments, the eleventh subpopulation makes up about 2.0%, about 1.5%, about 1%, or about 0.5% of the population of FcRn antagonist molecules. In some embodiments, the eleventh subpopulation makes up 2.0%, 1.5%, 1%, or 0.5% of the population of FcRn antagonist molecules. In some embodiments, the eleventh subpopulation makes up 0.5%-2.0%, 0.5%-1.5%, or 1.0%-1.5% of the population of FcRn antagonist molecules.

In some embodiments, the population of FcRn antagonist molecules comprises one or more of the FcRn antagonists described herein. In some embodiments, the FcRn antagonist is any of those described in U.S. Patent Application No. 63/383,599, filed on Nov. 14, 2022, incorporated herein by reference in its entirety. In some embodiments, the FcRn antagonist is a population of FcRn antagonists as described in U.S. Patent Application No. 63/383,599, filed on Nov. 14, 2022, incorporated herein by reference in its entirety.

In an embodiment, the FcRn antagonist is efgartigimod (CAS Registry No. 1821402-21-4). The term “efgartigimod” as used herein is interchangeable with “ARGX-113” and “efgartigimod alfa.” In some embodiments, efgartigimod is efgartigimod alfa-fcab.

In an embodiment, the anti-FcRn antibody is nipocalimab (M281), rozanolixizumab (UCB7665), orilanolimab (ALXN1830/SYNT001), or batoclimab (IMVT-1401/RVT1401/HBM9161).

In an embodiment, an antibody that binds specifically to FcRn and inhibits the binding of the Fc region of immunoglobulin to FcRn is nipocalimab, also known as M281. Nipocalimab is a full-length “Fc dead” IgG1 monoclonal antibody. Nipocalimab has been administered as an intravenous infusion in Phase 2 clinical trials for the treatment of myasthenia gravis (MG), warm autoimmune hemolytic anemia (WAIHA), and hemolytic disease of fetus and newborn (HDFN). Nipocalimab comprises the light chain (SEQ ID NO: 23) and heavy chain (SEQ ID NO: 24) sequences set forth in Table 3 below (VL region of SEQ ID NO: 23 and VH region of SEQ ID NO: 24 are underlined):

TABLE 3
Heavy chain and light chain sequences of nipocalimab

| SEQ ID NO: | Amino Acid Sequence |
|---|---|
| 23 | QSALTQPASVSGSPGQSITISCTGTGSDVGSYNLVSWYQQHPGKAP |
| (light chain | KLMIYGDSERPSGVSNRFSGSKSGNTASLTISGLQAEDEADYYCSSY |
| sequence; | AGSGIYVFGTGTKVTVLGQPKAAPSVTLFPPSSEELQANKATLVCLI |
| VL region | SDFYPGAVTVAWKADSSPVKAGVETTTPSKQSNNKYAASSYLSLT |
| underlined) | PEQWKSHKSYSCQVTHEGSTVEKTVAPTECS |
| 24 | EVQLLESGGGLVQPGGSLRLSCAASGFTFSTYAMGWVRQAPGKGL |
| (heavy chain | EWVSSIGASGSQTRYADSVKGRFTISRDNSKNTLYLQMNSLRAEDT |
| sequence; | AVYYCARLAIGDSYWGQGTMVTVSSASTKGPSVFPLAPSSKSTSGG |
| VH region | TAALGCLVKDYFPEPVTVSWNSGALTSGVHTFPAVLQSSGLYSLSS |
| underlined) | VVTVPSSSLGTQTYICNVNHKPSNTKVDKKVEPKSCDKTHTCPPCP |
| | APELLGGPSVFLFPPKPKDTLMISRTPEVTCVVVDVSHEDPEVKFN |
| | WYVDGVEVHNAKTKPREEQYASTYRVVSVLTVLHQDWLNGKEY |
| | KCKVSNKALPAPIEKTISKAKGQPREPQVYTLPPSREEMTKNQVSLT |
| | CLVKGFYPSDIAVEWESNGQPENNYKTTPPVLDSDGSFFLYSKLTV |
| | DKSRWQQGNVFSCSVMHEALHNHYTQKSLSLSPG |

In an embodiment, an antibody that binds specifically to FcRn and inhibits the binding of the Fc region of immunoglobulin to FcRn is rozanolixizumab, also known as UCB 7665. Rozanolixizumab is a full-length humanized IgG4 monoclonal antibody. Rozanolixizumab has been administered as a subcutaneous infusion in ongoing clinical trials for MG, immune thrombocytopenia (ITP), and chronic inflammatory demyelinating polyneuropathy (CIDP). Rozanolixizuab comprises the light chain (SEQ ID NO: 25) and heavy chain (SEQ ID NO: 26) sequences set forth in Table 4 below (VL region of SEQ ID NO: 25 and VH region of SEQ ID NO: 26 are underlined):

TABLE 4
Heavy chain and light chain sequences of rozanolixizumab

| SEQ ID NO: | Amino Acid Sequence |
|---|---|
| 25 | DIQMTQSPSSLSASVGDRVTITCKSSQSLVGASGKTYLYWLFQKPG |
| (light chain | KAPKRLIYLVSTLDSGIPSRFSGSGSGTEFTLTISSLQPEDFATYYCLQ |
| sequence; | GTHFPHTFGQGTKLEIKRTVAAPSVFIFPPSDEQLKSGTASVVCLLN |
| VL region | NFYPREAKVQWKVDNALQSGNSQESVTEQDSKDSTYSLSSTLTLS |
| underlined) | KADYEKHKVYACEVTHQGLSSPVTKSFNRGEC |
| 26 | EVPLVESGGGLVQPGGSLRLSCAVSGFTFSNYGMVWVRQAPGKGL |
| (heavy chain | EWVAYIDSDGDNTYYRDSVKGRFTISRDNAKSSLYLQMNSLRAED |
| sequence; | TAVYYCTTGIVRPFLYWGQGTLVTVSSASTKGPSVFPLAPCSRSTSE |
| VH region | STAALGCLVKDYFPEPVTVSWNSGALTSGVHTFPAVLQSSGLYSLS |
| underlined) | SVVTVPSSSLGTKTYTCNVDHKPSNTKVDKRVESKYGPPCPPCPAP |
| | EFLGGPSVFLFPPKPKDTLMISRTPEVTCVVVDVSQEDPEVQFNWY |
| | VDGVEVHNAKTKPREEQFNSTYRVVSVLTVLHQDWLNGKEYKCK |
| | VSNKGLPSSIEKTISKAKGQPREPQVYTLPPSQEEMTKNQVSLTCLV |
| | KGFYPSDIAVEWESNGQPENNYKTTPPVLDSDGSFFLYSRLTVDKS |
| | RWQEGNVFSCSVMHEALHNHYTQKSLSLSLGK |

In an embodiment, an antibody that binds specifically to FcRn and inhibits the binding of the Fc region of immunoglobulin to FcRn is orilanolimab, also known as SYNT001. Orilanolimab is another full-length humanized IgG4 monoclonal antibody. Orilanolimab has been administered as an intravenous infusion in Phase 2 clinical trials for treatment of WAIHA. Orilanolimab comprises the light chain (SEQ ID NO: 27) and heavy chain (SEQ ID NO: 28) sequences set forth in Table 5 below (VL region of SEQ ID NO: 27 and VH region of SEQ ID NO: 28 are underlined):

TABLE 5
Heavy chain and light chain sequences of orilanolimab

| SEQ ID NO: | Amino Acid Sequence |
|---|---|
| 27 | DIQMTQSPSSLSASVGDRVTITCKASDHINNWLAWYQQKPGQAPR |
| (light chain | LLISGATSLETGVPSRFSGSGTGKDYTLTISSLQPEDFATYYCQQYW |
| sequence; | STPYTFGGGTKVEIKRTVAAPSVFIFPPSDEQLKSGTASVVCLLNNF |
| VL region | YPREAKVQWKVDNALQSGNSQESVTEQDSKDSTYSLSSTLTLSKA |
| underlined) | DYEKHKVYACEVTHQGLSSPVTKSFNRGEC |
| 28 | QVQLVQSGAELKKPGASVKLSCKASGYTFTSYGISWVKQATGQGL |
| underlined) | EWIGEIYPRSGNTYYNEKFKGRATLTADKSTSTAYMELRSLRSEDS |
| sequence; | AVYFCARSTTVRPPGIWGTGTTVTVSSASTKGPSVFPLAPCSRSTSE |
| (heavy chain | STAALGCLVKDYFPEPVTVSWNSGALTSGVHTFPAVLQSSGLYSLS |
| VH | SVVTVPSSSLGTKTYTCNVDHKPSNTKVDKRVESKYGPPCPPCPAP |
| region | EFLGGPSVFLFPPKPKDTLMISRTPEVTCVVVDVSQEDPEVQFNWY |
| | VDGVEVHNAKTKPREEQFNSTYRVVSVLTVLHQDWLNGKEYKCK |
| | VSNKGLPSSIEKTISKAKGQPREPQVYTLPPSQEEMTKNQVSLTCLV |
| | KGFYPSDIAVEWESNGQPENNYKTTPPVLDSDGSFFLYSRLTVDKS |
| | RWQEGNVFSCSVMHEALHNHYTQKSLSLSLG |

In an embodiment, an antibody that binds specifically to FcRn and inhibits the binding of the Fc region of immunoglobulin to FcRn is batoclimab, also known as IMVT1401/RVT1401/HBM9161. Batoclimab is another full-length “Fc dead” IgG1 monoclonal antibody. Batoclimab has been administered as a subcutaneous injection in ongoing Phase 2 clinical trials for treatment of MG and Graves' ophthalmopathy. Batoclimab comprises the light chain (SEQ ID NO: 29) and heavy chain (SEQ ID NO: 30) sequences set forth in Table 6 below (VL region of SEQ ID NO: 29 and VH region of SEQ ID NO: 30 are underlined):

TABLE 6
Heavy chain and light chain sequences of batoclimab

| SEQ ID NO: | Amino Acid Sequence |
|---|---|
| 29 | SYVLTQSPSVSVAPGQTARITCGGNNIGSKSVHWYQQKPGQAPVL |
| (light chain | VVYDDSDRPSGIPERFSASNSGNTATLTISRVEAGDEADYYCQVWD |
| sequence; | SSSDHVVFGGGTKLTVLGQPKAAPSVTLFPPSSEELQANKATLVCLI |
| VL region | SDFYPGAVTVAWKADSSPVKAGVETTTPSKQSNNKYAASSYLSLT |
| underlined) | PEQWKSHRSYSCQVTHEGSTVEKTVAPTECS |
| 30 | QLLLQESGPGLVKPSETLSLTCTVSGGSLSSSFSYWVWIRQPPGKGL |
| (heavy chain | EWIGTIYYSGNTYYNPSLKSRLTISVDTSKNHFSLKLSSVTAADTAV |
| sequence; | YYCARRAGILTGYLDSWGQGTLVTVSSASTKGPSVFPLAPSSKSTS |
| VH region | GGTAALGCLVKDYFPEPVTVSWNSGALTSGVHTFPAVLQSSGLYS |
| underlined) | LSSVVTVPSSSLGTQTYICNVNHKPSNTKVDKRVEPKSCDKTHTCP |
| | PCPAPEAAGGPSVFLFPPKPKDTLMISRTPEVTCVVVDVSHEDPEVK |
| | FNWYVDGVEVHNAKTKPREEQYNSTYRVVSVLTVLHQDWLNGK |
| | EYKCKVSNKALPAPIEKTISKAKGQPREPQVYTLPPSREEMTKNQV |
| | SLTCLVKGFYPSDIAVEWESNGQPENNYKTTPPVLDSDGSFFLYSK |
| | LTVDKSRWQQGNVFSCSVMHEALHNHYTQKSLSLSPG |

### Pharmaceutical Compositions

In an aspect, the instant disclosure provides pharmaceutical compositions comprising an FcRn antagonist for use in methods described herein. In certain embodiments, these compositions comprise or consist of a variant Fc region, or FcRn binding fragment thereof, that binds specifically to FcRn, particularly human FcRn, with increased affinity and reduced pH dependence relative to a native Fc region. In other embodiments, the FcRn antagonist composition is an antibody or antigen-binding fragment thereof that binds specifically to FcRn via its antigen binding domain and inhibits the binding of Fc region of immunoglobulin to FcRn. In general, these FcRn antagonists inhibit the binding of Fc-containing agents (e.g., antibodies and immunoadhesins) to FcRn in vivo, which results in an increased rate of degradation of the Fc-containing agents and, concomitantly, a reduced serum level of these agents.

In an embodiment, the FcRn antagonist is efgartigimod. Efgartigimod (ARGX-113) is a modified human immunoglobulin (Ig) gamma (IgG) 1-derived Fc of the za allotype that binds with nanomolar affinity to human FcRn. Efgartigimod encompasses the IgG1 Fc-region and has been engineered using ABDEG™ technology to increase its affinity for FcRn at both physiological and acidic pH. The increased affinity for FcRn of efgartigimod at both acidic and physiological pH results in a blockage of FcRn-mediated recycling of IgG.

Due to its increased affinity for FcRn at both acidic and neutral pH, efgartigimod blocks the FcRn/IgG complex from forming, which results in degradation of endogenous IgGs, including autoantibodies that cause IgG-mediated autoimmune diseases. This blocking of FcRn by efgartigimod results in a rapid and profound reduction in autoantibody levels, which underlies the therapeutic strategy for the treatment of autoimmune indications where IgG autoantibodies are expected to have a central role in the disease pathology.

Efgartigimod is under development for both the intravenous (IV) and subcutaneous (SC) administration route.

For IV administration, in certain embodiments, efgartigimod may be administered in a formulation comprising sodium phosphate, sodium chloride, L-arginine hydrochloride, and polysorbate 80. In certain embodiments, efgartigimod may be administered in a formulation comprising about 25 mM sodium phosphate, about 100 mM sodium chloride, and about 150 mM L-arginine hydrochloride (pH 6.7), with about 0.02% (w/v) polysorbate 80. In certain embodiments, efgartigimod may be administered in a formulation comprising 25 mM sodium phosphate, 100 mM sodium chloride, and 150 mM L-arginine hydrochloride (pH 6.7), with 0.02% (w/v) polysorbate 80. In certain embodiments, efgartigimod may be administered in a formulation comprising about 25 mM sodium phosphate, about 100 mM sodium chloride, and about 150 mM L-arginine hydrochloride (pH 6.7), with about 0.02% (w/v) polysorbate 80, via intravenous infusion in a total volume of about 250 mL over a period of about 2 hours. In certain embodiments, efgartigimod may be administered in a formulation comprising 25 mM sodium phosphate, 100 mM sodium chloride, and 150 mM L-arginine hydrochloride (pH 6.7), with 0.02% (w/v) polysorbate 80, via intravenous infusion in a total volume of 250 mL over a period of 2 hours. See, e.g., WO2019110823A1, which is incorporated by reference herein in its entirety.

In certain embodiments, efgartigimod may be administered in a formulation comprising an aqueous solution comprising about 25 mM sodium phosphate, about 100 mM sodium chloride, and about 150 mM L-arginine hydrochloride with a pH of about 6.7, with about 0.02% (w/v) polysorbate 80, diluted for intravenous infusion to a total volume of about 125 mL over a period of about 1 hour. In certain embodiments, efgartigimod may be administered in a formulation comprising an aqueous solution comprising 25 mM sodium phosphate, 100 mM sodium chloride, and 150 mM L-arginine hydrochloride with a pH of 6.7, with 0.02% (w/v) polysorbate 80, diluted for intravenous infusion to a total volume of 125 mL over a period of 1 hour.

In certain embodiments, efgartigimod may be administered in a formulation comprising an aqueous solution comprising about 4 mM sodium phosphate, about 146 mM sodium chloride, about 24 mM L-arginine, and about 0.0032% (w/v) polysorbate 80, with a pH of about 6.7. This formulation is administered via intravenous infusion in a total volume of about 125 mL over a period of about 1 hour. In certain embodiments, efgartigimod may be administered in a formulation comprising an aqueous solution comprising 4 mM sodium phosphate, 146 mM sodium chloride, 24 mM L-arginine, and 0.0032% (w/v) polysorbate 80, with a pH of 6.7. This formulation is administered via intravenous infusion in a total volume of 125 mL over a period of 1 hour.

In certain embodiments, efgartigimod is administered via IV infusion and is provided in a sterile, colorless, clear concentrate solution at a concentration of about 20 mg/mL. In certain embodiments, efgartigimod is administered via IV infusion and is provided in a sterile, colorless, clear concentrate solution at a concentration of 20 mg/mL.

In certain embodiments, efgartigimod is administered via IV infusion and is provided in a vial (e.g., a single-dose vial). In certain embodiments, a vial of efgartigimod contains about 400 mg of efgartigimod at a concentration of about 20 mg/mL. In certain embodiments, a vial of efgartigimod contains 400 mg of efgartigimod at a concentration of 20 mg/mL. In certain embodiments, each mL of solution in a vial of efgartigimod contains about 31.6 mg L-arginine hydrochloride, about 0.2 mg polysorbate 80, about 5.8 mg sodium chloride, about 2.4 mg sodium phosphate dibasic anhydrous, about 1.1 mg sodium phosphate monobasic monohydrate, and water for injection, USP, at a pH of about 6.7. In certain embodiments, each mL of solution in a vial of efgartigimod contains 31.6 mg L-arginine hydrochloride, 0.2 mg polysorbate 80, 5.8 mg sodium chloride, 2.4 mg sodium phosphate dibasic anhydrous, 1.1 mg sodium phosphate monobasic monohydrate, and water for injection, USP, at a pH of 6.7.

In certain embodiments, for patients weighing under 120 kg, efgartigimod is administered at a dose of about 10 mg/kg as an IV infusion. In certain embodiments, for patients weighing under 120 kg, efgartigimod is administered at a dose of about 10 mg/kg as an IV infusion over about 1 hour. In certain embodiments, for patients weighing under 120 kg, efgartigimod is administered at a dose of about 10 mg/kg as an IV infusion over about 1 hour once weekly. In certain embodiments, for patients weighing under 120 kg, efgartigimod is administered at a dose of about 10 mg/kg as an IV infusion over about 1 hour once weekly for about 4 weeks. In certain embodiments, for patients weighing under 120 kg, efgartigimod is administered at a dose of 10 mg/kg as an IV infusion. In certain embodiments, for patients weighing under 120 kg, efgartigimod is administered at a dose of 10 mg/kg as an IV infusion over 1 hour. In certain embodiments, for patients weighing under 120 kg, efgartigimod is administered at a dose of 10 mg/kg as an IV infusion over 1 hour once weekly. In certain embodiments, for patients weighing under 120 kg, efgartigimod is administered at a dose of 10 mg/kg as an IV infusion over 1 hour once weekly for 4 weeks. In certain embodiments, for patients weighing 120 kg or more, efgartigimod is administered at a dose of about 1200 mg per IV infusion. In certain embodiments, for patients weighing 120 kg or more, efgartigimod is administered at a dose of 1200 mg per IV infusion.

For SC administration, in certain embodiments, efgartigimod may be administered alone. Alternatively, for SC administration, in certain embodiments, efgartigimod may be administered co-formulated with hyaluronidase, for example, rHuPH20. The co-formulated material will allow SC dosing of larger volumes.

In some embodiments, efgartigimod may be administered in a formulation comprising an aqueous solution comprising about 20 mM L-histidine, about 100 mM sodium chloride, about 60 mM sucrose, about 10 mM L-methionine, and about 0.04% (w/v) polysorbate 20, wherein the formulation has a pH of about 6.0. In some embodiments, the formulation comprises about 180 mg/mL efgartigimod. In some embodiments, efgartigimod may be administered in a formulation comprising an aqueous solution comprising 20 mM L-histidine, 100 mM sodium chloride, 60 mM sucrose, 10 mM L-methionine, and 0.04% (w/v) polysorbate 20, wherein the formulation has a pH of 6.0. In some embodiments, the formulation comprises 180 mg/mL efgartigimod.

In some embodiments, efgartigimod may be administered in a formulation comprising an aqueous solution comprising about 20 mM L-histidine, about 50 mM L-arginine, about 100 mM sodium chloride, about 60 mM sucrose, about 10 mM L-methionine, and about 0.04 (w/v) polysorbate 80, wherein the formulation has a pH of about 6.0. In some embodiments, the formulation comprises about 200 mg/mL efgartigimod. In some embodiments, efgartigimod may be administered in a formulation comprising an aqueous solution comprising 20 mM L-histidine, 50 mM L-arginine, 100 mM sodium chloride, 60 mM sucrose, 10 mM L-methionine, and 0.04 (w/v) polysorbate 80, wherein the formulation has a pH of 6.0. In some embodiments, the formulation comprises 200 mg/mL efgartigimod.

rHuPH20 is the active ingredient of Halozyme's commercial product HYLENEX® recombinant (hyaluronidase human injection), referred to as HYLENEX®, which was approved by FDA for marketed use in the U.S. in December 2005. HYLENEX® is a tissue permeability modifier indicated as an adjuvant in SC fluid administration for achieving hydration, to increase the dispersion and absorption of other injected drugs, and in SC urography, for improving resorption of radiopaque agents.

rHuPH20 is a recombinant enzyme human hyaluronidase produced by genetically engineered Chinese hamster ovary (CHO) cells containing a deoxyribonucleic plasmid encoding a soluble fragment of human hyaluronidase (posterior head protein 20 [PH20]).

The HZ202 rHuPH20 DS is currently registered in HYLENEX® and other biologic drug products co-formulated with rHuPH20 DS. As such, in certain embodiments HZ202 rHuPH20 DS is used in the efgartigimod/rHuPH20 co-formulated product for SC administration (i.e., efgartigimod PH20 SC).

Provided in the co-formulations, combinations, uses and methods herein are soluble hyaluronidases. Soluble hyaluronidases include any that, upon expression, are secreted from a cell and exist in soluble form. Such soluble hyaluronidases include, but are not limited to, bacterial soluble hyaluronidases, non-human soluble hyaluronidases, such as bovine PH20 and ovine PH20, human soluble PH20, and variants thereof. Generally soluble forms of PH20 are produced using protein expression systems that facilitate correct N-glycosylation to ensure the polypeptide retains activity, since glycosylation is important for the catalytic activity and stability of hyaluronidases. Such cells include, for example Chinese Hamster Ovary (CHO) cells (e.g., DG44 CHO cells).

In some embodiments, rHuPH20 refers to the composition produced upon expression in a cell, such as a CHO cell, of nucleic acid encoding residues 36-482 of SEQ ID NO: 32, generally linked to the native or a heterologous signal sequence (residues 1-35 of SEQ ID NO: 32). rHuPH20 is produced by expression of a nucleic acid molecule, such as encoding amino acids 1-482 (set forth in SEQ ID NO: 32) in a mammalian cell. Translational processing removes the 35 amino acid signal sequence. As produced in the culture medium there is heterogeneity at the C-terminus such that the product, designated rHuPH20, includes a mixture of species that can include any one or more of the polypeptides 36-480, 36-481, and 36-482 of SEQ ID NO: 32, and some shorter polypeptides, in various abundance. Typically, rHuPH20 is produced in cells that facilitate correct N-glycosylation to retain activity, such as CHO cells (e.g., DG44 CHO cells). In some embodiments, one of the most abundant species is the 446 amino acid polypeptide corresponding to residues 36-481 of SEQ ID NO: 32. In some embodiments, rHuPH20 refers to polypeptides that are soluble or secreted upon expression in a mammalian cell and have at least 90%, 91%, 92%, 93%, 94%, 95%, 96%, 97%, 98%, 99%, or more sequence identity with residues 36-482 of SEQ ID NO: 32. In some embodiments, the rHuPH20 is the 447 amino acid polypeptide of SEQ ID NO: 33.

TABLE 7
Exemplary hyaluronidase sequences

| SEQ ID NO: | Amino Acid Sequence |
|---|---|
| 32 | MGVLKFKHIFFRSFVKSSGVSQIVFTFLLIPCCLTLNFRAPPVIPNVPF |
| | LWAWNAPSEFCLGKFDEPLDMSLFSFIGSPRINATGQGVTIFYVDRL |
| | GYYPYIDSITGVTVNGGIPQKISLQDHLDKAKKDITFYMPVDNLGM |
| | AVIDWEEWRPTWARNWKPKDVYKNRSIELVQQQNVQLSLTEATE |
| | KAKQEFEKAGKDFLVETIKLGKLLRPNHLWGYYLFPDCYNHHYKK |
| | PGYNGSCFNVEIKRNDDLSWLWNESTALYPSIYLNTQQSPVAATLY |
| | VRNRVREAIRVSKIPDAKSPLPVFAYTRIVFTDQVLKFLSQDELVYT |
| | FGETVALGASGIVIWGTLSIMRSMKSCLLLDNYMETILNPYIINVTL |
| | AAKMCSQVLCQEQGVCIRKNWNSSDYLHLNPDNFAIQLEKGGKFT |
| | VRGKPTLEDLEQFSEKFYCSCYSTLSCKEKADVKDTDAVDVCIADG |
| | VCIDAFLKPPMETEEPQIFYNASPSTLSATMFIVSILFLIISSVASL |
| 33 | LNFRAPPVIPNVPFLWAWNAPSEFCLGKFDEPLDMSLFSFIGSPRIN |
| | ATGQGVTIFYVDRLGYYPYIDSITGVTVNGGIPQKISLQDHLDKAK |
| | KDITFYMPVDNLGMAVIDWEEWRPTWARNWKPKDVYKNRSIELV |
| | QQQNVQLSLTEATEKAKQEFEKAGKDFLVETIKLGKLLRPNHLWG |
| | YYLFPDCYNHHYKKPGYNGSCPNVEIKRNDDLSWLWNESTALYPS |
| | IYLNTQQSPVAATLYVRNRVREAIRVSKIPDAKSPLPVFAYTRIVFT |
| | DQVLKFLSQDELVYTFGETVALGASGIVIWGTLSIMRSMKSCLLLD |
| | NYMETILNPYIINVTLAAKMCSQVLCQEQGVCIRKNWNSSDYLHL |
| | NPDNFAIQLEKGGKFTVRGKPTLEDLEQFSEKFYCSCYSTLSCKEK |
| | ADVKDTDAVDVCIADGVCIDAFLKPPMETEEPQIFY |

In some embodiments, the pharmaceutical formulation comprises an FcRn antagonist in an amount from about 20 mg to about 20,000 mg. In some embodiments, the pharmaceutical formulation comprises an FcRn antagonist in an amount from about 200 mg to about 20,000 mg. In some embodiments, the pharmaceutical formulation comprises an FcRn antagonist in an amount from about 300 mg to about 6000 mg. In some embodiments, the pharmaceutical formulation comprises an FcRn antagonist in an amount from about 750 mg to about 3000 mg. In some embodiments, the pharmaceutical formulation comprises an FcRn antagonist in an amount from about 1000 mg to about 2500 mg. In some embodiments, the pharmaceutical formulation comprises an FcRn antagonist in an amount from about 1000 mg to about 2000 mg.

In some embodiments, the pharmaceutical formulation comprises an FcRn antagonist in an amount from 20 mg to 20,000 mg. In some embodiments, the pharmaceutical formulation comprises an FcRn antagonist in an amount from 200 mg to 20,000 mg. In some embodiments, the pharmaceutical formulation comprises an FcRn antagonist in an amount from 300 mg to 6000 mg. In some embodiments, the pharmaceutical formulation comprises an FcRn antagonist in an amount from 750 mg to 3000 mg. In some embodiments, the pharmaceutical formulation comprises an FcRn antagonist in an amount from 1000 mg to 2500 mg. In some embodiments, the pharmaceutical formulation comprises an FcRn antagonist in an amount from 1000 mg to 2000 mg.

In some embodiments, the pharmaceutical formulation comprises about 1000 mg or about 2000 mg of an FcRn antagonist. In some embodiments, the pharmaceutical formulation comprises 1000 mg or 2000 mg of an FcRn antagonist. In some embodiments, the FcRn antagonist is efgartigimod.

In some embodiments, the pharmaceutical formulation comprises efgartigimod in an amount from about 800 mg to about 1200 mg. In some embodiments, the pharmaceutical formulation comprises efgartigimod in an amount from 800 mg to 1200 mg.

In some embodiments, the pharmaceutical formulation comprises about 1000 mg efgartigimod. In some embodiments, the pharmaceutical formulation comprises 1000 mg efgartigimod.

In some embodiments, the pharmaceutical formulation comprises from about 10 mg/mL to about 200 mg/mL efgartigimod. In some embodiments, the pharmaceutical formulation comprises from 10 mg/mL to 200 mg/mL efgartigimod.

In some embodiments, the pharmaceutical formulation comprises about 20 mg/mL efgartigimod. In some embodiments, the pharmaceutical formulation comprises 20 mg/mL efgartigimod.

In some embodiments, the pharmaceutical formulation comprises about 180 mg/mL efgartigimod. In some embodiments, the pharmaceutical formulation comprises 180 mg/mL efgartigimod.

In some embodiments, the pharmaceutical formulation further comprises hyaluronidase. In some embodiments, the hyaluronidase is recombinant human hyaluronidase PH20 (rHuPH20).

The hyaluronidase can be present in the pharmaceutical formulation in any suitable amount. In an embodiment, the amount of hyaluronidase enzyme is from about 1000 U/mL to about 3000 U/mL. In an embodiment, the amount of hyaluronidase enzyme is about 1000 U/mL, about 1500 U/mL, about 2000 U/mL, about 2500 U/mL, or about 3000 U/mL. In an embodiment, the amount of hyaluronidase enzyme is 2000 U/mL.

In some embodiments, the rHuPH20 is present in the pharmaceutical formulation in an amount of about 11,000 U. In some embodiments, the rHuPH20 is present in the pharmaceutical formulation in an amount of 11,000 U.

In some embodiments, the pharmaceutical formulation comprises at least about 5 U to at least about 100,000 U of an endoglycosidase hydrolase enzyme. In some aspects, the pharmaceutical formulation comprises at least about 5 U, at least about 10 U, at least about 20 U, at least about 30 U, at least about 40 U, at least about 50 U, at least about 75 U, at least about 100 U, at least about 200 U, at least about 300 U, at least about 400 U, at least about 500 U, at least about 750 U, at least about 1000 U, at least about 2000 U, at least about 3000 U, at least about 4000 U, at least about 5000 U, at least about 6000 U, at least about 7000 U, at least about 8000 U, at least about 9000 U, at least about 10,000 U, at least about 20,000 U, at least about 30,000 U, at least about 40,000 U, at least about 50,000 U, at least about 60,000 U, at least about 70,000 U, at least about 80,000 U, at least about 90,000 U, or at least about 100,000 U of an endoglycosidase hydrolase enzyme.

In some embodiments, the pharmaceutical formulation comprises about 20,000 U of an endoglycosidase hydrolase enzyme. In some embodiments, the pharmaceutical formulation comprises at least about 500 U/mL to at least about 5000 U/mL of an endoglycosidase hydrolase enzyme. In some embodiments, the pharmaceutical formulation comprises at least about 1500 U/mL, at least about 1600 U/mL, at least about 1700 U/mL, at least about 1800 U/mL, at least about 1900 U/mL, at least about 2000 U/mL, at least about 2100 U/mL, at least about 2200 U/mL, at least about 2300 U/mL, at least about 2400 μM, at least about 2500 μM, at least about 3000 μM, at least about 3500 μM, at least about 4000 μM, at least about 4500 U/mL, or at least about 5000 U/mL of an endoglycosidase hydrolase enzyme. In some embodiments, the pharmaceutical formulation comprises about 2000 U/mL of an endoglycosidase hydrolase enzyme.

In some embodiments, the endoglycosidase hydrolase enzyme cleaves hyaluronic acid at a hexosaminidic β (1-4) or (1-3) linkage. In some embodiments, the endoglycosidase hydrolase enzyme comprises a catalytic domain of hyaluronidase PH-20 (HuPH20), HYAL1, HYAL2, HYAL3, HYAL4, or HYALPS1. In some embodiments, the endoglycosidase hydrolase enzyme comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to amino acids 36-490 of SEQ ID NO: 32. In some embodiments, the endoglycosidase hydrolase enzyme comprises a hyaluronidase. In some embodiments, the endoglycosidase hydrolase enzyme comprises a hyaluronidase selected from the group consisting of HuPH20, HYAL1, HYAL2, HYAL3, HYAL4, any variant, and any isoform thereof. In some embodiments, the endoglycosidase hydrolase enzyme comprises rHuPH20 or a fragment thereof.

In some embodiments, the endoglycosidase hydrolase enzyme comprises a modified hyaluronidase comprising one or more amino acid substitutions relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified hyaluronidase comprising one or more amino acid substitution in an alpha-helix region relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified hyaluronidase comprising one or more amino acid substitution in linker region relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified hyaluronidase, wherein one or more N-terminal and/or C-terminal amino acids are deleted relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified rHuPH20, wherein the modified rHuPH20 comprises: i. one or more amino acid substitution in an alpha-helix region, a linker region, or both an alpha-helix region and a linker region relative to wild-type rHuPH20; ii. deletion of one or more N-terminal amino acid, one or more C-terminal amino acid, or one or more N-terminal amino acid and one or more C-terminal amino acid relative to wild-type rHuPH20; or iii. both (i) and (ii).

“Hyaluronidase,” as used herein, refers to an enzyme capable of catalyzing the cleavage of hyaluronan. Hyaluronan is a repeating polymer of N-acetyl-glucosamine and glucuronic acid, which is present in the subcutaneous space and contributes to the soluble gel-like component of the extracellular matrix of the skin and is restored by rapid turnover (resynthesis). In some embodiments, the hyaluronidase comprises rHuPH20, which is a glycosylated 447-amino acid single chain polypeptide that depolymerizes hyaluronan in the subcutaneous space locally at the site of injection in the skin. Depolymerization of hyaluronan by hyaluronidase is accomplished by hydrolysis of the polysaccharide polymer. Depolymerization of hyaluronan results in a transient reduction in the viscosity of the gel-like phase of the extracellular matrix and increased hydraulic conductance that facilitates the dispersion and absorption of the coadministered therapeutic agent. Thus, a hyaluronidase, e.g., rHuPH20, can improve the speed and ease of subcutaneous delivery of injectable biologics and drugs by acting as a permeation enhancer. In certain embodiments, the hyaluronidase comprises ENHANZE™.

In any of the above embodiments, the pharmaceutical formulation may be a unit dosage form.

In an embodiment, the unit dosage form comprises the FcRn antagonist as a dry formulation for dissolution such as a lyophilized powder, freeze-dried powder, or water-free concentrate. In an embodiment, the dry formulation is comprised in a hermetically sealed container such as a vial, an ampoule, or a sachet.

In an embodiment, the unit dosage form comprises the FcRn antagonist as a liquid formulation, (e.g., injection or infusion solution). In an embodiment, the liquid formulation is comprised in a hermetically sealed container such as a vial, a sachet, a pre-filled syringe, a pre-filled autoinjector, or a cartridge for a reusable syringe or applicator.

In an embodiment, the unit dosage per vial may contain 0.5 ml, 1 ml, 2 ml, 3 ml, 4 ml, 5 ml, 6 ml, 7 ml, 8 ml, 9 ml, 10 ml, 15 ml, or 20 ml of an FcRn antagonist ranging from about 500 to about 2500 mg or from about 1000 mg to about 2000 mg. In an embodiment, these preparations can be adjusted to a desired concentration by adding a sterile diluent to each vial.

The formulations disclosed herein include bulk drug compositions useful in the manufacture of pharmaceutical compositions (e.g., compositions that are suitable for administration to a subject or patient) which can be used in the preparation of unit dosage forms. In an embodiment, a composition of the invention is a pharmaceutical composition. Such compositions comprise a prophylactically or therapeutically effective amount of one or more prophylactic or therapeutic agents (e.g., an FcRn antagonist of the invention or other prophylactic or therapeutic agent), and a pharmaceutically acceptable carrier. In an embodiment, the pharmaceutical compositions are formulated to be suitable for subcutaneous administration to a subject.

### Vaccines

As used herein, the term “vaccine” refers to a biological preparation that improves immunity in the context of a particular disease, disorder, or condition.

Vaccines that are useful in the methods and uses provided herein include, but are not limited to, any recombinant vaccine, toxoid vaccine, inactivated vaccine, or live attenuated vaccine. In some embodiments, the vaccine is not a live attenuated vaccine or a live vaccine.

Recombinant vaccines that are useful in the methods and uses provided herein include, but are not limited to an mRNA vaccine, a viral vector vaccine, and a subunit vaccine. Examples of subunit vaccines include, but are not limited to, a polysaccharide vaccine, a conjugate vaccine, and a protein-based vaccine.

In some embodiments, the vaccine is a T-cell dependent vaccine. Examples of T-cell dependent vaccines include, but are not limited to, a COVID-19 vaccine. Examples of COVID-19 vaccines are provided below.

In some embodiments, the vaccine is a seasonal vaccine. In some embodiments, the vaccine is not a seasonal vaccine.

In some embodiments, the vaccine is an adenovirus vaccine, an anthrax vaccine, a cholera vaccine, a COVID-19 vaccine, a cytomegalovirus (CMV) vaccine, a dengue vaccine, a diphtheria vaccine, an Ebola vaccine, a hepatitis A vaccine, a hepatitis B vaccine, a hepatitis C vaccine, a Haemophilus influenzae type B (Hib) vaccine, a human immunodeficiency virus (HIV) vaccine, a human papillomavirus (HPV) vaccine, a seasonal influenza vaccine, a Japanese encephalitis vaccine, a malaria vaccine, a measles vaccine, a meningococcal vaccine, a mumps vaccine, a pertussis vaccine, a pneumococcal vaccine, a polio vaccine, a rabies vaccine, a respiratory syncytial virus (RSV) vaccine, a rotavirus vaccine, a rubella vaccine, a shingles vaccine, a smallpox vaccine, a tetanus vaccine, a tuberculosis vaccine, a typhoid vaccine, a varicella vaccine, a yellow fever vaccine, or any combination thereof. In some embodiments, the vaccine is a COVID-19 vaccine, an influenza vaccine, or a pneumococcal vaccine.

In some embodiments, the vaccine is a COVID-19 vaccine. Any suitable COVID-19 vaccine can be used. In some embodiments, the COVID-19 vaccine comprises one or more SARS-CoV-2 antigens selected from the group consisting of spike protein S2, nucleocapsid, spike protein S1, full-length spike protein, and spike protein receptor-binding domain. Examples of COVID-19 vaccines that may be used in the methods and uses provided herein include, but are not limited to, tozinameran (BNT162b2; Comirnaty; Pfizer), elasomeran (mRNA-1273; Spikevax; Moderna), NVX-CoV2373 (Nuvaxovid/Covovax; Novavax), Ad26.COV2.S (Jcovden; Johnson & Johnson/Janssen), AZD1222 (Vaxzevria; AstraZeneca), CoronaVac (Sinovac), BBV152 (Bharat Biotech), AD5-nCOV (CanSino), MVC-COV1901 (Medigen), BBIBP-CorV (Sinopharm), and Gam-COVID-Vac (Gamaleya).

In some embodiments, the vaccine is an influenza vaccine. Any suitable influenza vaccine can be used. In some embodiments, the influenza vaccine is a seasonal influenza vaccine. Examples of influenza vaccines that may be used in the methods and uses provided herein include, but are not limited to, Audenz, Fluad Quadrivalent, Fluad, Afluria Quadrivalent, Afluria Quadrivalent Southern Hemisphere, Flucelvax Quadrivalent, Flulaval Quadrivalent, Afluria, Afluria Southern Hemisphere, FluLaval, FluMist, Fluarix, Fluvirin, Agriflu, Fluzone, Fluzone High-Dose, Fluzone Intradermal, Flucelvax, Flublok, Flublok Quadrivalent, FluMist Quadrivalent, Fluarix Quadrivalent, and Fluzone Quadrivalent.

In some embodiments, the vaccine is a pneumococcal vaccine. Any suitable pneumococcal vaccine can be used. Examples of pneumococcal vaccines that may be used in the methods and uses provided herein include, but are not limited to, Pneumovax 23, Prevnar 13, VAXNEUVANCE™, and Prevnar 20.

In certain embodiments, the vaccines provided herein are in admixture with one or more adjuvants. Many different adjuvants can be used with the vaccine compositions disclosed herein. The vaccine composition(s) and adjuvant(s) may be mixed together in the same fluid volume, and the vaccine composition(s) can comprise one or more adjuvant(s).

A variety of adjuvants may be employed, including, for example, systemic adjuvants and mucosal adjuvants. A systemic adjuvant is an adjuvant that can be delivered parenterally. Systemic adjuvants include adjuvants that create a depot effect, adjuvants that stimulate the immune system and adjuvants that do both. An adjuvant that creates a depot effect is an adjuvant that causes the antigen to be slowly released in the body, thus prolonging the exposure of immune cells to the antigen. This class of adjuvants includes alum (e.g., aluminum hydroxide, aluminum phosphate); or emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720; Air Liquide, Paris, France); MF59 (a squalene-in-water emulsion stabilized with Span 85 and Tween 80; Chiron Corporation, Emeryville, Calif.); PROVAX (an oil-in-water emulsion containing a stabilizing detergent and a micelle-forming agent; IDEC, Pharmaceuticals Corporation, San Diego, Calif.); and Matrix-M adjuvant.

Other adjuvants stimulate the immune system, for instance, cause an immune cell to produce and secrete cytokines or IgG. This class of adjuvants includes immunostimulatory nucleic acids, such as CpG oligonucleotides; saponins purified from the bark of the Q. saponaria tree, such as QS-21; poly[di(carboxylatophenoxy)phosphazene] (PCPP polymer; Virus Research Institute, USA); RNA mimetics such as polyinosinic:polycytidylic acid (poly I:C) or poly I:C stabilized with poly-lysine (poly-ICLC [Hiltonol®; Oncovir, Inc.]); derivatives of lipopolysaccharides (LPS) such as monophosphoryl lipid A (MPL; Ribi ImmunoChem Research, Inc., Hamilton, Mont.); muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.).

Other systemic adjuvants are adjuvants that create a depot effect and stimulate the immune system. These compounds have both of the above-identified functions of systemic adjuvants. This class of adjuvants includes but is not limited to ISCOMs (Immunostimulating complexes which contain mixed saponins, lipids and form virus-sized particles with pores that can hold antigen; CSL, Melbourne, Australia); AS01 (a liposome based formulation containing MPL and QS-21; GlaxoSmithKline, Belgium); AS02 (an oil-in-water emulsion containing MPL and QS-21; GlaxoSmithKline, Rixensart, Belgium); AS04 (contains alum and MPL; GlaxoSmithKline, Belgium); AS15 (a liposome based formulation containing CpG oligonucleotides, MPL and QS-21; GlaxoSmithKline, Belgium); non-ionic block copolymers that form micelles such as CRL 1005 (these contain a linear chain of hydrophobic polyoxypropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

The mucosal adjuvants useful according to the invention are adjuvants that are capable of inducing a mucosal immune response in a subject when administered to a mucosal surface. Mucosal adjuvants include CpG nucleic acids (e.g., PCT published patent application WO 99/61056, incorporated herein by reference in its entirety), bacterial toxins: e.g., Cholera toxin (CT), CT derivatives including but not limited to CT B subunit (CTB); CTD53 (Val to Asp); CTK97 (Val to Lys); CTK104 (Tyr to Lys); CTD53/K63 (Val to Asp, Ser to Lys); CTH54 (Arg to His); CTN107 (His to Asn); CTE114 (Ser to Glu); CTE112K (Glu to Lys); CTS61F (Ser to Phe); CTS 106 (Pro to Lys); and CTK63 (Ser to Lys), Zonula occludens toxin (zot), Escherichia coli heat-labile enterotoxin, Labile Toxin (LT), LT derivatives including but not limited to LT B subunit (LTB); LT7K (Arg to Lys); LT61F (Ser to Phe); LT112K (Glu to Lys); LT118E (Gly to Glu); LT146E (Arg to Glu); LT192G (Arg to Gly); LTK63 (Ser to Lys); and LTR72 (Ala to Arg), Pertussis toxin, PT including PT-9K/129G; Toxin derivatives; Lipid A derivatives (e.g., monophosphoryl lipid A, MPL); Muramyl Dipeptide (MDP) derivatives; bacterial outer membrane proteins (e.g., outer surface protein A (OspA) lipoprotein of Borrelia burgdorferi, outer membrane protein of Neisseria meningitidis); oil-in-water emulsions (e.g., MF59; aluminum salts (Isaka et al., 1998, 1999)); and Saponins (e.g., QS-21, e.g., QS-21 Stimulon®, Antigenics LLC, Lexington, Mass.), ISCOMs, MF59 (a squalene-in-water emulsion stabilized with Span 85 and Tween 80; Chiron Corporation, Emeryville, Calif.); the Seppic ISA series of Montanide adjuvants (e.g., Montanide ISA 720; Air Liquide, Paris, France); PROVAX (an oil-in-water emulsion containing a stabilizing detergent and a micelle-forming agent; IDEC Pharmaceuticals Corporation, San Diego, Calif.); Syntext Adjuvant Formulation (SAF; Syntex Chemicals, Inc., Boulder, Colo.); poly[di(carboxylatophenoxy)phosphazene] (PCPP polymer; Virus Research Institute, USA) and Leishmania elongation factor (Corixa Corporation, Seattle, Wash.).

Adjuvant(s) that can be added to the vaccine compositions disclosed herein include, for example, saponins, and immunostimulatory nucleic acids.

Pharmaceutical compositions for vaccines can be optionally prepared as lyophilized product, which may then be formulated for oral administration or reconstituted to a liquid form for parenteral administration.

Pharmaceutical compositions for vaccines can additionally be formulated to contain other agents as pharmaceutically acceptable carriers or excipients, including bulking agents, stabilizing agents, buffering agents, sodium chloride, calcium salts, surfactants, antioxidants, chelating agents, other excipients, and combinations thereof.

Bulking agents are preferred in the preparation of lyophilized formulations for vaccines. Such bulking agents form the crystalline portion of the lyophilized product and may be selected from the group consisting of mannitol, glycine, alanine, and hydroxyethyl starch (HES).

Stabilizing agents may be selected from the group consisting of sucrose, trehalose, raffinose, and arginine. These agents are preferably present in amounts between 1-4%.

Sodium chloride can be included in the present vaccine formulations preferably in an amount of 100-300 mM, or if used without the aforementioned bulking agents, can be included in the formulations in an amount of between 300-500 mM NaCl. Calcium salts include calcium chloride, calcium gluconate, calcium glubionate, or calcium gluceptate.

Buffering agents can be any physiologically acceptable chemical entity or combination of chemical entities which have a capacity to act as buffers, including but not limited to histidine, potassium phosphate, TRIS [tris-(hydroxymethyl)-aminomethane], BIS-Tris Propane (1,3-bis-[tris-(hydroxymethyl)methylamino]-propane), PIPES [piperazine-N,N′-bis-(2-ethanesulfonic acid)], MOPS [3-(N-morpholino)ethanesulfonic acid], HEPES (N-2-hydroxyethyl-piperazine-N′-2-ethanesulfonic acid), MES [2-(N-morpholino)ethanesulfonic acid], and ACES (N-2-acetamido-2-aminoethanesulfonic acid). Typically, the buffering agent is included in the vaccine formulation in a concentration of 10-50 mM. Specific examples of base buffers include (i) PBS; (ii) 10 mM KPO.sub.4, 150 mM NaCl; (iii) 10 mM HEPES, 150 mM NaCl; (iv) 10 mM imidazole, 150 mM NaCl; and (v) 20 mM sodium citrate. Excipients that can be used include (i) glycerol (10%, 20%); (ii) Tween 50 (0.05%, 0.005%); (iii) 9% sucrose; (iv) 20% sorbitol; (v) 10 mM lysine; or (vi) 0.01 mM dextran sulfate.

Surfactants, if present, are preferably in a concentration of 0.1% or less, and may be chosen from the group including but not limited to polysorbate 20, polysorbate 80, pluronic polyols, and BRIJ 35 (polyoxyethylene 23 laurel ether). Antioxidants, if used, must be compatible for use with a pharmaceutical preparation, and are preferably water soluble. Suitable antioxidants include homocysteine, glutathione, lipoic acid, 6-hydroxy-2,5,7,8-tetramethylchroman-2-carboxylic acid (Trolox), methionine, sodium thiosulfate, platinum, glycine-glycine-histidine (tripeptide), and butylatedhydroxytoluene (BHT). Chelating agents should preferably bind metals such as copper and iron with greater affinity than calcium, if a calcium salt is being used in the composition. A preferred chelator is deferoxamine.

The vaccines disclosed herein may be administered using any desired route of administration. Many methods may be used to introduce the vaccines described herein, including but not limited to, oral, intradermal, intramuscular, intraperitoneal, intravenous, subcutaneous, mucosal, intranasal, intra-tumoral, and intra-lymph node routes. Non-mucosal routes of administration include, but are not limited to, intradermal and topical administration. Mucosal routes of administration include, but are not limited to, oral, rectal, and nasal administration. Advantages of intradermal administration include use of lower doses and rapid absorption, respectively. Advantages of subcutaneous or intramuscular administration include suitability for some insoluble suspensions and oily suspensions, respectively.

Solubility and the site of the administration are factors which should be considered when choosing the route of administration of the vaccines. The mode of administration can be varied between multiple routes of administration, including those listed above.

If the vaccines are water-soluble, then they may be formulated in an appropriate buffer, for example, phosphate buffered saline or other physiologically compatible solutions, preferably sterile. Alternatively, if a vaccine has poor solubility in aqueous solvents, then it may be formulated with a non-ionic surfactant such as Tween, or polyethylene glycol. Thus, the compositions may be formulated for administration by inhalation or insufflation (either through the mouth or the nose) or oral, buccal, parenteral, or rectal administration.

The vaccines may be formulated for parenteral administration by injection, e.g., by bolus injection or continuous infusion. Formulations for injection may be presented in unit dosage form, e.g., in ampoules or in multi-dose containers, with an added preservative. The preparation may take such forms as suspensions, solutions, or emulsions in oily or aqueous vehicles, and may contain formulatory agents such as suspending, stabilizing, and/or dispersing agents. Alternatively, the vaccine may be in powder form for constitution with a suitable vehicle, e.g., sterile pyrogen-free water, before use.

The vaccine may also be formulated in a rectal preparation such as a suppository or retention enema, e.g., containing conventional suppository bases such as cocoa butter or other glycerides.

In addition, the vaccine may also be formulated as a depot preparation. Such long-acting formulations may be administered by implantation (for example, subcutaneously or intramuscularly) or by intramuscular injection. Thus, for example, the preparation may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

### Methods

The instant disclosure provides methods of reducing serum IgG in a subject comprising administering to the subject an effective amount of an FcRn antagonist according to the disclosure or a pharmaceutical composition comprising the same within about 2 months or about 1 month of administration of a vaccine to the subject. In some embodiments, the FcRn antagonist or pharmaceutical composition comprising the same is administered to the subject within about 8 weeks, about 7 weeks, about 6 weeks, about 5 weeks, about 4 weeks, about 3 weeks, about 2 weeks, or about 1 week of administration of a vaccine to the subject. In some embodiments, the FcRn antagonist or pharmaceutical composition comprising the same is administered to the subject within 2 months or 1 month of administration of a vaccine to the subject. In some embodiments, the FcRn antagonist or pharmaceutical composition comprising the same is administered to the subject within 8 weeks, 7 weeks, 6 weeks, 5 weeks, 4 weeks, 3 weeks, 2 weeks, or 1 week of administration of a vaccine to the subject.

In some embodiments, the FcRn antagonist or pharmaceutical composition comprising the same is administered to the subject after administration of a vaccine to the subject. In some embodiments, the FcRn antagonist or pharmaceutical composition comprising the same administered to the subject after administration of the vaccine to the subject is an initial dose of the FcRn antagonist. As used herein, an initial dose of an FcRn antagonist is a first administration of any FcRn antagonist, a first administration of a particular FcRn antagonist, a first administration of an FcRn antagonist in a treatment regimen, or a first administration of an FcRn antagonist in a dosing cycle.

In some embodiments, the FcRn antagonist or pharmaceutical composition comprising the same is administered to the subject ≤4 weeks, ≤3 weeks, ≤2 weeks, or ≤1 week after administration of the vaccine to the subject. In some embodiments, the FcRn antagonist or pharmaceutical composition comprising the same is administered to the subject ≤28 days, ≤27 days, ≤26 days, ≤25 days, ≤24 days, ≤23 days, ≤22 days, ≤21 days, ≤20 days, ≤19 days, ≤18 days, ≤17 days, ≤16 days, 15 days, ≤14 days, ≤13 days, ≤12 days, 11 days, ≤10 days, ≤9 days, ≤8 days, ≤7 days, ≤6 days, ≤5 days, ≤4 days, ≤3 days, ≤2 days, or ≤1 day after administration of the vaccine to the subject. In some embodiments, the FcRn antagonist or pharmaceutical composition comprising the same is administered to the subject ≤24 hours after administration of the vaccine to the subject.

In some embodiments, FcRn antagonist or pharmaceutical composition comprising the same is administered to the subject about 4 weeks, about 3 weeks, about 2 weeks, or about 1 week after administration of the vaccine to the subject. In some embodiments, the FcRn antagonist or pharmaceutical composition comprising the same is administered to the subject about 28 days, about 27 days, about 26 days, about 25 days, about 24 days, about 23 days, about 22 days, about 21 days, about 20 days, about 19 days, about 18 days, about 17 days, about 16 days, about 15 days, about 14 days, about 13 days, about 12 days, about 11 days, about 10 days, about 9 days, about 8 days, about 7 days, about 6 days, about 5 days, about 4 days, about 3 days, about 2 days, or about 1 day after administration of the vaccine to the subject. In some embodiments, the FcRn antagonist or pharmaceutical composition comprising the same is administered to the subject 4 weeks, 3 weeks, 2 weeks, or 1 week after administration of the vaccine to the subject. In some embodiments, the FcRn antagonist or pharmaceutical composition comprising the same is administered to the subject 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, or 1 day after administration of the vaccine to the subject. In some embodiments, the FcRn antagonist or pharmaceutical composition comprising the same is administered to the subject after administration of the vaccine to the subject on the same day.

In some embodiments, the FcRn antagonist or pharmaceutical composition comprising the same is administered to the subject before administration of a vaccine to the subject. In some embodiments, the FcRn antagonist or pharmaceutical composition comprising the same is administered to the subject ≤2 months or ≤1 month before administration of the vaccine to the subject. In some embodiments, the FcRn antagonist or pharmaceutical composition comprising the same is administered to the subject ≤8 weeks, ≤7 weeks, ≤6 weeks, ≤5 weeks, ≤4 weeks, ≤3 weeks, ≤2 weeks, or ≤1 week before administration of the vaccine to the subject. In some embodiments, the FcRn antagonist or pharmaceutical composition comprising the same is administered to the subject ≤60 days, ≤59 days, ≤58 days, ≤57 days, ≤56 days, ≤55 days, ≤54 days, ≤53 days, ≤52 days, ≤51 days, ≤50 days, ≤49 days, ≤48 days, ≤47 days, ≤46 days, ≤45 days, ≤44 days, ≤43 days, ≤42 days, ≤41 days, ≤40 days, ≤39 days, ≤38 days, ≤37 days, ≤36 days, ≤35 days, ≤34 days, ≤33 days, ≤32 days, ≤31 days, ≤30 days, ≤29 days, ≤28 days, ≤27 days, ≤26 days, ≤25 days, ≤24 days, ≤23 days, ≤22 days, ≤21 days, ≤20 days, ≤19 days, ≤18 days, ≤17 days, ≤16 days, ≤15 days, ≤14 days, ≤13 days, ≤12 days, ≤11 days, ≤10 days, ≤9 days, ≤8 days, ≤7 days, ≤6 days, ≤5 days, ≤4 days, ≤3 days, ≤2 days, or ≤1 day before administration of the vaccine to the subject. In some embodiments, the FcRn antagonist or pharmaceutical composition comprising the same is administered to the subject ≤24 hours before administration of the vaccine to the subject.

In some embodiments, the FcRn antagonist or pharmaceutical composition comprising the same is administered to the subject about 2 months or about 1 month before administration of the vaccine to the subject. In some embodiments, the FcRn antagonist or pharmaceutical composition comprising the same is administered to the subject about 8 weeks, about 7 weeks, about 6 weeks, about 5 weeks, about 4 weeks, about 3 weeks, about 2 weeks, or about 1 week before administration of the vaccine to the subject. In some embodiments, the FcRn antagonist or pharmaceutical composition comprising the same is administered to the subject about 60 days, about 59 days, about 58 days, about 57 days, about 56 days, about 55 days, about 54 days, about 53 days, about 52 days, about 51 days, about 50 days, about 49 days, about 48 days, about 47 days, about 46 days, about 45 days, about 44 days, about 43 days, about 42 days, about 41 days, about 40 days, about 39 days, about 38 days, about 37 days, about 36 days, about 35 days, about 34 days, about 33 days, about 32 days, about 31 days, about 30 days, about 29 days, about 28 days, about 27 days, about 26 days, about 25 days, about 24 days, about 23 days, about 22 days, about 21 days, about 20 days, about 19 days, about 18 days, about 17 days, about 16 days, about 15 days, about 14 days, about 13 days, about 12 days, about 11 days, about 10 days, about 9 days, about 8 days, about 7 days, about 6 days, about 5 days, about 4 days, about 3 days, about 2 days, or about 1 day before administration of the vaccine to the subject.

In some embodiments, the FcRn antagonist or pharmaceutical composition comprising the same is administered to the subject 2 months or 1 month before administration of the vaccine to the subject. In some embodiments, the FcRn antagonist or pharmaceutical composition comprising the same is administered to the subject 8 weeks, 7 weeks, 6 weeks, 5 weeks, 4 weeks, 3 weeks, 2 weeks, or 1 week before administration of the vaccine to the subject. In some embodiments, the FcRn antagonist or pharmaceutical composition comprising the same is administered to the subject 60 days, 59 days, 58 days, 57 days, 56 days, 55 days, 54 days, 53 days, 52 days, 51 days, 50 days, 49 days, 48 days, 47 days, 46 days, 45 days, 44 days, 43 days, 42 days, 41 days, 40 days, 39 days, 38 days, 37 days, 36 days, 35 days, 34 days, 33 days, 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, or 1 day before administration of the vaccine to the subject. In some embodiments, the FcRn antagonist or pharmaceutical composition comprising the same is administered to the subject before administration of the vaccine to the subject on the same day.

In some embodiments, the FcRn antagonist or pharmaceutical composition comprising the same is administered to the subject on the same day as administration of the vaccine to the subject.

In some embodiments, multiple doses of the FcRn antagonist are administered to the subject. Thus, in some embodiments the administration of the FcRn antagonist to the subject comprises administration of an initial dose of the FcRn antagonist and one or more subsequent doses of the FcRn antagonist. In some embodiments, a subsequent dose of the FcRn antagonist is administered to the subject ≤4 weeks, ≤3 weeks, ≤2 weeks, or ≤1 week after administration of the vaccine to the subject. In some embodiments, a subsequent dose of the FcRn antagonist is administered to the subject ≤7 days, ≤6 days, ≤5 days, ≤4 days, ≤3 days, ≤2 days, or ≤1 day after administration of the vaccine to the subject. In some embodiments, a subsequent dose of the FcRn antagonist is administered to the subject ≤24 hours after administration of the vaccine to the subject. In some embodiments, a subsequent dose of the FcRn antagonist is administered to the subject on the same day as administration of the vaccine to the subject.

In an embodiment, the level of serum IgG is decreased in the subject following administration of the FcRn antagonist compared to a baseline level of serum IgG. In an embodiment, a total serum IgG reduction of about 60% compared to baseline serum IgG level is obtained. In an embodiment, a total serum IgG reduction of about 65%, about 70%, about 75%, or about 80% compared to baseline serum IgG level is obtained. In an embodiment, a total serum IgG reduction of about 65% compared to baseline serum IgG level is obtained. In an embodiment, a total serum IgG reduction of about 70% compared to baseline serum IgG level is obtained. In an embodiment, a total serum IgG reduction of about 75% compared to baseline serum IgG level is obtained. In an embodiment, a total serum IgG reduction of about 80% compared to baseline serum IgG level is obtained.

In an embodiment, the level of FcRn is not decreased in the subject following administration of the FcRn antagonist molecule compared to a baseline level of FcRn. In an embodiment, an FcRn reduction of less than about 1%, 2%, 3%, 4%, or 5% compared to baseline FcRn level is observed. In an embodiment, an FcRn reduction of less than about 10% compared to baseline FcRn level is observed.

The disclosure also provides methods for treating an antibody-mediated disorder (e.g., an autoantibody-mediated disorder) in a subject comprising administering to the subject a therapeutically effective amount of an FcRn antagonist according to the disclosure or a pharmaceutical composition comprising the same.

In some embodiments, the antibody-mediated disorder is an autoimmune disease. In some embodiments, the autoimmune disease is selected from the group consisting of allogenic islet graft rejection, alopecia areata, ankylosing spondylitis, antiphospholipid syndrome, autoimmune Addison's disease, Alzheimer's disease, antineutrophil cytoplasmic autoantibodies (ANCA), autoimmune diseases of the adrenal gland, autoimmune hemolytic anemia, autoimmune hepatitis, autoimmune myocarditis, autoimmune neutropenia, autoimmune oophoritis and orchitis, immune thrombocytopenia (ITP or idiopathic thrombocytopenic purpura, idiopathic thrombocytopenia purpura, immune mediated thrombocytopenia, or primary immune thrombocytopenia), autoimmune urticaria, Behcet's disease, bullous pemphigoid (BP), cardiomyopathy, Castleman disease, celiac sprue-dermatitis, chronic fatigue immune disfunction syndrome, chronic inflammatory demyelinating polyneuropathy (CIDP), Churg-Strauss syndrome, cicatricial pemphigoid, CREST syndrome, cold agglutinin disease, Crohn's disease, dilated cardiomyopathy, discoid lupus, epidermolysis bullosa acquisita, essential mixed cryoglobulinemia, factor VIII deficiency, fibromyalgia-fibromyositis, glomerulonephritis, Goodpasture syndrome, Graves' disease, Guillain-Barre syndrome, graft-versus-host disease (GVHD), Hashimoto's thyroiditis, hemophilia A, idiopathic inflammatory myopathies (IIMs), idiopathic membranous neuropathy, idiopathic pulmonary fibrosis, IgA neuropathy, IgM polyneuropathies, immune-mediated necrotizing myopathy (IMNM), juvenile arthritis, Kawasaki's disease, lichen planus, lichen sclerosus, lupus erythematosus, lupus nephritis, Ménière's disease, mixed connective tissue disease, mucous membrane pemphigoid, multiple sclerosis, Type 1 diabetes mellitus, multifocal motor neuropathy (MMN), myasthenia gravis (MG), generalized myasthenia gravis (gMG), myositis, paraneoplastic bullous pemphigoid, pemphigoid gestationis, Pemphigus vulgaris (PV), Pemphigus foliaceus (PF), pernicious anemia, polyarteritis nodosa, polychondritis, polyglandular syndromes, polymyalgia rheumatica, polymyositis, dermatomyositis (DM), necrotizing autoimmune myopathy (NAM), AntiSynthetase Syndrome (ASyS), primary agammaglobulinemia, primary biliary cirrhosis, psoriasis, psoriatic arthritis, relapsing polychondritis, Raynaud's phenomenon, Reiter's syndrome, rheumatoid arthritis, sarcoidosis, scleroderma, Sjögren's syndrome, solid organ transplant rejection, stiff-person syndrome, systemic lupus erythematosus, Takayasu's arteritis, toxic epidermal necrolysis (TEN), Stevens-Johnson syndrome (SJS), temporal arteritis/giant cell arteritis, thrombotic thrombocytopenia purpura, ulcerative colitis, uveitis, dermatitis herpetiformis vasculitis, anti-neutrophil cytoplasmic antibody-associated vasculitides, vitiligo, and Wegener's granulomatosis.

In an embodiment, at least one of the IgG subtypes is reduced in a subject following administration of the FcRn antagonist. In an embodiment, at least one of the IgG subtypes is reduced in serum of a subject following administration of the FcRn antagonist. In an embodiment, IgG1 is reduced. In an embodiment, IgG2 is reduced. In an embodiment, IgG3 is reduced. In an embodiment, IgG4 is reduced. In some embodiments, total serum IgG is reduced by at least 10%, at least 20%, at least 30%, at least 35%, at least 40%, at least 45%, at least 50%, at least 55%, at least 60%, at least 65%, or at least 70% in a subject following a single administration of the FcRn antagonist.

In some embodiments, clearance of total serum IgG is increased in a subject following administration of the FcRn antagonist. In some embodiments, clearance of total serum IgG in a subject following a single therapeutic administration of the FcRn antagonist is comparable to the clearance of total serum IgG in a subject following a single therapeutic administration of efgartigimod. In some embodiments, clearance of total serum IgG in a subject following a single therapeutic administration of the FcRn antagonist is similar or the same as the clearance of total serum IgG in a subject following a single therapeutic administration of efgartigimod.

In some embodiments, clearance of total serum IgG in a subject following a single administration of the FcRn antagonist is comparable to the clearance of total serum IgG in a subject following a single administration of an equivalent amount of efgartigimod. In some embodiments, clearance of total serum IgG in a subject following a single administration of the FcRn antagonist is similar or the same as the clearance of total serum IgG in a subject following a single administration of an equivalent amount of efgartigimod.

In an embodiment, the FcRn antagonist is administered to the subject simultaneously or sequentially with an additional therapeutic agent. In an embodiment, the additional therapeutic agent is an anti-inflammatory agent. In an embodiment, the additional therapeutic agent is a corticosteroid. In an embodiment, the additional therapeutic agent is rituximab, daclizumab, basiliximab, muromonab-CD3, infliximab, adalimumab, omalizumab, efalizumab, natalizumab, tocilizumab, eculizumab, golimumab, canakinumab, ustekinumab, or belimumab. In an embodiment, the additional therapeutic agent is a leucocyte depleting agent.

In an embodiment, the additional therapeutic agent is a B-cell depleting agent. In an embodiment, the B-cell depleting agent is an antibody. In an embodiment, the B-cell depleting antibody is an antibody that specifically binds to CD10, CD19, CD20, CD21, CD22, CD23, CD24, CD37, CD53, CD70, CD72, CD74, CD75, CD77, CD79a, CD79b, CD80, CD81, CD82, CD83, CD84, CD85, or CD86.

In some embodiments, the FcRn antagonist is administered at a fixed dose of about 20 mg to about 20,000 mg. In some embodiments, the FcRn antagonist is administered at a fixed dose of about 200 mg to about 20,000 mg. In some embodiments, the FcRn antagonist is administered at a fixed dose of about 300 mg to about 6000 mg. In some embodiments, the FcRn antagonist is administered at a fixed dose of about 750 mg to about 3000 mg. In some embodiments, the FcRn antagonist is administered at a fixed dose of about 1000 mg to about 2500 mg. In some embodiments, the FcRn antagonist is administered at a fixed dose of about 1000 mg to about 2000 mg. In some embodiments, the FcRn antagonist is efgartigimod.

In some embodiments, the FcRn antagonist is administered at a fixed dose of 20 mg to 20,000 mg. In some embodiments, the FcRn antagonist is administered at a fixed dose of 200 mg to 20,000 mg. In some embodiments, the FcRn antagonist is administered at a fixed dose of 300 mg to 6000 mg. In some embodiments, the FcRn antagonist is administered at a fixed dose of 750 mg to 3000 mg. In some embodiments, the FcRn antagonist is administered at a fixed dose of 1000 mg to 2500 mg. In some embodiments, the FcRn antagonist is administered at a fixed dose of 1000 mg to 2000 mg. In some embodiments, the FcRn antagonist is efgartigimod.

In some embodiments, the FcRn antagonist is administered at a fixed dose of about 20 mg, about 50 mg, about 100 mg, about 200 mg, about 250 mg, about 300 mg, about 500 mg, about 750 mg, about 1000 mg, about 1500 mg, about 2000 mg, about 2500 mg, about 3000 mg, about 4000 mg, about 5000 mg, about 6000 mg, about 7000 mg, about 8000 mg, about 9000 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, or about 20,000 mg. In some embodiments, the FcRn antagonist is efgartigimod.

In some embodiments, the FcRn antagonist is administered at a fixed dose of 20 mg, 50 mg, 100 mg, 200 mg, 250 mg, 300 mg, 500 mg, 750 mg, 1000 mg, 1500 mg, 2000 mg, 2500 mg, 3000 mg, 4000 mg, 5000 mg, 6000 mg, 7000 mg, 8000 mg, 9000 mg, 10,000 mg, 11,000 mg, 12,000 mg, 13,000 mg, 14,000 mg, 15,000 mg, 16,000 mg, 17,000 mg, 18,000 mg, 19,000 mg, or 20,000 mg. In some embodiments, the FcRn antagonist is efgartigimod.

In some embodiments, the FcRn antagonist is administered at a dose of about 0.2 mg/kg to about 200 mg/kg. In some embodiments, the FcRn antagonist is administered at a dose of about 2 mg/kg to about 200 mg/kg. In some embodiments, the FcRn antagonist is administered at a dose of about 2 mg/kg to about 120 mg/kg. In some embodiments, the FcRn antagonist is administered at a dose of about 3 mg/kg to about 60 mg/kg. In some embodiments, the FcRn antagonist is administered at a dose of about 10 mg/kg to about 25 mg/kg. In some embodiments, the FcRn antagonist is efgartigimod.

In some embodiments, the FcRn antagonist is administered at a dose of 0.2 mg/kg to 200 mg/kg. In some embodiments, the FcRn antagonist is administered at a dose of about 2 mg/kg to about 200 mg/kg. In some embodiments, the FcRn antagonist is administered at a dose of 2 mg/kg to 120 mg/kg. In some embodiments, the FcRn antagonist is administered at a dose of 3 mg/kg to 60 mg/kg. In some embodiments, the FcRn antagonist is administered at a dose of 10 mg/kg to 25 mg/kg. In some embodiments, the FcRn antagonist is efgartigimod.

In some embodiments, the FcRn antagonist is administered at a dose of about 0.2 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12.5 mg/kg, about 15 mg/kg, about 17.5 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, or about 200 mg/kg. In some embodiments, the FcRn antagonist is efgartigimod.

In some embodiments, the FcRn antagonist is administered at a dose of 0.2 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, or 200 mg/kg. In some embodiments, the FcRn antagonist is efgartigimod.

In some embodiments, the FcRn antagonist is administered intravenously. In some embodiments, the FcRn antagonist is administered intravenously once weekly, once every 2 weeks, once every 3 weeks, once every 4 weeks, once monthly, or once every 6 weeks. In some embodiments, the FcRn antagonist is efgartigimod.

In some embodiments, the FcRn antagonist is administered intravenously once weekly or once every 2 weeks at a dose of about 0.2 mg/kg to about 200 mg/kg. In some embodiments, the FcRn antagonist is administered intravenously once weekly or once every 2 weeks at a dose of about 2 mg/kg to about 200 mg/kg. In some embodiments, the FcRn antagonist is administered intravenously once weekly or once every 2 weeks at a dose of about 2 mg/kg to about 120 mg/kg. In some embodiments, the FcRn antagonist is administered intravenously once weekly or once every 2 weeks at a dose of about 3 mg/kg to about 60 mg/kg. In some embodiments, the FcRn antagonist is administered intravenously once weekly or once every 2 weeks at a dose of about 10 mg/kg to about 25 mg/kg. In some embodiments, the FcRn antagonist is efgartigimod.

In some embodiments, the FcRn antagonist is administered intravenously once weekly or once every 2 weeks at a dose of 0.2 mg/kg to 200 mg/kg. In some embodiments, the FcRn antagonist is administered intravenously once weekly or once every 2 weeks at a dose of 2 mg/kg to 200 mg/kg. In some embodiments, the FcRn antagonist is administered intravenously once weekly or once every 2 weeks at a dose of 2 mg/kg to 120 mg/kg. In some embodiments, the FcRn antagonist is administered intravenously once weekly or once every 2 weeks at a dose of 3 mg/kg to 60 mg/kg. In some embodiments, the FcRn antagonist is administered intravenously once weekly or once every 2 weeks at a dose of 10 mg/kg to 25 mg/kg. In some embodiments, the FcRn antagonist is efgartigimod.

In some embodiments, the FcRn antagonist is administered intravenously once weekly or once every 2 weeks at a dose of about 0.2 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12.5 mg/kg, about 15 mg/kg, about 17.5 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, or about 200 mg/kg. In some embodiments, the FcRn antagonist is efgartigimod.

In some embodiments, the FcRn antagonist is administered intravenously once weekly or once every 2 weeks at a dose of 0.2 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, or 200 mg/kg. In some embodiments, the FcRn antagonist is efgartigimod.

In some embodiments, the FcRn antagonist is administered intravenously once weekly or once every 2 weeks at a dose of about 10 mg/kg to about 30 mg/kg. In some embodiments, the FcRn antagonist is administered intravenously once weekly or once every 2 weeks at a dose of about 10 mg/kg to about 25 mg/kg. In some embodiments, the FcRn antagonist is administered intravenously once weekly or once every 2 weeks at a dose of about 10 mg/kg. In some embodiments, the FcRn antagonist is administered intravenously once weekly or once every 2 weeks at a dose of about 15 mg/kg. In some embodiments, the FcRn antagonist is administered intravenously once weekly or once every 2 weeks at a dose of about 20 mg/kg. In some embodiments, the FcRn antagonist is administered intravenously once weekly or once every 2 weeks at a dose of about 25 mg/kg. In some embodiments, the FcRn antagonist is administered intravenously once weekly or once every 2 weeks at a dose of about 30 mg/kg. In some embodiments, the FcRn antagonist is administered intravenously once weekly or once every 2 weeks at a dose of 10 mg/kg to 30 mg/kg. In some embodiments, the FcRn antagonist is administered intravenously once weekly or once every 2 weeks at a dose of 10 mg/kg to 25 mg/kg. In some embodiments, the FcRn antagonist is administered intravenously once weekly or once every 2 weeks at a dose of 10 mg/kg. In some embodiments, the FcRn antagonist is administered intravenously once weekly or once every 2 weeks at a dose of 15 mg/kg. In some embodiments, the FcRn antagonist is administered intravenously once weekly or once every 2 weeks at a dose of 20 mg/kg. In some embodiments, the FcRn antagonist is administered intravenously once weekly or once every 2 weeks at a dose of 25 mg/kg. In some embodiments, the FcRn antagonist is administered intravenously once weekly or once every 2 weeks at a dose of 30 mg/kg. In some embodiments, the FcRn antagonist is efgartigimod.

In some embodiments, the FcRn antagonist is administered subcutaneously. In some embodiments, the FcRn antagonist is administered subcutaneously once weekly, once every 2 weeks, once every 3 weeks, once every 4 weeks, once monthly, or once every 6 weeks. In some embodiments, the FcRn antagonist is efgartigimod.

In some embodiments, FcRn antagonist is administered subcutaneously at a fixed dose of about 20 mg to about 20,000 mg. In some embodiments, FcRn antagonist is administered subcutaneously at a fixed dose of about 100 mg to about 10,000 mg once weekly, once every 2 weeks, once every 3 weeks, once every 4 weeks, once monthly, or once every 6 weeks. In some embodiments, the FcRn antagonist is administered subcutaneously at a fixed dose of 750 mg to 3000 mg once weekly, once every 2 weeks, once every 3 weeks, once every 4 weeks, once monthly, or once every 6 weeks. In some embodiments, the FcRn antagonist is administered subcutaneously at a fixed dose of 1000 mg to 2000 mg once weekly, once every 2 weeks, once every 3 weeks, once every 4 weeks, once monthly, or once every 6 weeks. In some embodiments, the FcRn antagonist is efgartigimod.

In some embodiments, the FcRn antagonist is administered subcutaneously at a fixed dose of about 20 mg, about 50 mg, about 100 mg, about 250 mg, about 500 mg, about 750 mg, about 1000 mg, about 1500 mg, about 2000 mg, about 3000 mg, about 4000 mg, about 5000 mg, about 6000 mg, about 7000 mg, about 8000 mg, about 9000 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, or about 20,000 mg once weekly, once every 2 weeks, once every 3 weeks, once every 4 weeks, once monthly, or once every 6 weeks. In some embodiments, the FcRn antagonist is efgartigimod.

In some embodiments, the FcRn antagonist is administered subcutaneously at a fixed dose of 20 mg, 50 mg, 100 mg, 250 mg, 500 mg, 750 mg, 1000 mg, 1500 mg, 2000 mg, 3000 mg, 4000 mg, 5000 mg, 6000 mg, 7000 mg, 8000 mg, 9000 mg, 10,000 mg, 11,000 mg, 12,000 mg, 13,000 mg, 14,000 mg, 15,000 mg, 16,000 mg, 17,000 mg, 18,000 mg, 19,000 mg, or 20,000 mg once weekly, once every 2 weeks, once every 3 weeks, once every 4 weeks, once monthly, or once every 6 weeks. In some embodiments, the FcRn antagonist is administered subcutaneously at a fixed dose of 1000 mg or 2000 mg once weekly, once every 2 weeks, once every 3 weeks, once every 4 weeks, once monthly, or once every 6 weeks. In some embodiments, the FcRn antagonist is efgartigimod.

In some embodiments, the FcRn antagonist is administered subcutaneously once weekly or once every 2 weeks at a fixed dose of about 750 mg to about 3000 mg. In some embodiments, the FcRn antagonist is administered subcutaneously once weekly or once every 2 weeks at a fixed dose of about 1000 mg to about 2000 mg. In some embodiments, the FcRn antagonist is administered subcutaneously at a fixed dose of about 1000 mg or about 2000 mg once weekly or once every 2 weeks. In some embodiments, the FcRn antagonist is efgartigimod.

In some embodiments, the FcRn antagonist is administered subcutaneously once weekly or once every 2 weeks at a fixed dose of 750 mg to 3000 mg. In some embodiments, the FcRn antagonist is administered subcutaneously at a fixed dose of 750 mg to 3000 mg once weekly. In some embodiments, the FcRn antagonist is administered subcutaneously at a fixed dose of 750 mg to 3000 mg once every 2 weeks. In some embodiments, the FcRn antagonist is administered subcutaneously at a fixed dose of 750 mg to 3000 mg once every 3 weeks. In some embodiments, the FcRn antagonist is administered subcutaneously at a fixed dose of 750 mg to 3000 mg once monthly. In some embodiments, the FcRn antagonist is administered subcutaneously once weekly or once every 2 weeks at a fixed dose of 1000 mg to 2000 mg. In some embodiments, the FcRn antagonist is administered subcutaneously at a fixed dose of 1000 mg or 2000 mg once weekly or once every 2 weeks. In some embodiments, the FcRn antagonist is efgartigimod.

In some embodiments, the FcRn antagonist is first administered subcutaneously at a fixed dose of about 1000 mg twice on the same day. In some embodiments, the FcRn antagonist is first administered subcutaneously at a fixed dose of 1000 mg twice on the same day. In some embodiments, the FcRn antagonist is efgartigimod.

In some embodiments, the FcRn antagonist is administered subcutaneously once weekly at a fixed dose of about 750 mg to about 1750 mg. In some embodiments, the FcRn antagonist is administered subcutaneously once weekly at a fixed dose of about 800 mg to about 1200 mg. In some embodiments, the FcRn antagonist is administered subcutaneously once weekly at a fixed dose of about 750 mg. In some embodiments, the FcRn antagonist is administered subcutaneously once weekly at a fixed dose of about 800 mg. In some embodiments, the FcRn antagonist is administered subcutaneously once weekly at a fixed dose of about 1000 mg. In some embodiments, the FcRn antagonist is administered subcutaneously once weekly at a fixed dose of about 1200 mg. In some embodiments, the FcRn antagonist is administered subcutaneously once weekly at a fixed dose of about 1250 mg. In some embodiments, the FcRn antagonist is administered subcutaneously once weekly at a fixed dose of about 1500 mg. In some embodiments, the FcRn antagonist is administered subcutaneously once weekly at a fixed dose of about 1750 mg. In some embodiments, the FcRn antagonist is efgartigimod.

In some embodiments, the FcRn antagonist is administered subcutaneously once weekly at a fixed dose of 750 mg to 1750 mg. In some embodiments, the FcRn antagonist is administered subcutaneously once weekly at a fixed dose of 800 mg to 1200 mg. In some embodiments, the FcRn antagonist is administered subcutaneously once weekly at a fixed dose of 750 mg. In some embodiments, the FcRn antagonist is administered subcutaneously once weekly at a fixed dose of 800 mg. In some embodiments, the FcRn antagonist is administered subcutaneously once weekly at a fixed dose of 1000 mg. In some embodiments, the FcRn antagonist is administered subcutaneously once weekly at a fixed dose of 1200 mg. In some embodiments, the FcRn antagonist is administered subcutaneously once weekly at a fixed dose of 1250 mg. In some embodiments, the FcRn antagonist is administered subcutaneously once weekly at a fixed dose of 1500 mg. In some embodiments, the FcRn antagonist is administered subcutaneously once weekly at a fixed dose of 1750 mg. In some embodiments, the FcRn antagonist is efgartigimod.

In some embodiments, the FcRn antagonist is administered subcutaneously once weekly at a dose of about 10 mg/kg to about 25 mg/kg. In some embodiments, the FcRn antagonist is administered subcutaneously once weekly at a dose of about 10 mg/kg. In some embodiments, the FcRn antagonist is administered subcutaneously once weekly at a dose of about 15 mg/kg. In some embodiments, the FcRn antagonist is administered subcutaneously once weekly at a dose of about 20 mg/kg. In some embodiments, the FcRn antagonist is administered subcutaneously once weekly at a dose of about 25 mg/kg. In some embodiments, the FcRn antagonist is efgartigimod.

In some embodiments, the FcRn antagonist is administered subcutaneously once weekly at a dose of 10 mg/kg to 25 mg/kg. In some embodiments, the FcRn antagonist is administered subcutaneously once weekly at a dose of 10 mg/kg. In some embodiments, the FcRn antagonist is administered subcutaneously once weekly at a dose of 15 mg/kg. In some embodiments, the FcRn antagonist is administered subcutaneously once weekly at a dose of 20 mg/kg. In some embodiments, the FcRn antagonist is administered subcutaneously once weekly at a dose of 25 mg/kg. In some embodiments, the FcRn antagonist is efgartigimod.

In some embodiments, the FcRn antagonist is first administered intravenously and is subsequently administered subcutaneously. In some embodiments, the FcRn antagonist is first administered intravenously and is subsequently administered subcutaneously at fixed dose of 100 mg to 10,000 mg once weekly, once every 2 weeks, once every 3 weeks, once every 4 weeks, once monthly, or once every 6 weeks. In some embodiments, the FcRn antagonist is first administered intravenously and is subsequently administered subcutaneously at fixed dose of 1000 mg or 2000 mg once weekly, once every 2 weeks, once every 3 weeks, once every 4 weeks, once monthly, or once every 6 weeks. In some embodiments, the FcRn antagonist is efgartigimod.

In some embodiments, one or more doses of the FcRn antagonist are administered intravenously and subsequent doses of the FcRn antagonist are administered subcutaneously. In some embodiments, one or more doses of the FcRn antagonist are administered intravenously and subsequent doses of the FcRn antagonist are administered subcutaneously at fixed dose of 100 mg to 10,000 mg once weekly, once every 2 weeks, once every 3 weeks, once every 4 weeks, once monthly, or once every 6 weeks. In some embodiments, one or more doses of the FcRn antagonist are administered intravenously and subsequent doses of the FcRn antagonist are administered subcutaneously at fixed dose of 1000 mg or 2000 mg once weekly, once every 2 weeks, once every 3 weeks, once every 4 weeks, once monthly, or once every 6 weeks. In some embodiments, the FcRn antagonist is efgartigimod.

In some embodiments, the FcRn antagonist is administered for 6, 12, 26, 39, or 52 weeks or less. In some embodiments, the FcRn antagonist is administered for 26 weeks or less. In some embodiments, the FcRn antagonist is administered for 52 weeks or less. In some embodiments, the FcRn antagonist is administered for at least 6, 12, 26, 39, or 52 weeks. In some embodiments, the FcRn antagonist is administered for at least 26 weeks. In some embodiments, the FcRn antagonist is administered for at least 52 weeks.

In some embodiments, the FcRn antagonist is administered using a phased dosing schedule comprising multiple treatment cycles. In some embodiments, the phased dosing schedule comprises 2, 3, 4, 5, 6, 7, 8, 9, 10, or more treatment cycles. In some embodiments, each treatment cycle independently comprises or consists of administration of 1-5 doses of the FcRn antagonist within 1 month. In some embodiments, at least one treatment cycle comprises or consists of weekly administration of the FcRn antagonist for 4 weeks. In some embodiments, each treatment cycle comprises or consists of weekly administration of the FcRn antagonist for 4 weeks.

In some embodiments, the phased dosing schedule comprises a first treatment cycle and one or more subsequent treatment cycles. In some embodiments, the first treatment cycle and the one or more treatment cycles each independently comprise or consist of administration of 1-5 doses of the FcRn antagonist within 1 month. In some embodiments, the first treatment cycle comprises or consists of weekly administration of the FcRn antagonist for 4 weeks. In some embodiments, at least one of the one or more subsequent treatment cycles comprises or consists of weekly administration of the FcRn antagonist for 4 weeks. In some embodiments, each of the one or more subsequent treatment cycles comprise or consist of weekly administration of the FcRn antagonist for 4 weeks. In some embodiments, each of the one or more subsequent treatment cycles is administered to the subject based on clinical evaluation. In some embodiments, each of the one or more subsequent treatment cycles is administered ≥50 days from the start of the previous treatment cycle.

The instant disclosure also provides methods of vaccinating a subject having an antibody-mediated disorder, comprising administering a vaccine to the subject within about 2 months or about 1 month of administration of an FcRn antagonist to the subject. In some embodiments, the vaccine is administered to the subject within about 8 weeks, about 7 weeks, about 6 weeks, about 5 weeks, about 4 weeks, about 3 weeks, about 2 weeks, or about 1 week of administration of an FcRn antagonist to the subject. In some embodiments, the vaccine is administered to the subject within 2 months or 1 month of administration of an FcRn antagonist to the subject. In some embodiments, the vaccine is administered to the subject within 8 weeks, 7 weeks, 6 weeks, 5 weeks, 4 weeks, 3 weeks, 2 weeks, or 1 week of administration of an FcRn antagonist to the subject.

In some embodiments, the vaccine is administered to the subject before administration of an FcRn antagonist to the subject. In some embodiments, the vaccine is administered to the subject before administration of an initial dose of the FcRn antagonist.

In some embodiments, the vaccine is administered to the subject ≤4 weeks, ≤3 weeks, ≤2 weeks, or ≤1 week before administration of the FcRn antagonist to the subject. In some embodiments, the vaccine is administered to the subject ≤28 days, ≤27 days, ≤26 days, ≤25 days, ≤24 days, ≤23 days, ≤22 days, ≤21 days, ≤20 days, ≤19 days, ≤18 days, ≤17 days, ≤16 days, ≤15 days, ≤14 days, ≤13 days, ≤12 days, ≤11 days, ≤10 days, ≤9 days, ≤8 days, ≤7 days, ≤6 days, ≤5 days, ≤4 days, ≤3 days, ≤2 days, or ≤1 day before administration of the vaccine to the subject. In some embodiments, the vaccine is administered to the subject ≤24 hours before administration of the FcRn antagonist to the subject.

In some embodiments, the vaccine is administered to the subject about 4 weeks, about 3 weeks, about 2 weeks, or about 1 week before administration of the FcRn antagonist to the subject. In some embodiments, the vaccine is administered to the subject about 28 days, about 27 days, about 26 days, about 25 days, about 24 days, about 23 days, about 22 days, about 21 days, about 20 days, about 19 days, about 18 days, about 17 days, about 16 days, about 15 days, about 14 days, about 13 days, about 12 days, about 11 days, about 10 days, about 9 days, about 8 days, about 7 days, about 6 days, about 5 days, about 4 days, about 3 days, about 2 days, or about 1 day before administration of the FcRn antagonist to the subject. In some embodiments, the vaccine is administered to the subject 4 weeks, 3 weeks, 2 weeks, or 1 week before administration of the FcRn antagonist to the subject. In some embodiments, the vaccine is administered to the subject 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, or 1 day before administration of the FcRn antagonist to the subject. In some embodiments, the vaccine is administered to the subject before administration of the FcRn antagonist to the subject on the same day.

In some embodiments, the vaccine is administered to the subject after administration of an FcRn antagonist to the subject. In some embodiments, the vaccine is administered to the subject ≤2 months or ≤1 month after administration of the FcRn antagonist to the subject. In some embodiments, the vaccine is administered to the subject ≤8 weeks, ≤7 weeks, ≤6 weeks, ≤5 weeks, ≤4 weeks, ≤3 weeks, ≤2 weeks, or ≤1 week after administration of the FcRn antagonist to the subject. In some embodiments, the vaccine is administered to the subject ≤60 days, ≤59 days, ≤58 days, ≤57 days, ≤56 days, ≤55 days, ≤54 days, ≤53 days, ≤52 days, ≤51 days, ≤50 days, ≤49 days, ≤48 days, ≤47 days, ≤46 days, ≤45 days, ≤44 days, ≤43 days, ≤42 days, ≤41 days, ≤40 days, ≤39 days, ≤38 days, ≤37 days, ≤36 days, ≤35 days, ≤34 days, ≤33 days, ≤32 days, ≤31 days, ≤30 days, ≤29 days, ≤28 days, ≤27 days, ≤26 days, ≤25 days, ≤24 days, ≤23 days, ≤22 days, ≤21 days, ≤20 days, ≤19 days, ≤18 days, ≤17 days, ≤16 days, ≤15 days, ≤14 days, ≤13 days, ≤12 days, ≤11 days, ≤10 days, ≤9 days, ≤8 days, ≤7 days, ≤6 days, ≤5 days, ≤4 days, ≤3 days, ≤2 days, or ≤1 day after administration of the FcRn antagonist to the subject. In some embodiments, the vaccine is administered to the subject ≤24 hours after administration of the FcRn antagonist to the subject.

In some embodiments, the vaccine is administered to the subject about 2 months or about 1 month after administration of the FcRn antagonist to the subject. In some embodiments, the vaccine is administered to the subject about 8 weeks, about 7 weeks, about 6 weeks, about 5 weeks, about 4 weeks, about 3 weeks, about 2 weeks, or about 1 week after administration of the FcRn antagonist to the subject. In some embodiments, the vaccine is administered to the subject about 60 days, about 59 days, about 58 days, about 57 days, about 56 days, about 55 days, about 54 days, about 53 days, about 52 days, about 51 days, about 50 days, about 49 days, about 48 days, about 47 days, about 46 days, about 45 days, about 44 days, about 43 days, about 42 days, about 41 days, about 40 days, about 39 days, about 38 days, about 37 days, about 36 days, about 35 days, about 34 days, about 33 days, about 32 days, about 31 days, about 30 days, about 29 days, about 28 days, about 27 days, about 26 days, about 25 days, about 24 days, about 23 days, about 22 days, about 21 days, about 20 days, about 19 days, about 18 days, about 17 days, about 16 days, about 15 days, about 14 days, about 13 days, about 12 days, about 11 days, about 10 days, about 9 days, about 8 days, about 7 days, about 6 days, about 5 days, about 4 days, about 3 days, about 2 days, or about 1 day after administration of the FcRn antagonist to the subject.

In some embodiments, the vaccine is administered to the subject 2 months or 1 month after administration of the FcRn antagonist to the subject. In some embodiments, the vaccine is administered to the subject 8 weeks, 7 weeks, 6 weeks, 5 weeks, 4 weeks, 3 weeks, 2 weeks, or 1 week after administration of the FcRn antagonist to the subject. In some embodiments, the vaccine is administered to the subject 60 days, 59 days, 58 days, 57 days, 56 days, 55 days, 54 days, 53 days, 52 days, 51 days, 50 days, 49 days, 48 days, 47 days, 46 days, 45 days, 44 days, 43 days, 42 days, 41 days, 40 days, 39 days, 38 days, 37 days, 36 days, 35 days, 34 days, 33 days, 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, or 1 day after administration of the FcRn antagonist to the subject. In some embodiments, the vaccine is administered to the subject after administration of the FcRn antagonist to the subject on the same day.

In some embodiments, the vaccine is administered to the subject on the same day as administration of the FcRn antagonist to the subject.

Dosing regimens for vaccines are known in the art and vary depending on the vaccine. One advantage of the instant invention is that recommended dosing regimens do not need to be altered in subjects undergoing FcRn antagonist treatment. Thus, in some embodiments, the vaccine is administered to the subject according to a recommended dosing regimen for the vaccine. In some embodiments, the vaccine is administered to the subject in a single dose. In some embodiments the vaccine is administered to the subject in two or more doses. In some embodiments, the vaccine is administered to the subject in two doses. In some embodiments, when the vaccine is administered to the subject in two or more doses, each dose may be administered more than about 1 week apart, more than about 2 weeks apart, more than about 3 weeks apart, or more than about 4 weeks apart. In some embodiments, when the vaccine is administered to the subject in two or more doses, each dose may be administered more than 1 week apart, more than 2 weeks apart, more than 3 weeks apart, or more than 4 weeks apart. In some embodiments, when the vaccine is administered to the subject in two or more doses, each dose may be administered less than about 12 months apart, less than about 9 months apart, less than about 6 months apart, less than about 3 months apart, less than about 2 months apart, or less than about 1 month apart. In some embodiments, when the vaccine is administered to the subject in two or more doses, each dose may be administered less than 12 months apart, less than 9 months apart, less than 6 months apart, less than 3 months apart, less than 2 months apart, or less than 1 month apart.

In some embodiments, when the vaccine is administered to the subject in two or more doses, each dose may be administered from about 1 week to about 52 weeks apart. In some embodiments, when the vaccine is administered to the subject in two or more doses, each dose may be administered from about 2 to about 52, about 3 to about 52, about 4 to about 52, about 6 to about 52, about 8 to about 52, about 10 to about 52, about 12 to about 52, about 16 to about 52, about 20 to about 52, about 24 to about 52, about 26 to about 52, about 28 to about 52, about 32 to about 52, about 36 to about 52, about 40 to about 52, about 44 to about 52, or about 48 to about 52 weeks apart.

In some embodiments, when the vaccine is administered to the subject in two or more doses, each dose may be administered from 1 week to 52 weeks apart. In some embodiments, when the vaccine is administered to the subject in two or more doses, each dose may be administered from 2 to 52, 3 to 52, 4 to 52, 6 to 52, 8 to 52, 10 to 52, 12 to 52, 16 to 52, 20 to 52, 24 to 52, 26 to 52, 28 to 52, 32 to 52, 36 to 52, 40 to 52, 44 to 52, or 48 to 52 weeks apart.

In some embodiments, when the vaccine is administered to the subject in two or more doses, each dose may be administered from about 1 to about 48, about 1 to about 44, about 1 to about 40, about 1 to about 36, about 1 to about 32, about 1 to about 28, about 1 to about 26, about 1 to about 24, about 1 to about 20, about 1 to about 16, about 1 to about 12, about 1 to about 10, about 1 to about 8, about 1 to about 6, about 1 to about 4, about 1 to about 3, or about 1 to about 2 weeks apart.

In some embodiments, when the vaccine is administered to the subject in two or more doses, each dose may be administered from 1 to 48, 1 to 44, 1 to 40, 1 to 36, 1 to 32, 1 to 28, 1 to 26, 1 to 24, 1 to 20, 1 to 16, 1 to 12, 1 to 10, 1 to 8, 1 to 6, 1 to 4, 1 to 3, or 1 to 2 weeks apart.

In some embodiments, administration of the vaccine to the subject elicits a protective immune response. As used herein, a protective immune response is defined as enhanced immunity to a pathogen after exposure to antigen from the pathogen. Such exposure to antigen may come from exposure to the pathogen itself and/or from administration of a vaccine comprising the pathogenic antigen. A protective immune response to a vaccine may be due to the presence of circulating antibody (humoral immunity), the actions of sensitized T-lymphocytes (cell-mediated immunity), the presence of secretory IgA on mucosal surfaces (mucosal immunity), or a combination of these factors. In some embodiments, the protective immune response is humoral immunity. As used herein, a protective threshold is an antibody titer or level that is sufficient to protect against infection and/or disease upon exposure to pathogen.

In some embodiments, the vaccine comprises one or more pathogenic antigens. In some embodiments, IgG antibody titers and/or levels against one or more of the pathogenic antigens are maintained at or above a protective threshold following administration of the FcRn antagonist to the subject. Such protective thresholds are known in the art. An exemplary protective threshold for anti-tetanus toxoid (TT) is defined as >0.1 IU/mL. An exemplary protective threshold for anti-varicella zoster virus (VZV) is defined as >150 IU/mL. An exemplary protective threshold against pneumococcal serotypes included in pneumococcal conjugate vaccines (e.g., PCV13) is an IgG antibody concentration of ≥350 ng/mL.

Anti-influenza vaccine titers are typically measured by either using a hemagglutination inhibition (HI) assay or a single radial hemolysis (SRH) assay. Antibody titers, also referred to as neutralizing antibody titers, are generally presented as a ratio. For example, a ratio of 1:200 indicates that diluting one part of the blood sample to 200 parts of the diluent solution finally leads to an undetectable antibody level in the blood sample. Exemplary anti-influenzae protective thresholds using these assays are an HI titer of ≥1:40 or an SRH zone area of 25 mm².

Protective thresholds for SARS-CoV-2 IgG antibody titers have not yet been established. In some embodiments, receptor-binding domain (RBD) IgGs specific for SARS-CoV-2 S-protein are assessed using a 5-plex Luminex Assay (Q2 Solutions, Morrisville, NC). Serum concentrations of the IgG antibodies specific to a panel of five different antigens used as capture molecules: spike protein S2 (S2), nucleocapsid (N), spike protein S1 (S1), full-length protein S (S1+S2 extra cellular domain), and receptor-binding domain (RBD) can be evaluated, for example, in a multiplex bead assay. Since absolute antibody concentration in the serum cannot be quantified using such assays, reference pooled human serum can be used to calculate relative titers, which are reported in arbitrary units per milliliter (AU/mL). In some embodiments, convalescent plasma is used to serve as reference values. In some embodiments, the protective threshold is an anti-SARS-CoV-2 Spike protein receptor-binding domain (S-RBD) IgG level of ≥620.2 AU/mL. In some embodiments, the protective threshold is an anti-S-RBD IgG level of ≥82.82 AU/mL. In some embodiments, the protective threshold is an anti-S-RBD IgG level of ≥50 AU/mL, ≥75 AU/mL ≥100AU/mL, ≥150AU/mL, ≥200 AU/mL, ≥250 AU/mL, ≥300 AU/mL, ≥350 AU/mL, ≥400 AU/mL, ≥450 AU/mL, ≥500 AU/mL, ≥600 AU/mL, ≥700 AU/mL, ≥800 AU/mL, ≥900 AU/mL, ≥1000 AU/mL, ≥1500 AU/mL, ≥2000 AU/mL, ≥3000 AU/mL, ≥4000 AU/mL, or ≥5000 AU/mL.

In some embodiments, IgG antibody titers against one or more of the pathogenic antigens are maintained at or above a baseline value following administration of an FcRn antagonist to the subject. In some embodiments, the baseline value is an IgG antibody titer against the one or more pathogenic antigens in the subject before administration of the FcRn antagonist to the subject. In some embodiments, the subject has an antigen-specific IgG response following subsequent antigen challenge. In some embodiments, the subject shows at least about a 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 40-fold, or 50-fold increase in antigen-specific IgG following a second administration of the vaccine. In some embodiments, the subject shows at least about a 4-fold increase in antigen-specific IgG following a second administration of the vaccine. In some embodiments, the subject shows at least about a 20-fold increase in antigen-specific IgG following a second administration of the vaccine. In some embodiments, the subject shows at least a 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 40-fold, or 50-fold increase in antigen-specific IgG following a second administration of the vaccine. In some embodiments, the subject shows at least a 4-fold increase in antigen-specific IgG following a second administration of the vaccine. In some embodiments, the subject shows at least a 20-fold increase in antigen-specific IgG following a second administration of the vaccine. In some embodiments, the antigen-specific IgG is an anti-S-RBD IgG.

### EXAMPLES

The following examples are offered by way of illustration, and not by way of limitation.

### Example 1: Effects of Efgartigimod Treatment on Humoral and Cellular Immune Response: Analysis of T-Cell-Dependent Antibody Response in Cynomolgus Monkeys

Efgartigimod is a human IgG1 antibody Fc-fragment that reduces IgG, including pathogenic IgG autoantibody levels through FcRn blockage. Owing to the novel mechanism of action of efgartigimod, its impact on adaptive humoral and cellular immune responses to antigen challenge warrants further investigation.

The objective of the present study was to determine whether treatment with efgartigimod in cynomolgus monkeys had an impact on humoral and/or cellular immune responses to keyhole limpet hemocyanin (KLH), a prototypical T-cell-dependent antigen.

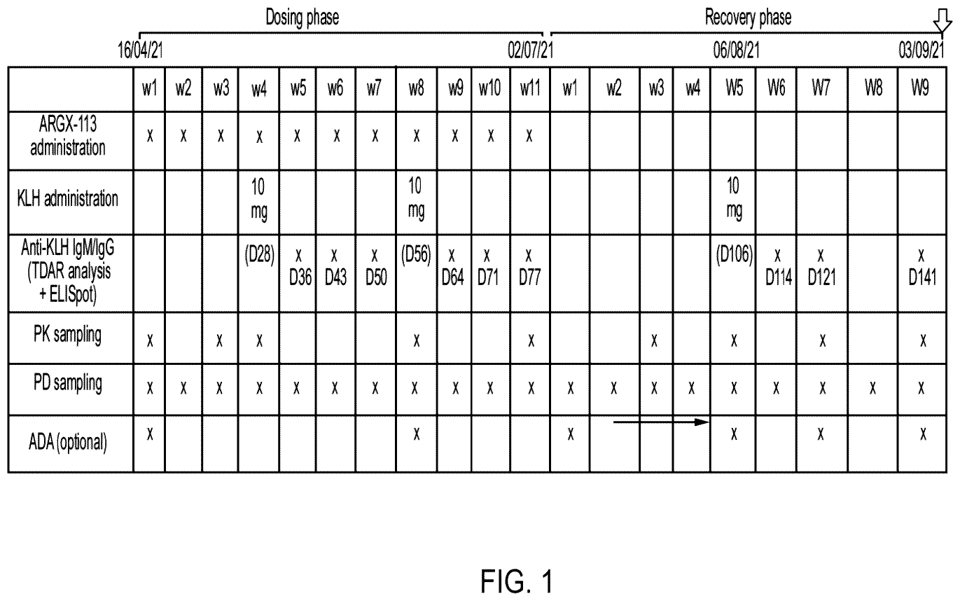

Six male and 6 female cynomolgus monkeys were divided 1:1 to receive either intravenous 100 mg/kg efgartigimod or vehicle control once weekly for 11 weeks, followed by a 9-week recovery period. KLH challenges (10 mg each) occurred at weeks 4 and 8, with an additional challenge at week 5 of the recovery period. A schematic of the study design is shown in FIG. 1.

In this study, a clear anti-KLH IgG and IgM induction was observed after KLH immunization in both groups.

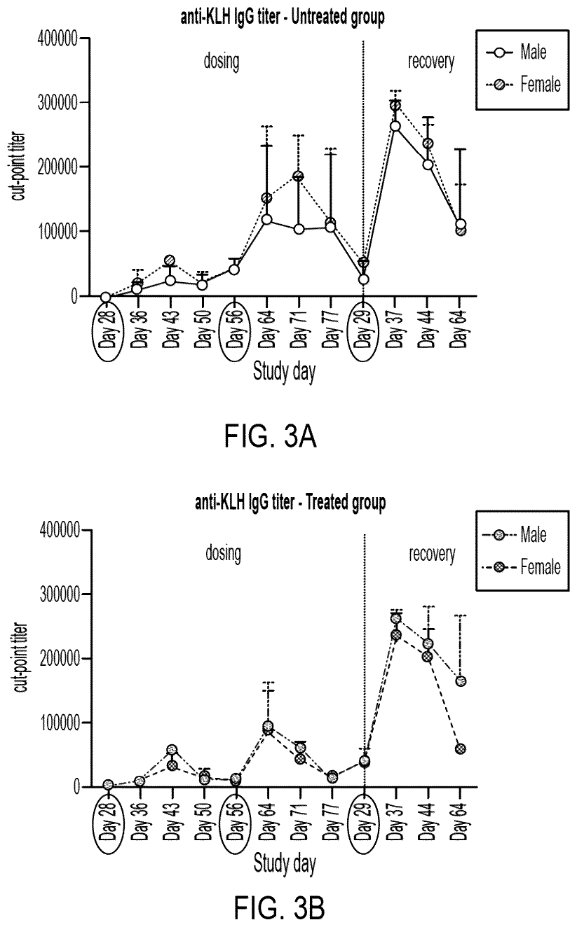

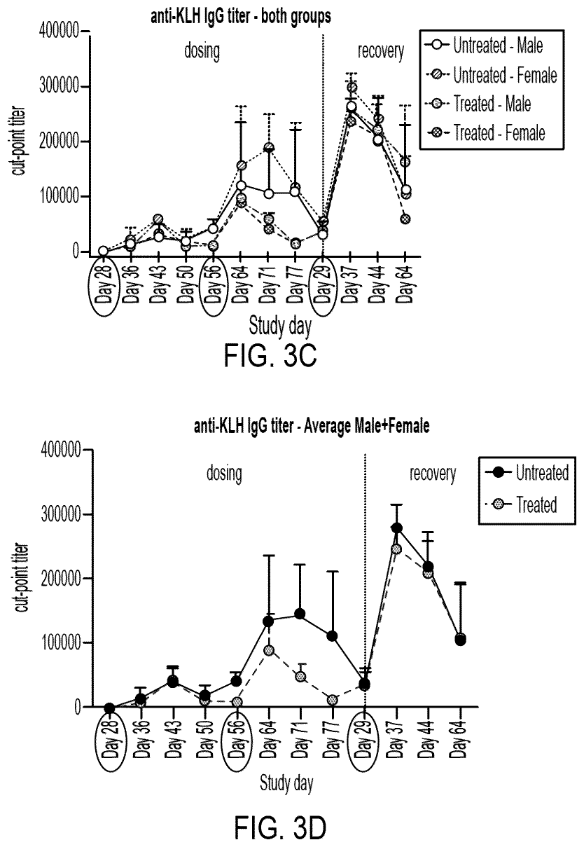

As shown in FIGS. 3A-3D, broadly similar anti-KLH IgG titers were generally noted in efgartigimod-treated animals for the duration of the dosing and recovery phases, compared with controls. The only significant difference observed was a decreased mean of the anti-KLH IgG antibody response on days 71 and 77 of the dosing phase (approximately 4- and 8-fold in females; 2- and 9-fold in males, respectively) following the second KLH immunization on day 56 in efgartigimod-treated animals, compared to respective controls.

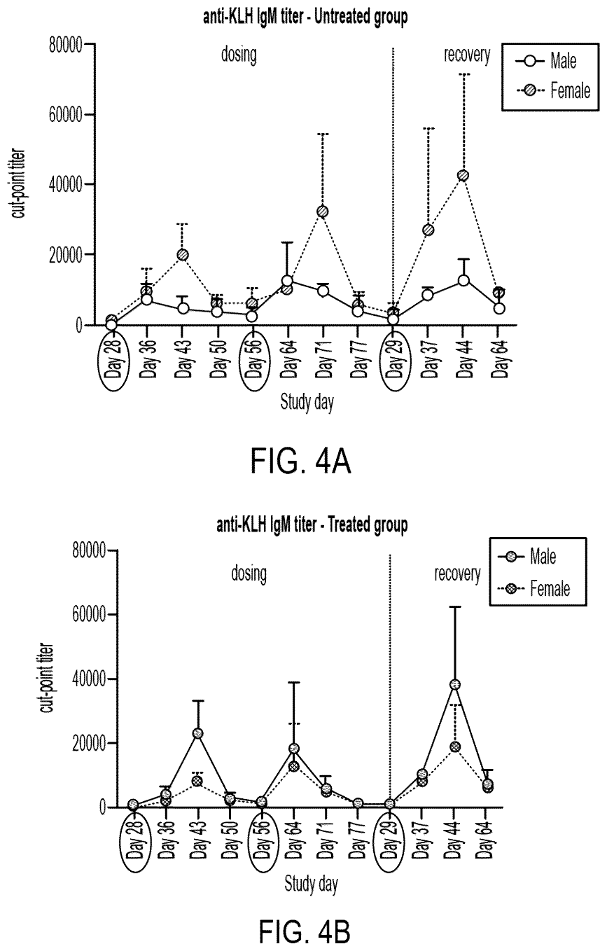

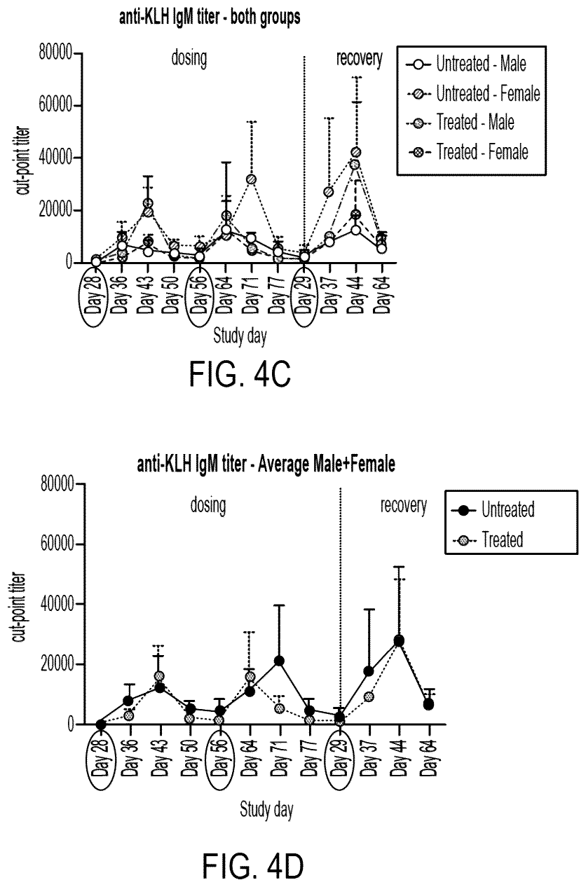

Similar anti-KLH IgM titers were noted in efgartigimod-treated animals throughout the dosing or recovery phase, compared to controls, as shown in FIGS. 4A-4D. A similar positive T-cell response (i.e., interferon-gamma secretion) to the immunizing agent KLH was also observed in both groups.

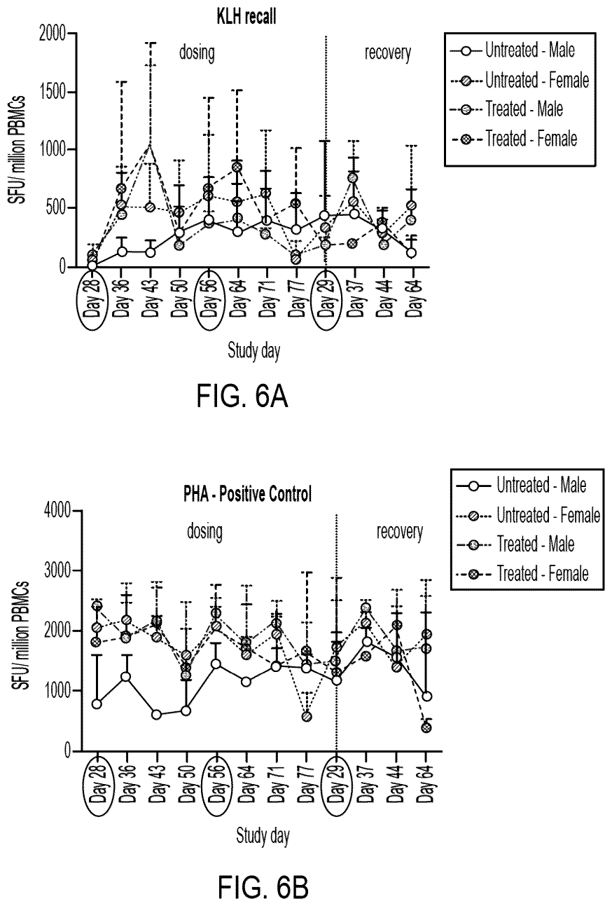

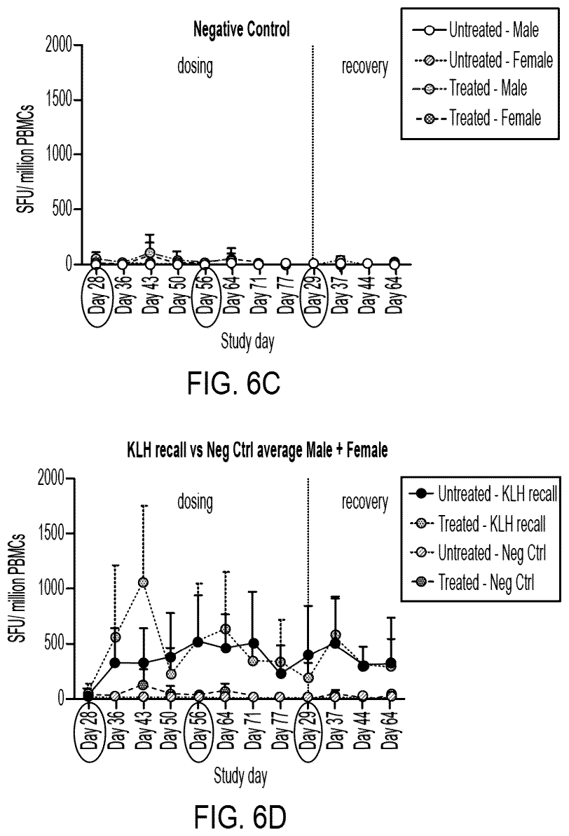

KLH-specific cellular responses were observed after KLH immunizations and maintained during the dosing and recovery phase, comparable with responses in the control group (FIGS. 6A & 6D). Responses to PHA stimulation served as a positive control for the functionality of PBMCs for all animals (FIG. 6B) and negative controls are shown in FIG. 6C.

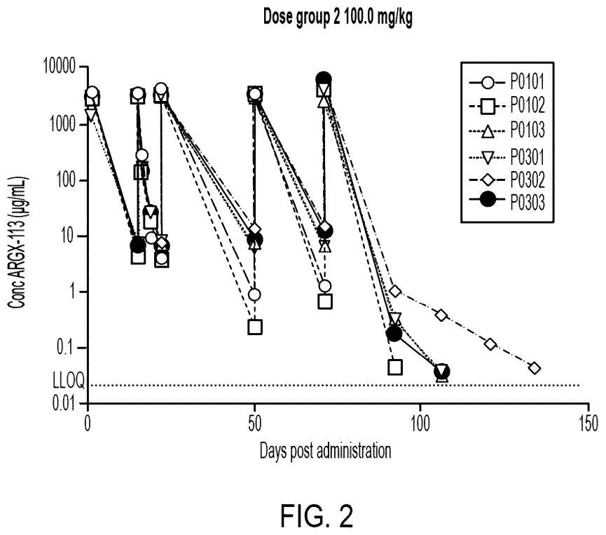

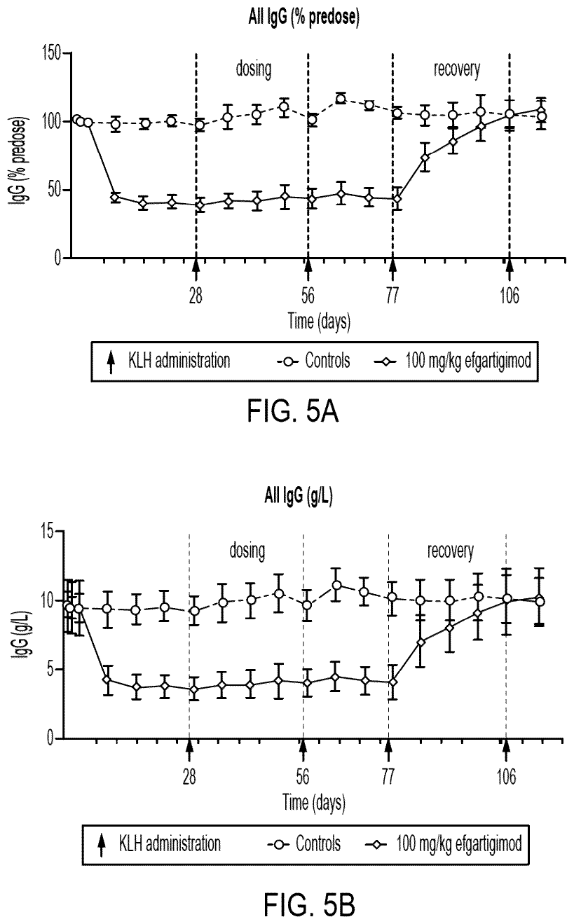

Consistent with the mode of action of efgartigimod, and as shown in FIGS. 5A-5B, total IgG levels decreased from day 8 through day 77 in efgartigimod-treated animals (average 0.4-fold change with respect to average pre-treatment levels), before returning to pre-dose levels by day 38 of the recovery phase. The pharmacokinetic profile (FIG. 2) in the efgartigimod-treated group was consistent with previous studies in cynomolgus monkeys.

These data showed that efgartigimod treatment did not affect the antigen-specific activation of the immune response or T-cell response in cynomolgus monkeys. A clear antigen-specific IgG response was mounted under efgartigimod treatment, and an increased production of antigen-specific IgG in response to stimuli was seen. In the context of vaccination or infection, efgartigimod treatment is not expected to impair generation of immune responses.

### Example 2: Effect of FcRn Antagonism on Protective Antibodies and to Vaccines in IgG-Mediated Autoimmune Diseases Pemphigus and Generalized Myasthenia Gravis

### A. Introduction

Antagonism of FcRn by efgartigimod has been studied in several autoimmune diseases mediated by IgG as a therapeutic approach to remove pathogenic IgGs. Whereas reduction of pathogenic titers has demonstrated efficacy in multiple autoimmune diseases, reducing total IgG could potentially increase infection risk in patients receiving FcRn antagonists. In this study, we sought to investigate the impact of efgartigimod treatment on humoral immune responses in Pemphigus and gMG. In patients with Pemphigus treated with efgartigimod for up to 34 weeks, we examined levels of protective antibodies associated with prior vaccinations or infection. In patients with gMG who participated in ADAPT and the corresponding open-label extension (ADAPT+), we analyzed humoral responses among those vaccinated to influenza, pneumococcus, or SARS-CoV-2 during the trial.

### B. Methods

### Study Designs

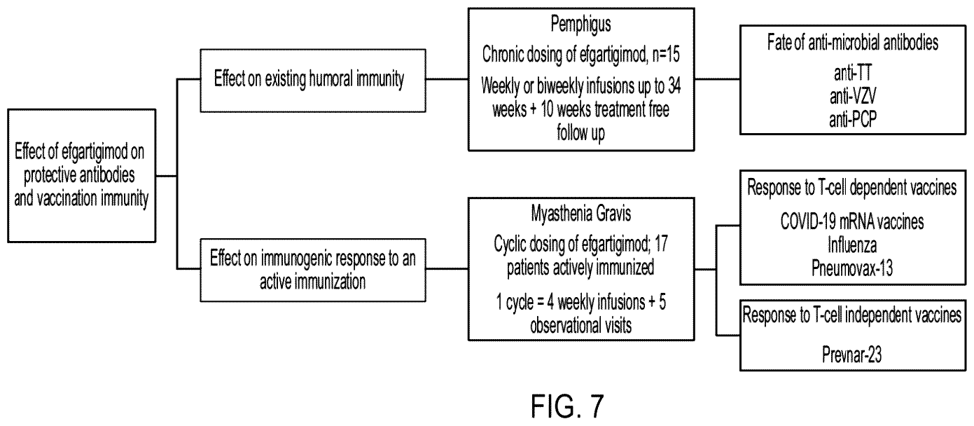

The present study consisted of two posthoc analyses of data from a phase 2, open-label trial of efgartigimod in patients with Pemphigus (NCT03334058) and from ADAPT, a phase 3, randomized, double-blind, placebo-controlled trial of efgartigimod in gMG (NCT03669588) and its open-label extension (ADAPT+; NCT03770403), as illustrated in FIG. 7.

### Pemphigus Trial

The Pemphigus phase 2 open-label study was an adaptive study to learn the conditions of use of efgartigimod in Pemphigus. This analysis involved 15 patients participating in cohort 4 of the phase 2 Pemphigus trial (see Table 8).

TABLE 8
Characteristics of patients with pemphigus who participated
in cohort 4 of the open-label study of efgartigimod

| | | | | Prednisone | | |
|---|---|---|---|---|---|---|
| | | Diagnosis, | | equivalent dose at | Exposure to | Protective |
| Patient | Gender, age | Pemphigus | Pemphigus | baseline | study drug | antibodies |
| no. | (years) | Type | Disease History | [mg/kg/day] | [days] | evaluated |
| 1 | F, 63 | PF | Relapsing | 0.28 | 247 | Anti-VZV |
| 2 | F, 48 | PV M | Relapsing | 0.28 | 232 | Anti-TT |
| 3 | M, 57 | PF | Relapsing | 0.24 | 240 | Anti-PCP |
| 4 | F, 42 | PF | Newly diagnosed | 0.31 | 232 | |
| 5 | F, 62 | PV M | Relapsing | 0.37 | 240 | |
| 6 | M, 66 | PV MC | Relapsing | 0.10 | 216 | |
| 7 | F, 85 | PF | Relapsing | 0.22 | 233 | |
| 8 | M, 47 | PV MC | Newly diagnosed | 0.31 | 237 | |
| 9 | M, 67 | PF | Newly diagnosed | 0.06 | 232 | |
| 10 | F, 36 | PF | Relapsing | 0.40 | 232 | |
| 11 | M, 58 | PV C | Newly diagnosed | 0.23 | 235 | |
| 12 | F, 66 | PV MC | Newly diagnosed | 0.33 | 170 | |
| 13 | F, 51 | PF | Newly diagnosed | 0.32 | 118 | |
| 14 | M, 22 | PV MC | Newly diagnosed | 0.33 | 29 | |
| 15 | M, 30 | PV C | Relapsing | 0.61 | 28 | |

PV, pemphigus vulgaris; PF, pemphigus foliaceus; M, mucosal-dominant; MC, mucocutaneous; C, cutaneous; VZV, varicella zoster virus; TT, tetanus toxoid; PCP, pneumococcal capsular polysaccharide.

In earlier cohorts, dosing every 4 weeks was insufficient to maintain autoantibody suppression and disease activity, while with weekly and alternate-week dosing, sufficient autoantibody-suppression to afford clinical benefit was observed. Patients treated in cohort 4 were dosed with weekly efgartigimod until reaching end of consolidation (EoC, the time at which approximately 80% of blisters have healed and no new blisters developed for at least two weeks) and then dosed on alternative weeks up to 34 weeks. These patients exhibited the strongest and the longest IgG suppression, thus represent the most relevant group for vaccination immunity assessment. All patients in this group had a confirmed diagnosis of PV or PF (by positive direct immunofluorescence showing IgG deposits on the keratinocytes cell surface, positive indirect immunofluorescence on monkey esophagus, and/or positive Dsg-1/3 enzyme linked immunosorbent assay) and mild to moderate disease defined by a Pemphigus disease area index (PDAI) <45 at baseline. Newly diagnosed as well as relapsing patients were eligible. These participants received intravenous efgartigimod 25 mg/kg with frequency as described above. Newly diagnosed patients, and relapsing patients who were off-therapy, also received prednisone 20 mg/day at baseline; those already taking prednisone continued receiving the tapered dose at which relapse occurred. Oral prednisone could be tapered from EoC. Full design details and study results have been published (Goebeler et al., Br J Dermatol. 2022; 186(3):429-439). History of vaccination within the last 4 weeks prior to baseline visit, or with a planned vaccination during the study, with the exception of seasonal vaccination (e.g., influenza vaccine), was an exclusion criterion. Serum titers of protective vaccine antibodies against tetanus toxoid (TT), varicella zoster virus (VZV), and pneumococcal capsular polysaccharide (PCP) were exploratory endpoints.

### ADAPT

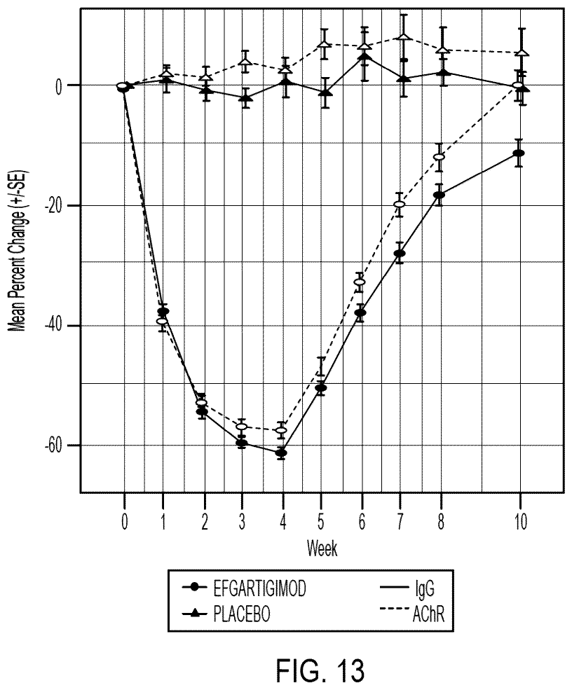

In ADAPT, 167 patients with gMG who on a stable dose of at least one MG therapy (acetylcholinesterase inhibitor, steroid, and/or non-steroidal immunosuppressant) were randomized 1:1 to efgartigimod IV 10 mg/kg or placebo. A full list of the inclusion and exclusion criteria can be found in the supplementary appendix of Howard et al., Lancet Neurol. 2021; 20(7):526-536. Treatments were administered as once weekly intravenous infusions for 4 weeks followed by a treatment-free period of at least 5 weeks. Additional treatment cycles were initiated based on an individualized dosing schedule according to clinical evaluation. After week 26, all participants could rollover into the ongoing, open-label extension (ADAPT+), during which they received efgartigimod according to an individualized cycle-based treatment schedule, including a minimum treatment-free period of 4 weeks. Significantly more patients in the efgartigimod group than in the placebo group had clinically meaningful improvements in the clinical patient-reported Myasthenia Gravis Activities of Daily Living and physician-reported Quantitative Myasthenia Gravis scores. The primary and some secondary endpoints required patients to have a clinically meaningful improvement in the associated outcome measure, and for this improvement to persist for at least 4 weeks. In AChR-Ab seropositive patients, there was a mean maximum reduction of 61.3% (SD 0.9) in total IgG 1 week after the fourth infusion in cycle 1 and levels returned to baseline by week 12. The reductions observed in AChR-Ab levels were similar and followed the same time course, with a mean maximal reduction of 57.6% (SD 1.3) obtained 1 week after the fourth infusion in cycle 1 as shown in the overlay of total IgG and anti-AChR-Ab levels in FIG. 13. Full trial results have previously been published (Howard et al., Lancet Neurol. 2021; 20(7):526-536).

The present analysis includes ADAPT participants who received an influenza or a pneumococcal vaccination during the 26-week double-blind treatment period or the ongoing open-label extension of the trial (ADAPT+) by the cutoff date of 10 Sep. 2020, or who received a COVID-19 vaccine during ADAPT+ prior to the cutoff date of 14 Jun. 2021. The ADAPT protocol permitted vaccinations, including inactivated or live-attenuated viruses if immunizations were given at least 48 h before or 48 h after efgartigimod administrations. No designated samples were collected to investigate established protective vaccine titers.

### Immunoglobulin Assays

### Pemphigus Trial

Serum titers of protective vaccine antibodies against TT, VZV, and serotypes of Streptococcus pneumoniae capsular polysaccharide were evaluated for all 15 patients enrolled in cohort 4 of the open-label study of efgartigimod in Pemphigus. Blood samples used in IgG assays were taken during each study visit from baseline through week 34 of the treatment period and continuing through the 10-week, treatment-free follow-up period. Total serum IgG was assessed using enzyme-linked immunosorbent assay (ELISA). Vaccine-specific serum IgG levels were measured using indirect enzyme immunoassay (EIA; Virotech, Rüsselsheim am Main, Germany) for TT (assay range 0.01-10 IU/mL), chemiluminescence immunoassay (CLIA; DiaSorin, Saluggia, Italy) for VZV (assay range 10-4000 mIU/mL), and EIA (The Binding Site Group, Birmingham, UK) for pneumococcal serotypes (assay range 3.3-270 mg/L).

Based on previous studies, protective titer thresholds were defined for anti-TT as >0.1 IU/mL and anti-VZV as >150 IU/L. Protective antibody thresholds for pneumococcal IgG have not been previously established for assays that utilize the 23-valent pneumococcal polysaccharide vaccine (PPSV23) antigen, and interlaboratory standardization of the PPSV23 assays has not been established. However, a 2- to 4-fold rise in IgG titers or levels >1.3 μg/mL for >50% to 70% of the serotypes one month after immunization with PPSV23 is generally considered a satisfactory post-vaccine (30 days) serologic response to pneumococcal polysaccharide vaccines.

### ADAPT

In ADAPT, pharmacodynamic analyses were conducted weekly for 8 weeks after initiation of each cycle and then every 2 weeks for 26 weeks during the double-blind phase as well as in a similar pattern for the first year in ADAPT+. Serum samples used for vaccine-specific IgG analysis were taken from each vaccinated patient before vaccination, approximately 4 weeks after vaccination, and between 56 and 239 days after vaccination. Total IgG was assessed using ELISA (SGS, Poitiers, France) and pathogenic AChR IgG antibodies were determined in AChR-Ab seropositive patients using a validated RIA assay (SGS, Poitiers, France). IgGs specific to the capsular polysaccharide of each of the 13 serotypes in the 13-valent pneumococcal conjugate vaccine (PCV13) were quantified with a Multiplex Luminex® assay (Covance/Labcorp, Princeton, NJ) and the same assay was used to assess the response to the 12 serotypes PPSV23 contains in common with PCV13. The World Health Organization recommends using an IgG antibody concentration of ≥350 ng/mL as the threshold of protection against pneumococcal serotypes included in pneumococcal conjugate vaccines.

Receptor-binding domain (RBD) IgGs specific for SARS-CoV-2 S-protein were assessed using a 5-plex Luminex Assay (performed at Q² Solutions, Morrisville, NC). Serum concentrations of the IgG antibodies specific to a panel of five different antigens used as capture molecules: spike protein S2 (S2), nucleocapsid (N), spike protein S1 (S1), full-length protein S (S1+S2 extra cellular domain), and RBD, were evaluated in a multiplex bead assay. Each of the recombinant antigens were covalently coupled to one of five distinct Luminex magnetic color-coded microspheres (“bead-conjugates”). Each microsphere can be recognized and quantified by dual-laser based fluorescent flow cytometry. A cut-off value for SARS-CoV-2 IgG antibody titers that defines clinical immunity has not yet been established. However, convalescent plasma was used for validation and samples at high, medium, and low concentrations (titers ranging from 620.2 to 4029.2 AU/ml) were evaluated to serve as reference values (see Table 9).

TABLE 9
Concentrations of convalescent plasma used as reference
values for SARS-CoV-2 IgG antibody titers

| | RBD Sample | | | | |
|---|---|---|---|---|---|
| Random effect | A | B | C | D* | E |
| Day, % CV | 0.0 | 0.0 | 0.0 | N/A | 0.0 |
| Operator, % CV | 0.0 | 1.5 | 0.0 | N/A | 0.0 |
| Residual, % CV | 5.8 | 14.6 | 5.8 | N/A | 8.7 |
| Total, % CV | 5.8 | 14.7 | 5.8 | N/A | 8.7 |
| Mean concentration (AU/mL) | 1371.4 | 2183.8 | 620.2 | 4029.2 | 923.6 |

*Only one result available. RBD variability and precision could not be determined from this sample. CV, coefficient of variation; RBD, receptor-binding domain; SARS-CoV-2, severe acute respiratory syndrome coronavirus 2.

A standard curve for each of the SARS-CoV-2 antigens was calculated using a 4-parameter logistic curve fit. Since the absolute antibody concentration in the serum cannot be quantified, the reference pooled human serum was used to calculate relative titers, which were reported in arbitrary units per milliliter (AU/mL). As only RBD IgG levels are relevant in the context of vaccination against SARS-CoV-2, only these levels were reported. The lower limit of quantification (LLOQ) was 23.6 AU/mL.

### Hemagglutination Inhibition Assay

Anti-influenza vaccine titers were determined using the hemagglutination inhibition assay (HI; Covance/Labcorp, Princeton, NJ). The reported neutralizing titer is the reciprocal of the highest dilution of serum that inhibits hemagglutination for a particular strain. A titer of ≥1:40 is considered seroprotective in adults. HI titers were evaluated against the influenza strains included in the 2019-2020 influenza season vaccines: A/Brisbane/02/2018 (H1N1)pdm09-like virus, A/Kansas/14/2017 (H3N2)-like virus, and B/Colorado/06/2017-like virus (B/Victoria/2/87 lineage) in the trivalent formulation and these three strains plus B/Phuket/3073/2013-like virus (B/Yamagata lineage) in the quadrivalent formulation.

### Data Analysis

Total serum IgG and protective antibody levels were summarized as mean percent change from baseline for each Ig measured or reported in units and plotted graphically by study days.

### C. Results

Total and Specific IgG Levels During Long-Term Efgartigimod Therapy Inpatients with Pemphigus

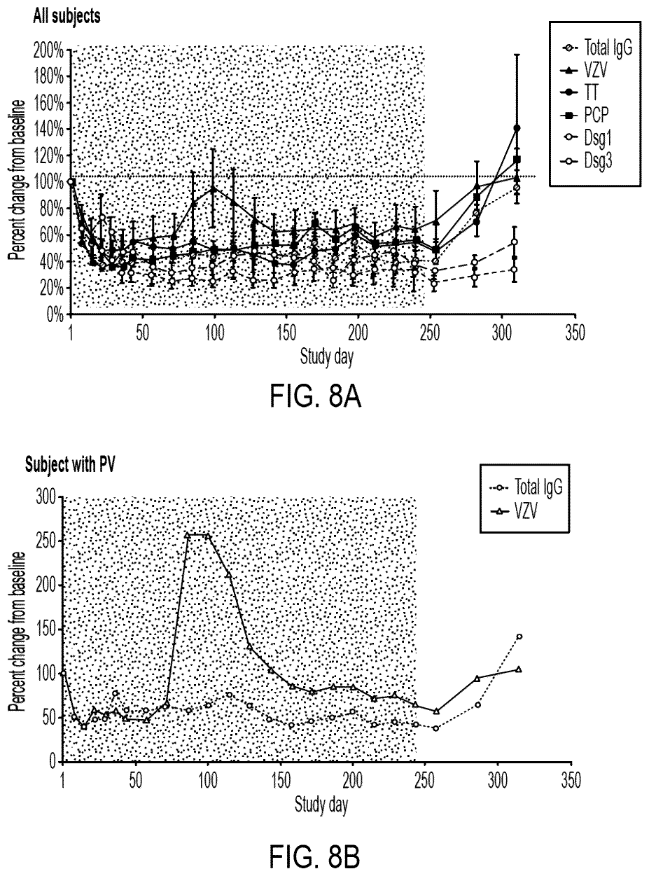

Results from the phase 2 trial of efgartigimod in Pemphigus have been previously reported (Goebeler et al., Br J Dermatol. 2022; 186(3):429-439; and Maho-Vaillant et al., Front Immunol. 2022; 13:863095). Briefly, disease control was achieved in 28 of 31 participants (90%) and complete clinical remission assessed only in cohort 3 and 4, was achieved in 14 of 22 participants (64%). Clinical improvements were strongly associated with the pharmacodynamic effect of efgartigimod. Over 34 weeks of efgartigimod treatment and 10 weeks of post-treatment follow-up, patients with Pemphigus exhibited a median 49% reduction from baseline in total serum IgG level after the first infusion and a median 66% reduction on Day 29 after 4 weekly infusions (FIG. 8A). Serum levels of anti-Dsg-1 and anti-Dsg-3 autoantibodies reached 50-60% reduction as long as efgartigimod was dosed. Anti-VZV and anti-PCP antibodies reached nadir between Days 22 and 36, whereas anti-TT IgG exhibited a somewhat slower decline. Total IgG and protective titers remained stably suppressed for as long as patients were dosed with biweekly efgartigimod. Following the last infusion (Day 238), a gradual return of total IgG and protective antibodies to baseline levels was observed. Pathogenic anti-Dsg autoantibodies showed more prolonged suppression as compared to total IgG and protective antibodies.

Twelve of 15 patients had baseline anti-TT IgG titers >0.1 IU/mL. Of those, two patients exhibited a transient decline of titers below 0.1 IU/mL, whereas 10 patients remained in the protective range throughout the study. We observed a transient and incidental drop in VZV-specific titers below the threshold of 150 IU/L in one subject whose baseline titer was 273 IU/L. No clinical signs of infection at times of maximal antibody suppression were observed.

During a period of stable total IgG suppression, an increase in anti-VZV antibodies was observed in two patients starting at approximately Day 36 for one patient and at Day 71 for the second patient (FIG. 8B), possibly due to an exposure to varicella or subclinical reactivation of latent VZV. No clinical signs of varicella infection were reported for either patient.

Response of Patients with gMG to T-Cell Dependent Vaccines

### COVID-19 Vaccination

During the ADAPT open-label extension (ADAPT+), seven patients received two doses of an mRNA COVID-19 vaccine by the cut-off date of 14 Jun. 2021 (see Table 10).

TABLE 10
Characteristics of patients with generalized myasthenia gravis (gMG)
who participated in ADAPT and were included in the analysis

| | Gender, | | Immuno- | ADAPT | Relevant | Vaccines received | | |
|---|---|---|---|---|---|---|---|---|
| Patient | age | BMI | suppressant | treatment | medical | COVID- | | |
| no. | (years) | (kg/m²) | therapy | group | history | 19 | Influenza | Pneumococcal |
| 1 | F, 20 | 32.8 | | Placebo | Hashimoto | BNT162b2 | NR | |
| 2 | M, 70 | 40.4 | MMF 500 mg | Placebo | | | NR | |
| | | | BID | | | | | |
| | | | Prednisone 20 | | | | | |
| | | | mg QD | | | | | |
| 3 | F, 47 | 23.9 | Prednisolone | Efgartigimod | Hepatic | | NR | |
| | | | 10 mg QD | | function | | | |
| | | | | | abnormality | | | |
| 4 | F, 56 | 45.2 | Prednisone 15 | Efgartigimod | T2D | | NR | |
| | | | mg QD | | | | | |
| 5 | F, 24 | 32.1 | Prednisone 15 | Efgartigimod | | | QIV | |
| | | | QOD | | | | | |
| | | | MMF 1 g | | | | | |
| | | | qAM + 1.5 g | | | | | |
| | | | qPM | | | | | |
| 6 | F, 46 | 32 | MMF 500 mg | Efgartigimod | | | QIV | PCV13 |
| | | | BID | | | | | |
| | | | Methylprednisolone | | | | | |
| | | | 4 mg QD | | | | | |
| 7 | F, 44 | 27.1 | Azathioprine | Efgartigimod | | | QIV | |
| | | | 50 mg BID | | | | | |
| | | | Methylprednisolone | | | | | |
| | | | 8 mg QD | | | | | |
| 8 | M, 76 | 22.7 | MMF 1000 | Efgartigimod | | | QIV | |
| | | | mg QD | | | | | |
| | | | Methylprednisolone | | | | | |
| | | | 8 mg QD | | | | | |
| 9 | F, 43 | 24.9 | Azathioprine | Efgartigimod | | | NR | |
| | | | 50 mg TID | | | | | |
| | | | Methylprednisolone | | | | | |
| | | | 10 mg QOD + 8 mg | | | | | |
| | | | QOD | | | | | |
| 10 | F, 55 | 38.4 | Mycophenolate | Efgartigimod | | | NR | |
| | | | 1.5 mg BID | | | | | |
| 11 | F, 40 | 34.2 | Azathioprine | Efgartigimod | Chronic | | NR | |
| | | | 150 mg QD + | | immune- | | | |
| | | | 100 mg QD | | suppressive | | | |
| | | | Prednisone 15 | | therapy | | | |
| | | | mg QD | | | | | |
| 12 | F, 79 | 24.1 | Prednisolone | Efgartigimod * | Diabetes, | BNT162b2 | | PPSV23 |
| | | | 5 mg QOD | | polymyalgia | | | |
| | | | Prednisolone | | rheumatica | | | |
| | | | 7.5 mg QOD | | | | | |
| 13 | M, 48 | 23.9 | Prednisolone | Efgartigimod * | No Medical | BNT162b2 | | |
| | | | 3 mg QD | | History | | | |
| | | | MMF 500 mg, | | | | | |
| | | | 5xd | | | | | |
| 14 | F, 76 | 34.5 | MMF 1 g BID | Efgartigimod * | T2D, CAD, | mRNA- | | |
| | | | Prednisone 2 | | CKD | 1273 | | |
| | | | mg QD | | | | | |
| 15 | F, 60 | 47.9 | MMF 1500 | Efgartigimod * | | BNT162b2 | | |
| | | | mg BID | | | | | |
| 16 | M, 65 | 35.6 | | Efgartigimod * | | BNT162b2 | | |
| 17 | M, 71 | 26.0 | | Efgartigimod * | | BNT162b2 | | |

*Received efgartigimod during ADAPT+ open-label extension. BID, twice daily; BMI, body mass index; CAD, coronary artery disease; CKD, chronic kidney disease; F, female; M, male; MMF, Mycophenolate mofetil; NR, received influenza vaccine but type not reported; PCV13, 13-valent pneumococcal conjugate vaccine; PPSV23, 23-valent pneumococcal polysaccharide vaccine; QD, once daily; QOD, every other day; QIV, quadrivalent influenza vaccine; T2D, type 2 diabetes.

Few blood samples were taken during ADAPT+, and no total IgG levels were analyzed. Therefore, we assessed the humoral response by measuring S-RBD IgGs from the limited sample set, and no correlation with total IgG levels was made.

None of the vaccinated patients experienced a known COVID-19 infection prior to receiving the vaccine or before the last post-vaccination sample was taken. One patient was immunized with mRNA-1273 (Moderna); the other six received BNT162b2 (Comirnaty; Pfizer), with a dosage interval of 16-40 days (see Table 9).

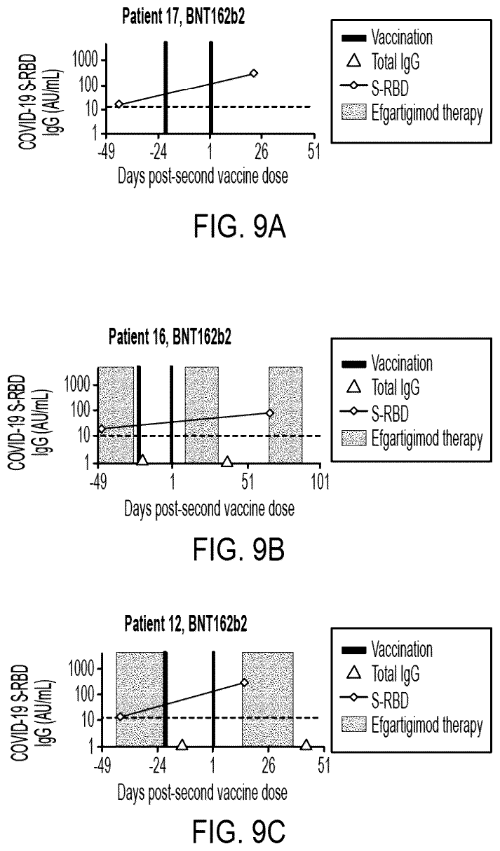

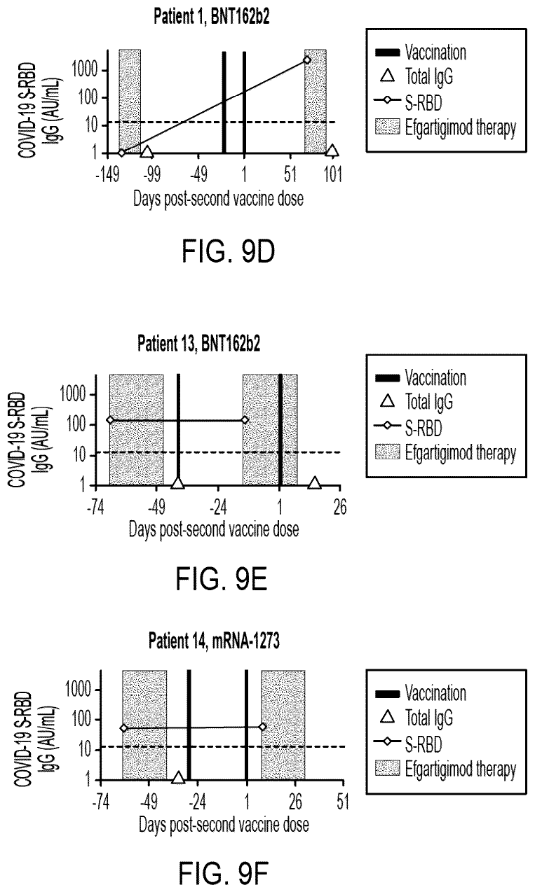

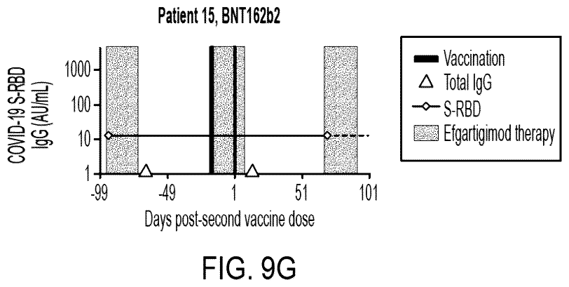

Patient 17 was enrolled in ADAPT+ but at the time samples were taken had not received efgartigimod treatment and was not receiving any immunosuppressant drugs. After receiving two doses of BNT162b2, the patient exhibited a 20-fold increase in S-RBD IgG levels (max titer of 301.78 AU/ml) of 21 days after receiving the second dose of vaccine (FIG. 9A).

Patient 16 was vaccinated with BNT162b2 1 week after the fourth infusion in an efgartigimod cycle, when total IgG levels are expected to be maximally reduced (represented by the arrows in FIGS. 9A-9G) and received another cycle of efgartigimod after the second dose of BNT162b2. This patient was not receiving any immunosuppressive therapy other than efgartigimod. When the post-vaccination sample was taken 66 days after the second BNT162b2 dose, a 4-fold increase in S-RBD IgG levels (max titer of 82.82 AU/ml) was observed (FIG. 9B).

In addition to efgartigimod, Patient 12 was treated concomitantly with prednisolone (5 mg and 7.5 mg orally, each once daily on alternating days). This patient received BNT162b2 at the end of an efgartigimod treatment period and showed a 20-fold increase in S-RBD IgG levels (max titer of 299.56 AU/ml) 15 days after the second dose (FIG. 9C).

Patient 1 (FIG. 9D), who was not receiving any immunosuppressive therapy other than efgartigimod, was vaccinated with BNT162b2 92 days post-infusion, when total IgG levels are expected to be unaffected, and exhibited a 63-fold increase in post-vaccination S-RBD IgG levels (max titer of 2233.65 AU/ml) 69 days after the second dose.

In the other three patients, no increase in titers was observed (FIGS. 9E-9G). Patient 15 had a body mass index of 47 kg/m², was treated with high doses of mycophenolate mofetil (1500 mg twice daily) in addition to efgartigimod, and received both BNT162b2 doses in the middle of an efgartigimod treatment cycle. No increase in S-RBD IgG levels was noted after the second dose of BNT162b2. The two other patients (Patients 13 and 14) also received mycophenolate mofetil along with prednisolone (see Table 3), but post-vaccination samples were collected at incorrect timings (no sample after the second dose for Patient 13 and sampling only 9 days after second dose for Patient 14), and therefore are not directly comparable.

### Influenza Vaccination

By 23 Jan. 2020, 11 patients with gMG participating in ADAPT had received an influenza vaccine, including two patients who were receiving placebo when they were vaccinated and nine patients who were receiving efgartigimod. Four of the efgartigimod recipients (Patients 5, 6, 7, and 8) received the quadrivalent influenza vaccine and the other five received an unknown influenza vaccine.

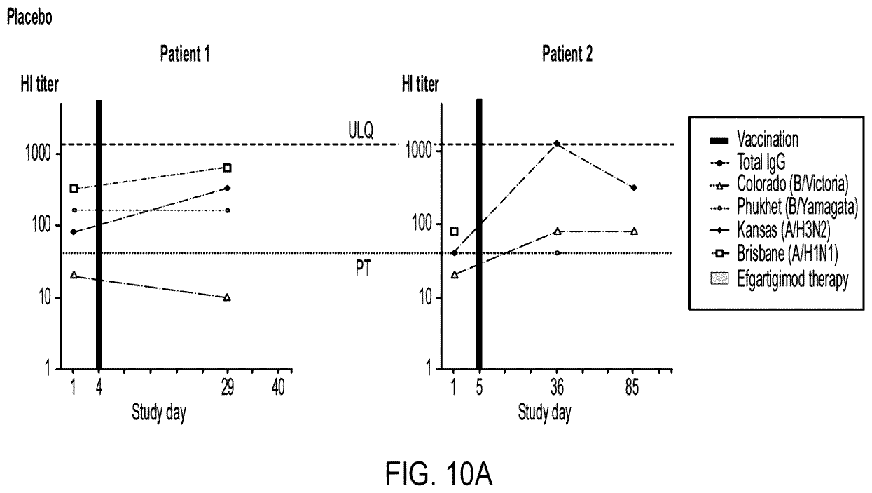

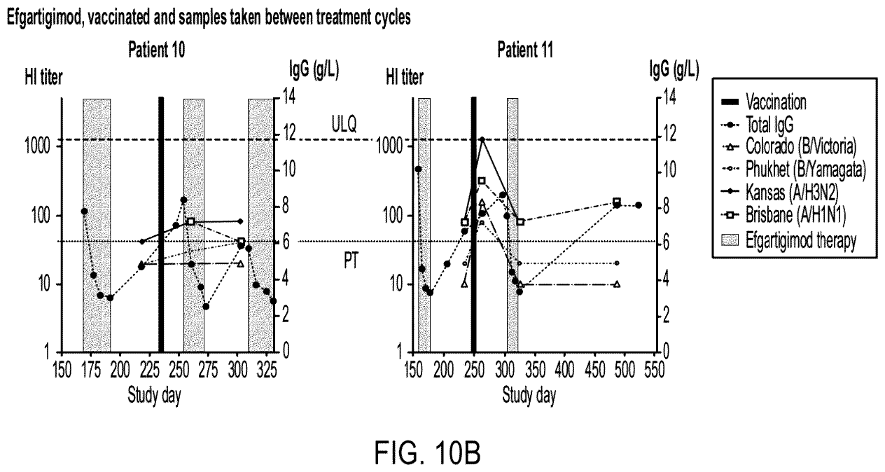

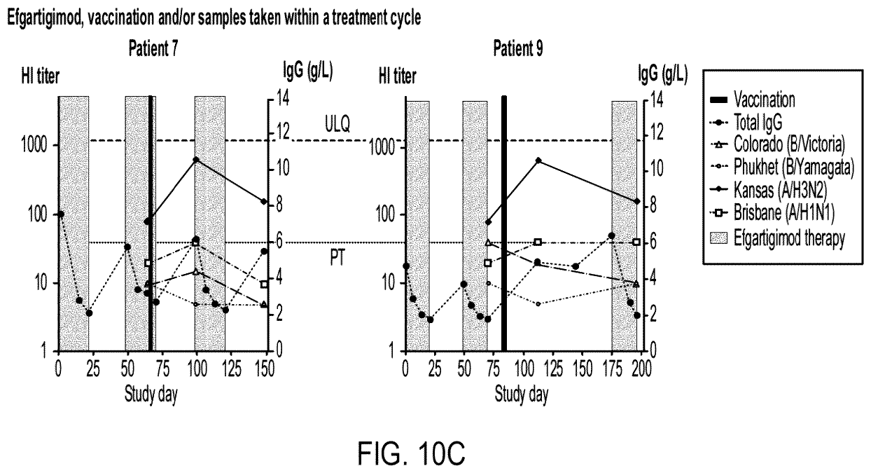

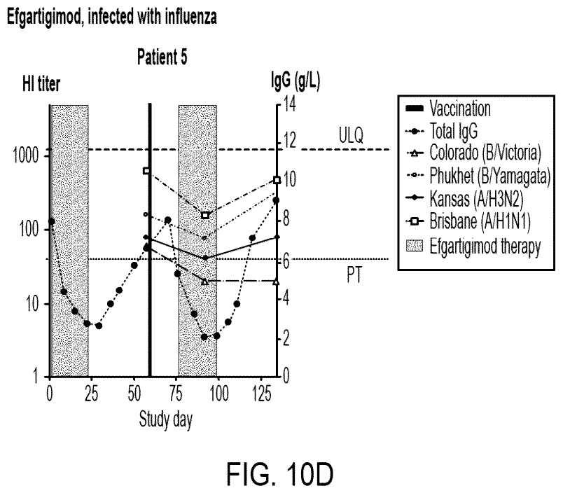

The immune response in the two placebo recipients exceeded the protective threshold (≥1:40) for most strains (FIG. 10A). In Patient 1, HI titers against A/H1N1 and A/H3N2 increased after vaccination, and those against B/Yamagata remained stable above the protective threshold (FIG. 10A, left graph). In Patient 2 (FIG. 10A, right graph), HI titers against A/H3N2 and B/Victoria rose above the protective threshold and remained there, while titers against B/Yamagata were stable at that level. Due to a missing sample, post-vaccination titers of A/H1N1 could not be analyzed.

As previously reported (Howard et al., Lancet Neurol. 2021; 20(7):526-536), total IgG and AChR-Ab IgG levels were significantly reduced in gMG patients treated with efgartigimod (FIGS. 10A-10D, 11A-11B, and 13). Individual anti-AChR-Ab levels are not shown in the figures as they follow the same time course as total IgG levels (FIG. 13). Patients 10 and 11 were vaccinated between efgartigimod treatment cycles (FIG. 10B). In Patient 10 (FIG. 10B, left graph), titers against A/H3N2 and B/Yamagata increased ˜2-fold 3 weeks after vaccination, when total IgG levels were comparable to pre-vaccination levels. Total IgG decreased during the efgartigimod cycle occurring between measurement of the two post-vaccination HI titers, yet the titers for A/H1N1, A/H3N2, and B/Yamagata were at or above the protective threshold 80 days after vaccination. In Patient 11 (FIG. 10B, right graph), the pre- and first post-vaccination samples were collected during a treatment-free period when IgG levels were not substantially reduced, and there was a clear post-vaccination immune response against all tested strains. When efgartigimod treatment was initiated 7 weeks after vaccination, total IgG levels were reduced by 61.5% at nadir. Influenza titers followed the same kinetics, decreasing to levels at or just above pre-vaccination levels but remaining above the protective threshold for A/H1N1 and A/H3N2. On day 486, total IgG levels were normalized, and titers against A/H3N2 and A/H1N1 were 2-fold higher than those measured before vaccination.

In Patients 7 and 9, the pre-vaccination samples were taken during an efgartigimod treatment cycle (FIG. 10C). Patient 7 (FIG. 10C, left graph) received the influenza vaccine near the end of an efgartigimod cycle, close to the IgG nadir. Eighty Days after vaccination, when total IgG levels were 2 times higher than those at the time of vaccination, titers against A/H3N2 had increased 8-fold and remained above protective levels after an additional cycle of efgartigimod. Patient 9 (FIG. 10C, right graph) was vaccinated 2 weeks after an efgartigimod cycle while total IgG levels were still reduced. One month after vaccination, when total IgG levels were 2.5-fold higher than pre-vaccination levels, HI titers against A/H3N2 had increased 8-fold and those against A/H1N1 increased to the protective threshold; titers against both strains remained at or above protective levels throughout a subsequent efgartigimod cycle.

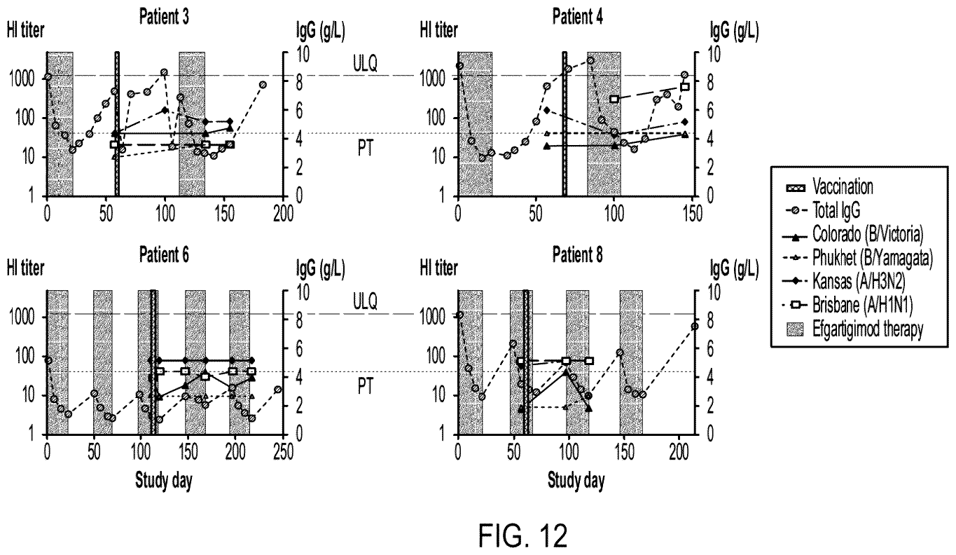

As shown in FIG. 12, Patient 6 showed no response to any of the strains tested, whereas titers remained >40 (the protective threshold) for other efgartigimod-treated subjects (Patients 3, 4, and 8), despite efgartigimod-induced IgG reduction.

Of the influenza vaccine recipients, only Patient 5 reported influenza-like illness. Influenza was confirmed with nasal swab, and the grade of the outcome was severe. The patient was diagnosed with influenza at Day 127 in the study, which coincided with the timing of the second post-vaccination sample (FIG. 10D). At this time point, the total IgG levels had returned to baseline, and influenza titers were above the protective threshold for all strains except B Victoria.

Among ADAPT participants who did not receive a 2019-2020 seasonal influenza vaccine during the study, 2/81 (2.5%) efgartigimod-treated patients and 3/81 (3.7%) of those receiving placebo reported influenza as an adverse event. In the open-label extension, 2/133 (1.5%) unvaccinated patients reported influenza.

### PCV13

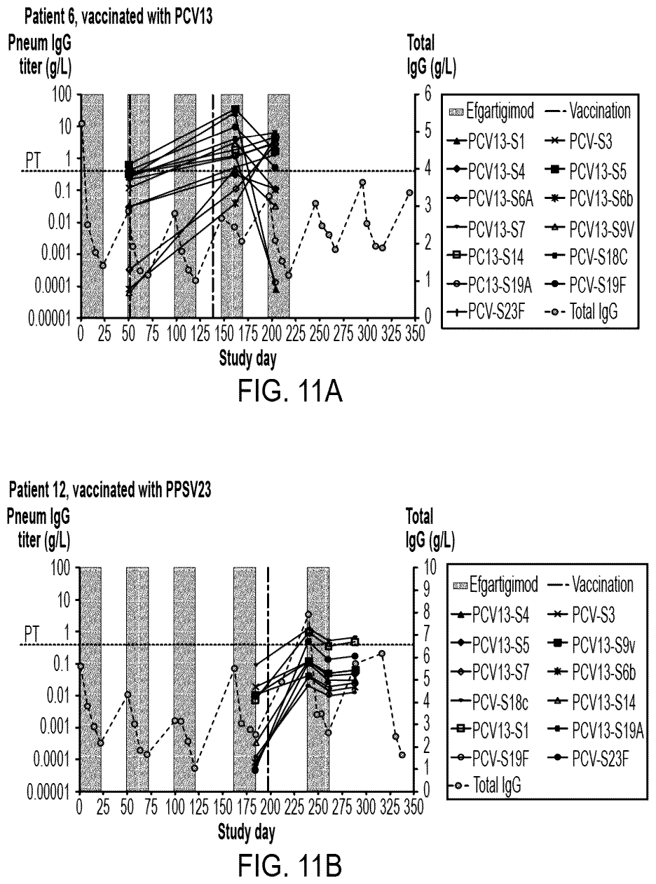

In addition to the influenza vaccine, Patient 6 randomized in the ADAPT study received two doses of PCV13 based on the determination of the treating physician: the first during an efgartigimod treatment cycle and the second dose in a treatment-free period (FIG. 11A). The pre-vaccination sample was collected at the start of the efgartigimod treatment cycle. A clear antibody response was seen toward all 13 strains. After the initiation of another treatment cycle, the titers decreased for some strains but remained above 350 ng/mL for 8 of 13 strains.

Response of Patients with gMG to T-Cell Independent Vaccine: PPSV23

Patient 12 was vaccinated with PPSV23 during the fourth dose in the cycle (FIG. 11B). Anti-pneumococcal titers were assessed against 12 of 23 strains included in the vaccine, and pre- to post-vaccination titer increases were observed ranging from 3.8- to 9183-fold. After a subsequent cycle, when total IgG levels were maximally reduced, anti-pneumococcal titers remained higher than pre-vaccination levels for all tested strains, with at least 4-fold increase compared to baseline for 11 of 12 assessed strains within this one patient. In general, titers for the tested strains remained substantially increased from baseline levels even during periods of reduced total IgG levels.

### D. Discussion

Antagonism of FcRn has been shown to be a potent therapeutic strategy to clear pathogenic autoantibodies in IgG-driven autoimmune disorders; however, the present study is the first to examine the effect on specific vaccine titers and humoral immune response to novel vaccine challenges in patients treated with the FcRn antagonist efgartigimod. Our data suggest that during efgartigimod treatment, patients can maintain the ability to mount an IgG response to antigen challenges and levels of protective antibody titers, whether induced naturally or by vaccines, closely follow total IgG reduction kinetics.

In the Pemphigus population, reductions in protective antibody titers for TT, VZV, and PCP paralleled the reduction in total IgG levels (FIGS. 8A-8B). The subsequent return to normal levels upon cessation of efgartigimod treatment indicated no negative effect on long-lived plasma cell (LLPC) responses. Based on phase 1 data, IgA and IgM levels should not be impacted and, in this study, patients also retained the capacity to produce antibodies against an ongoing infection during a phase of maximal IgG reduction induced by efgartigimod.

Immunosuppressive therapy is often utilized in both Pemphigus and gMG contributing to secondary immune deficiency and therefore an increased risk of infections, including COVID-19. The influence of disease pathology and immunosuppressive therapy are inseparable factors that collectively contribute to patients' vulnerability to infections. In ADAPT, efgartigimod was well tolerated, and the majority of infections (reported by 46% of efgartigimod-treated patients and 37% of those receiving placebo) were mild to moderate in severity (Howard et al., Lancet Neurol. 2021; 20(7):526-536). Similarly, no increased infection pattern was observed in an open-label trial of efgartigimod in Pemphigus (Goebeler et al., Br J Dermatol. 2022; 186(3):429-439). Upon efgartigimod treatment discontinuation, levels of total serum IgG quickly return to baseline levels. Low rates of infections documented in studies of antibody elimination by plasma exchange or immunoadsorption support the hypothesis that transient and selective reductions in IgG levels would be associated with a lower risk of infection than agents with broader immune actions.

Our analysis demonstrated that patients with gMG retained the ability to generate protective titers to vaccine challenge while receiving efgartigimod, regardless of the timing of vaccinations relative to efgartigimod cycles or the type of vaccine. Previous studies have shown that immune responses to influenza vaccination in patients with gMG were similar to those in healthy subjects, regardless of the use of some immunosuppressive therapies. In our study, an IgG response to T-cell dependent vaccines was observed in the majority (73.3%) of efgartigimod-treated patients, and in one patient to a T-cell independent vaccine, suggesting that FcRn antagonism did not interfere with immune responses to the vaccines studied. Similar numbers of unvaccinated patients in the efgartigimod and placebo groups of the ADAPT study were infected with influenza, suggesting efgartigimod did not necessarily increase susceptibility to influenza infection.

Broad immunosuppressants that affect T lymphocytes, B lymphocytes, or other components of the immune system, such as B-cell depleting agents, corticosteroids, abatacept, methotrexate, and mycophenolate mofetil have been reported to impair immune responses to SARS-CoV-2 vaccines and other vaccines; indeed, patients in this study who were treated with high doses of mycophenolate mofetil had a reduced IgG response to COVID-19 vaccination. In contrast, patients who received more selective agents such as TNF-inhibitors exhibited a robust serological response after mRNA COVID-19 vaccination. Efgartigimod also more selectively targets the immune system, reducing IgG levels while leaving other immune system components stated above that are necessary to mount significant immune responses largely untouched.

Animal data support the observation that effective immune responses can be mounted in the presence of FcRn antagonism and reduced IgG levels. Furthermore, subjects with genetic defects resulting in lack of functional FcRn maintained the ability to mount immune responses upon vaccination. While neither of these studies fully compare with transient pharmacologic FcRn antagonism, they provide insight on how the lack of functional FcRn can affect immune responses.

In conclusion, reduction of IgG titers via FcRn antagonism with efgartigimod did not impair the ability of the patients analyzed to generate new specific IgG responses, regardless of the timing of vaccinations. Immune titers typically increased whether vaccinations were given during or between efgartigimod cycles or when IgG levels were at nadir. Although vaccine-specific IgG levels were reduced proportionally to total IgG levels upon efgartigimod treatment, in general, they remained above the pre-vaccination levels and/or protective thresholds. Upon treatment discontinuation, total IgG levels returned to baseline, and protective titers were increased. These findings from this limited observational dataset suggest that FcRn antagonism does not prevent IgG production, with no impact on LLPCs, and protective antibody levels from vaccination can be maintained, even when total IgG levels are maximally reduced by efgartigimod.

### Example 3: Effect of FcRn Antagonism on COVID-19 Vaccination in Patients from the ADAPT+ Trial

The present Example pertains to the effect of efgartigimod therapy in 13 patients from the ADAPT/ADAPT+ trial (detailed above, in Example 2) who also received at least one dose of a COVID-19 vaccine.

Amongst these 13 patients, one patient received mRNA-1273 (Spikevax; Moderna), one patient received Ad26.COV2.S (Jcovden; Johnson & Johnson/Janssen), one patient received at least one dose of AZD1222 (Vaxzevria; AstraZeneca), nine patients received at least one dose of BNT162b2 (Comirnaty; Pfizer), and one patient received at least one dose of an unknown COVID-19 vaccine. Six of these patients received at least one dose of vaccine during an efgartigimod cycle.

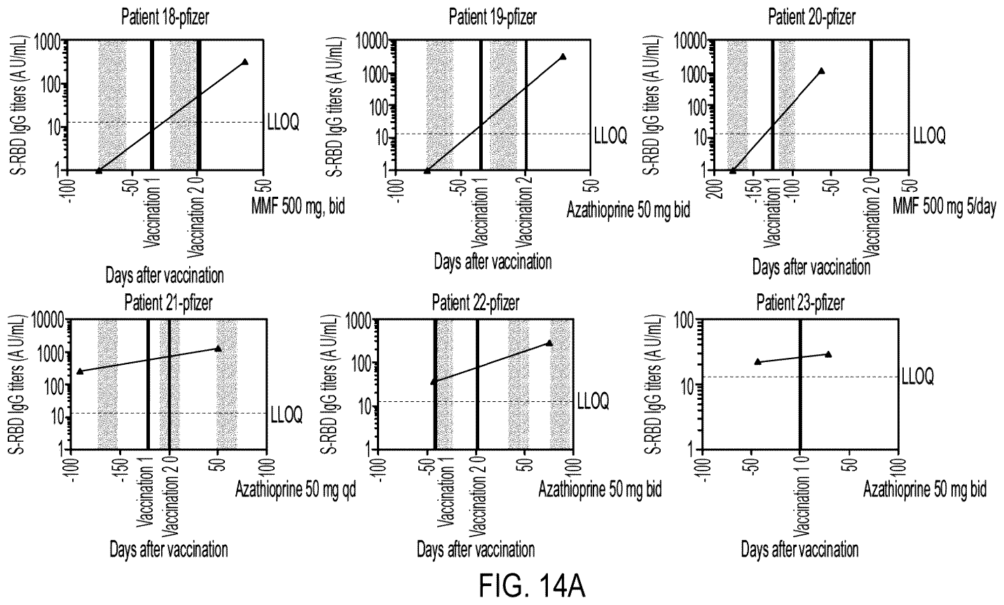

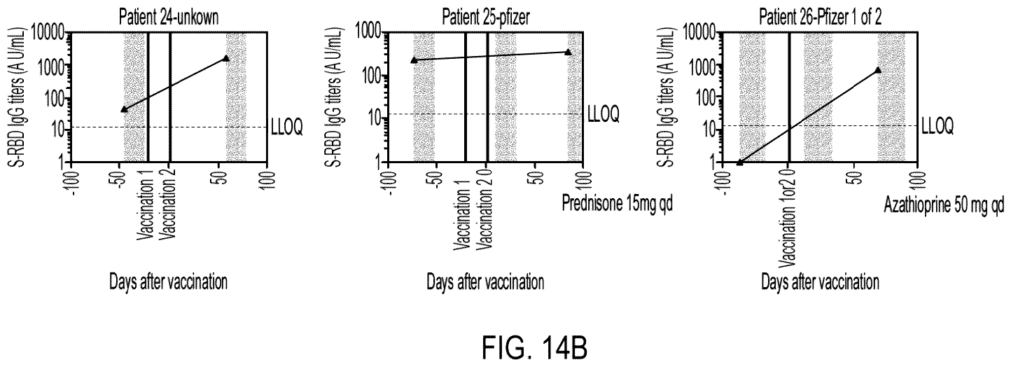

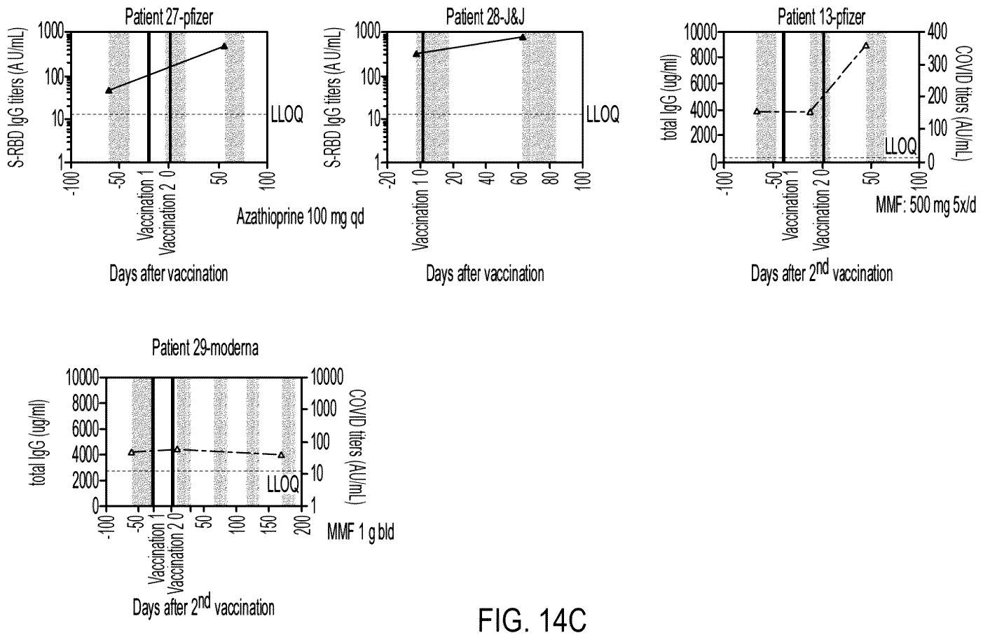

As shown in FIGS. 14A-14C, a large majority (11 out of 13) of the patients generated an IgG response against the SARS-CoV-2 S-protein receptor-binding domain (S-RBD). FIG. 14C also presents data from an additional timepoint for Patient 13, for whom earlier timepoints are shown in FIG. 9E. No conclusions could be drawn from the earlier S-RBD IgG data because of the lack of data after the second dose of vaccine. This new data confirms that Patient 13 did mount a humoral response to COVID-19 vaccination. Only two patients appear to have a questionable response (one receiving BNT162b2 (Patient 25-pfizer), the other mRNA-1273 (Patient 29-moderna)). These two patients were receiving additional therapeutic treatments: Patient 25-pfizer was receiving 15 mg/day of prednisone, and Patient 29-moderna was receiving mycophenolate mofetil (MMF) at 1 g twice a day; in both cases, both vaccine doses were given between efgartigimod cycles.

As in Example 2 above, reduction of IgG titers via FcRn antagonism with efgartigimod did not impair the ability of the patients analyzed to generate S-RBD specific IgG responses after administration of a COVID-19 vaccine, even when total IgG levels were reduced by efgartigimod.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

1. A method of reducing serum IgG levels in a subject having an antibody-mediated disorder, comprising administering an effective amount of a human neonatal Fc receptor (FcRn) antagonist to the subject within 4 weeks of administration of a vaccine to the subject.

2. A method of vaccinating a subject having an antibody-mediated disorder, comprising administering a vaccine to the subject within 4 weeks of administration of an FcRn antagonist to the subject.

3. The method of claim 1 or 2, wherein the FcRn antagonist is administered to the subject ≤4 weeks after administration of the vaccine to the subject.

4. The method of any one of claims 1-3, wherein the FcRn antagonist is administered to the subject ≤2 weeks or ≤1 week after administration of the vaccine to the subject.

5. The method of any one of claims 1-4, wherein the FcRn antagonist is administered to the subject ≤24 hours after administration of the vaccine to the subject.

6. The method of any one of claims 1-5, wherein an initial dose of the FcRn antagonist is administered to the subject after administration of the vaccine to the subject.

7. A method of reducing serum IgG levels in a subject having an antibody-mediated disorder, comprising administering an effective amount of an FcRn antagonist to the subject ≤2 months before administration of a vaccine to the subject.

8. A method of vaccinating a subject having an antibody-mediated disorder, comprising administering a vaccine to the subject ≤2 months after administration of an FcRn antagonist to the subject.

9. The method of any one of claims 1-8, wherein the FcRn antagonist is administered to the subject ≤4 weeks, ≤2 weeks, or ≤1 week before administration of the vaccine to the subject.

10. The method of any one of claims 1-9, wherein the FcRn antagonist is administered to the subject ≤24 hours before administration of the vaccine to the subject.

11. The method of any one of claims 1-10, wherein the FcRn antagonist is administered to the subject on the same day as administration of the vaccine to the subject.

12. The method of any one of claims 1-11, wherein the administration of the FcRn antagonist comprises administration of an initial dose of the FcRn antagonist and one or more subsequent doses of the FcRn antagonist.

13. The method of claim 12, wherein a subsequent dose of the FcRn antagonist is administered to the subject ≤4 weeks after administration of the vaccine to the subject.

14. The method of claim 12 or 13, wherein a subsequent dose of the FcRn antagonist is administered to the subject ≤2 weeks or ≤1 week after administration of the vaccine to the subject.

15. The method of any one of claims 12-14, wherein a subsequent dose of the FcRn antagonist is administered to the subject ≤24 hours after administration of the vaccine to the subject.

16. The method of any one of claims 12-15, wherein a subsequent dose of the FcRn antagonist is administered to the subject on the same day as administration of the vaccine to the subject.

17. The method of any one of claims 1-16, wherein the FcRn antagonist is administered to the subject at a fixed dose of 20 mg to 20,000 mg or at a dose of 0.2 mg/kg to 200 mg/kg.

18. The method of claim 17, wherein the FcRn antagonist is administered intravenously at a dose of 2 mg/kg to 200 mg/kg once weekly or once every 2 weeks.

19. The method of claim 17 or 18, wherein the FcRn antagonist is administered intravenously at a dose of 3 mg/kg to 60 mg/kg once weekly or once every 2 weeks.

20. The method of any one of claims 17-19, wherein the FcRn antagonist is administered intravenously at a dose of 10 mg/kg once weekly or once every 2 weeks.

21. The method of any one of claims 17-19, wherein the FcRn antagonist is administered intravenously at a dose of 25 mg/kg once weekly or once every 2 weeks.

22. The method of claim 17, wherein the FcRn antagonist is administered subcutaneously at a fixed dose of 200 mg to 20,000 mg once weekly, once every 2 weeks, once every 3 weeks, once every 4 weeks, once monthly, or once every 6 weeks.

23. The method of claim 22, wherein the FcRn antagonist is administered subcutaneously at a fixed dose of 750 to 3000 mg once weekly, once every 2 weeks, once every 3 weeks, once every 4 weeks, once monthly, or once every 6 weeks.

24. The method of claim 22 or 23, wherein the FcRn antagonist is administered subcutaneously at a fixed dose of 1000 mg or 2000 mg once weekly, once every 2 weeks, once every 3 weeks, once every 4 weeks, once monthly, or once every 6 weeks.

25. The method of any one of claims 22-24, wherein the FcRn antagonist is first administered subcutaneously at a fixed dose of 1000 mg twice on the same day.

26. The method of any one of claims 22-24, wherein the FcRn antagonist is administered subcutaneously at a fixed dose of 1000 mg once weekly.

27. The method of any one of claims 1-26, wherein the FcRn antagonist is administered for 52 weeks or less.

28. The method of any one of claims 1-27, wherein the FcRn antagonist is administered for 26 weeks or less.

29. The method of any one of claims 1-28, wherein the FcRn antagonist is administered for 4 weeks or less.

30. The method of any one of claims 1-28, wherein the FcRn antagonist is administered for at least 4 weeks.

31. The method of any one of claims 1-27, wherein the FcRn antagonist is administered for at least 26 weeks.

32. The method of any one of claims 1-26, wherein the FcRn antagonist is administered for at least 52 weeks.

33. The method of any one of claims 1-26, wherein the FcRn antagonist is administered using a phased dosing schedule comprising a first treatment cycle and one or more subsequent treatment cycles, wherein the first treatment cycle and the one or more subsequent treatment cycles each independently comprise administration of 1-5 doses of the FcRn antagonist within 1 month.

34. The method of claim 33, wherein the first treatment cycle comprises weekly administration of the FcRn antagonist for 4 weeks.

35. The method of claim 33 or 34, wherein the one or more subsequent treatment cycles each comprise weekly administration of the FcRn antagonist for 4 weeks.

36. The method of any one of claims 33-35, wherein each of the one or more subsequent treatment cycles is administered to the subject based on clinical evaluation.

37. The method of any one of claims 33-36, wherein each of the one or more subsequent treatment cycles is administered ≥50 days from the start of the previous treatment cycle.

38. The method of any one of claims 1-37, wherein the FcRn antagonist comprises two, three, or four FcRn binding regions.

39. The method of any one of claims 1-38, wherein the FcRn antagonist comprises or consists of a variant Fc region or FcRn binding fragment thereof.

40. The method of claim 39, wherein the variant Fc region or FcRn binding fragment thereof binds to FcRn with a higher affinity at pH 6.0 as compared to a corresponding wild-type Fc region.

41. The method of claim 39 or 40, wherein the variant Fc region or FcRn binding fragment thereof binds to FcRn with a higher affinity at pH 7.4 as compared to a corresponding wild-type Fc region.

42. The method of any one of claims 39-41, wherein the variant Fc region comprises or consists of a first Fc domain and a second Fc domain which form a homodimer or heterodimer.

43. The method of claim 42, wherein the first Fc domain and/or the second Fc domain comprise amino acids Y, T, E, K, and F at EU positions 252, 254, 256, 433, and 434, respectively.

44. The method of claim 42 or 43, wherein the first Fc domain and/or the second Fc domain comprise amino acids Y, T, E, K, F, and Y at EU positions 252, 254, 256, 433, 434, and 436, respectively.

45. The method of any one of claims 42-44, wherein the first Fc domain and/or the second Fc domain comprise an amino acid sequence independently selected from the group consisting of SEQ ID NO: 2, SEQ ID NO: 3, SEQ ID NO: 20, and SEQ ID NO: 21.

46. The method of any one of claims 42-45, wherein the first Fc domain and the second Fc domain comprise an amino acid sequence independently selected from the group consisting of SEQ ID NO: 2, SEQ ID NO: 3, SEQ ID NO: 20, and SEQ ID NO: 21.

47. The method of any one of claims 1-46, wherein the FcRn antagonist is efgartigimod.

48. The method of any one of claims 1-46, wherein the FcRn antagonist is an anti-FcRn antibody.

49. The method of any one of claims 1-37, wherein the FcRn antagonist is administered to the subject as part of a composition comprising a population of FcRn antagonist molecules, wherein at least a portion of the FcRn antagonist molecules in the population consist of a variant Fc region comprising a dimer of a first Fc domain and a second Fc domain, wherein the amino acid sequence of the first and second Fc domain consists of SEQ ID NO: 1, provided that the population is not a homogeneous population of homodimeric FcRn antagonist molecules in which the amino acid sequence of both the first and the second Fc domain consists of SEQ ID NO: 2, 3, 20, or 21, wherein the population comprises:
(a) a first subpopulation of FcRn antagonist molecules, wherein the amino acid sequences of both the first and the second Fc domains of the FcRn antagonist molecules in the first subpopulation consist of SEQ ID NO: 3; and
(b) at least one of:
(i) a second subpopulation of FcRn antagonist molecules, wherein the amino acid sequences of the first and the second Fc domains of the FcRn antagonist molecules in the second subpopulation consist of SEQ ID NO: 3 and SEQ ID NO: 12, respectively;
(ii) a third subpopulation of FcRn antagonist molecules, wherein the amino acid sequences of the first and the second Fc domains of the FcRn antagonist molecules in the third subpopulation consist of SEQ ID NO: 3 and SEQ ID NO: 9, respectively;
(iii) a fourth subpopulation of FcRn antagonist molecules, wherein the amino acid sequences of both the first and the second Fc domains of the FcRn antagonist molecules in the fourth subpopulation consist of SEQ ID NO: 3, and wherein two asparagine residues in each FcRn antagonist molecule in the fourth subpopulation are deaminated;
(iv) a fifth subpopulation of FcRn antagonist molecules, wherein the amino acid sequences of the first and the second Fc domains of the FcRn antagonist molecules in the fifth subpopulation consist of SEQ ID NO: 3, and wherein one asparagine residue in each FcRn antagonist molecule in the fifth subpopulation is deaminated;
(v) a sixth subpopulation of FcRn antagonist molecules, wherein the amino acid sequences of the first and the second Fc domains of the FcRn antagonist molecules in the sixth subpopulation consist of SEQ ID NO: 2 and SEQ ID NO: 3, respectively;
(vi) a seventh subpopulation of FcRn antagonist molecules, wherein the amino acid sequences of the first and the second Fc domains of the FcRn antagonist molecules in the seventh subpopulation consist of SEQ ID NO: 3, and wherein one methionine residue or one tryptophan residue in each FcRn antagonist molecule in the seventh subpopulation is oxidized;
(vii) an eighth subpopulation of FcRn antagonist molecules, wherein the amino acid sequences of both the first and the second Fc domains of the FcRn antagonist molecules in the eighth subpopulation consist of SEQ ID NO: 2;
(viii) a ninth subpopulation of FcRn antagonist molecules, wherein the amino acid sequences of the first and the second Fc domains of the FcRn antagonist molecules in the ninth subpopulation consist of SEQ ID NO: 3 and SEQ ID NO: 6, respectively;
(ix) a tenth subpopulation of FcRn antagonist molecules, wherein the amino acid sequences of the first and the second Fc domains of the FcRn antagonist molecules in the tenth subpopulation consist of SEQ ID NO: 2 and SEQ ID NO: 3, respectively, and wherein one methionine residue or one tryptophan residue in each FcRn antagonist molecule in the tenth subpopulation is oxidized; and
(x) an eleventh subpopulation of FcRn antagonist molecules, wherein the amino acid sequences of both the first and the second Fc domains of the FcRn antagonist molecules in the eleventh subpopulation consist of SEQ ID NO: 3, and wherein two amino acid residues, independently selected from a methionine residue and a tryptophan, in each FcRn antagonist molecule in the eleventh subpopulation are oxidized.

50. The method of any one of claims 1-49, wherein the vaccine is administered to the subject in a single dose.

51. The method of any one of claims 1-49, wherein the vaccine is administered to the subject in two or more doses.

52. The method of claim 51, wherein the vaccine is administered to the subject in two doses.

53. The method of claim 51 or 52, wherein the doses are administered to the subject more than 1 week apart.

54. The method of any one of claims 51-53, wherein the doses are administered to the subject more than 2 weeks apart.

55. The method of any one of claims 51-54, wherein the doses are administered to the subject less than 3 months apart.

56. The method of any one of claims 51-55, wherein the doses are administered to the subject less than 2 months or less than 1 month apart.

57. The method of any one of claims 1-56, wherein the vaccine is selected from a recombinant vaccine, a toxoid vaccine, an inactivated vaccine, and a live attenuated vaccine.

58. The method of any one of claims 1-56, wherein the vaccine is a recombinant vaccine, a toxoid vaccine, or an inactivated vaccine.

59. The method of claim 57 or 58, wherein the recombinant vaccine is selected from an mRNA vaccine, a viral vector vaccine, and a subunit vaccine.

60. The method of claim 59, wherein the subunit vaccine is selected from a polysaccharide vaccine, a conjugate vaccine, and a protein-based vaccine.

61. The method of any one of claims 1-60, wherein the vaccine is a T-cell dependent vaccine.

62. The method of any one of claims 1-61, wherein the vaccine is a COVID-19 vaccine, an influenza vaccine, or a pneumococcal vaccine.

63. The method of claim 62, wherein the COVID-19 vaccine is selected from tozinameran, elasomeran, NVX-CoV2373, Ad26.COV2.S, AZD1222, CoronaVac, BBV152, AD5-nCOV, MVC-COV1901, BBIBP-CorV, and Gam-COVID-Vac.

64. The method of claim 62, wherein the influenza vaccine is selected from Audenz, Fluad Quadrivalent, Fluad, Afluria Quadrivalent, Afluria Quadrivalent Southern Hemisphere, Flucelvax Quadrivalent, Flulaval Quadrivalent, Afluria, Afluria Southern Hemisphere, FluLaval, FluMist, Fluarix, Fluvirin, Agriflu, Fluzone, Fluzone High-Dose, Fluzone Intradermal, Flucelvax, Flublok, Flublok Quadrivalent, FluMist Quadrivalent, Fluarix Quadrivalent, and Fluzone Quadrivalent.

65. The method of claim 62, wherein the pneumococcal vaccine is selected from Pneumovax 23, Prevnar 13, VAXNEUVANCE™, and Prevnar 20.

66. The method of any one of claims 1-65, wherein administration of the vaccine to the subject elicits a protective immune response.

67. The method of any one of claims 1-66, wherein the vaccine comprises one or more pathogenic antigens, and wherein IgG antibody titers against one or more of the pathogenic antigens are maintained at or above a protective threshold following administration of the FcRn antagonist to the subject.

68. The method of claim 67, wherein the vaccine is a pneumococcal conjugate vaccine, and wherein the protective threshold is an IgG antibody concentration of ≥350 ng/mL.

69. The method of claim 68, wherein the pneumococcal conjugate vaccine is a 13-valent pneumococcal conjugate vaccine (PCV13).

70. The method of claim 67, wherein the vaccine is an influenza vaccine, and wherein the protective threshold is a neutralizing titer of ≥1:40, optionally measured using a hemagglutination inhibition assay.

71. The method of claim 67, wherein the vaccine is a COVID-19 vaccine, and wherein the protective threshold is an anti-SARS-CoV-2 Spike protein receptor-binding domain (S-RBD) IgG level of ≥620.2 AU/mL.

72. The method of claim 67, wherein the vaccine is a COVID-19 vaccine, and wherein the protective threshold is an anti-S-RBD IgG level of ≥82.82 AU/mL.

73. The method of any one of claims 1-72, wherein the vaccine comprises one or more pathogenic antigens, and wherein IgG antibody titers against one or more of the pathogenic antigens are maintained at or above a baseline value following administration of the FcRn antagonist to the subject.

74. The method of claim 73, wherein the baseline value is an IgG antibody titer against the one or more pathogenic antigens in the subject before administration of the FcRn antagonist to the subject.

75. The method of any one of claims 1-74, wherein the subject has an antigen-specific IgG response following subsequent antigen challenge.

76. The method of any one of claims 1-75, wherein the subject shows at least a 4-fold increase in antigen-specific IgG following a second administration of the vaccine.

77. The method of any one of claims 1-76, wherein the subject shows at least a 20-fold increase in antigen-specific IgG following a second administration of the vaccine.

78. The method of claim 76 or 77, wherein the antigen-specific IgG is an anti-S-RBD IgG.

79. The method of any one of claims 76-78, wherein the vaccine comprises one or more SARS-CoV-2 antigens selected from the group consisting of spike protein S2, nucleocapsid, spike protein S1, full-length spike protein, and spike protein receptor-binding domain.

80. The method of any one of claims 1-61, wherein the vaccine is not a seasonal vaccine.

81. The method of any one of claims 1-80, wherein the antibody-mediated disorder is an IgG-mediated disorder.

82. The method of any one of claims 1-81, wherein the antibody-mediated disorder is an autoimmune disease.

83. The method of claim 82, wherein the autoimmune disease is selected from the group consisting of: allogenic islet graft rejection, alopecia areata, ankylosing spondylitis, antiphospholipid syndrome, autoimmune Addison's disease, Alzheimer's disease, antineutrophil cytoplasmic autoantibodies (ANCA), autoimmune diseases of the adrenal gland, autoimmune hemolytic anemia, autoimmune hepatitis, autoimmune myocarditis, autoimmune neutropenia, autoimmune oophoritis and orchitis, immune thrombocytopenia (ITP or idiopathic thrombocytopenic purpura, idiopathic thrombocytopenia purpura, immune mediated thrombocytopenia, or primary immune thrombocytopenia), autoimmune urticaria, Behcet's disease, bullous pemphigoid (BP), cardiomyopathy, Castleman disease, celiac sprue-dermatitis, chronic fatigue immune dysfunction syndrome, chronic inflammatory demyelinating polyneuropathy (CIDP), Churg-Strauss syndrome, cicatricial pemphigoid, CREST syndrome, cold agglutinin disease, Crohn's disease, dilated cardiomyopathy, discoid lupus, epidermolysis bullosa acquisita, essential mixed cryoglobulinemia, factor VIII deficiency, fibromyalgia-fibromyositis, glomerulonephritis, Goodpasture syndrome, Graves' disease, Guillain-Barre syndrome, graft-versus-host disease (GVHD), Hashimoto's thyroiditis, hemophilia A, idiopathic inflammatory myopathies (IIMs), idiopathic membranous neuropathy, idiopathic pulmonary fibrosis, IgA neuropathy, IgM polyneuropathies, immune-mediated necrotizing myopathy (IMNM), juvenile arthritis, Kawasaki disease, lichen planus, lichen sclerosus, lupus erythematosus, lupus nephritis, Ménière's disease, mixed connective tissue disease, mucous membrane pemphigoid, multiple sclerosis, Type 1 diabetes mellitus, multifocal motor neuropathy (MMN), myasthenia gravis (MG), generalized myasthenia gravis (gMG), myositis, paraneoplastic bullous pemphigoid, pemphigoid gestationis, Pemphigus vulgaris (PV), Pemphigus foliaceus (PF), pernicious anemia, polyarteritis nodosa, polychondritis, polyglandular syndromes, polymyalgia rheumatica, polymyositis, dermatomyositis (DM), necrotizing autoimmune myopathy (NAM), AntiSynthetase Syndrome (ASyS), primary agammaglobulinemia, primary biliary cirrhosis, psoriasis, psoriatic arthritis, relapsing polychondritis, Raynaud's phenomenon, Reiter's syndrome, rheumatoid arthritis, sarcoidosis, scleroderma, Sjögren's syndrome, solid organ transplant rejection, stiff-person syndrome, systemic lupus erythematosus, Takayasu's arteritis, toxic epidermal necrolysis (TEN), Stevens-Johnson syndrome (SJS), temporal arteritis/giant cell arteritis, thrombotic thrombocytopenia purpura, ulcerative colitis, uveitis, dermatitis herpetiformis vasculitis, anti-neutrophil cytoplasmic antibody-associated vasculitides, vitiligo, and Wegener's granulomatosis.

84. The method of claim 82 or 83, wherein the autoimmune disease is gMG, PV, or PF.

85. The method of claim 82, wherein the autoimmune disease is not gMG, PV, or PF.

86. The method of claim 82, wherein the autoimmune disease is selected from the group consisting of: allogenic islet graft rejection, alopecia areata, ankylosing spondylitis, antiphospholipid syndrome, autoimmune Addison's disease, Alzheimer's disease, ANCA, autoimmune diseases of the adrenal gland, autoimmune hemolytic anemia, autoimmune hepatitis, autoimmune myocarditis, autoimmune neutropenia, autoimmune oophoritis and orchitis, immune thrombocytopenia (ITP or idiopathic thrombocytopenic purpura, idiopathic thrombocytopenia purpura, immune mediated thrombocytopenia, or primary immune thrombocytopenia), autoimmune urticaria, Behcet's disease, BP, cardiomyopathy, Castleman disease, celiac sprue-dermatitis, chronic fatigue immune dysfunction syndrome, CIDP, Churg-Strauss syndrome, cicatricial pemphigoid, CREST syndrome, cold agglutinin disease, Crohn's disease, dilated cardiomyopathy, discoid lupus, epidermolysis bullosa acquisita, essential mixed cryoglobulinemia, factor VIII deficiency, fibromyalgia-fibromyositis, glomerulonephritis, Goodpasture syndrome, Graves' disease, Guillain-Barre syndrome, GVHD, Hashimoto's thyroiditis, hemophilia A, IIMs, idiopathic membranous neuropathy, idiopathic pulmonary fibrosis, IgA neuropathy, IgM polyneuropathies, IMNM, juvenile arthritis, Kawasaki disease, lichen planus, lichen sclerosus, lupus erythematosus, lupus nephritis, Ménière's disease, mixed connective tissue disease, mucous membrane pemphigoid, multiple sclerosis, Type 1 diabetes mellitus, MMN, myositis, paraneoplastic bullous pemphigoid, pemphigoid gestationis, pernicious anemia, polyarteritis nodosa, polychondritis, polyglandular syndromes, polymyalgia rheumatica, polymyositis, DM, NAM, ASyS, primary agammaglobulinemia, primary biliary cirrhosis, psoriasis, psoriatic arthritis, relapsing polychondritis, Raynaud's phenomenon, Reiter's syndrome, rheumatoid arthritis, sarcoidosis, scleroderma, Sjögren's syndrome, solid organ transplant rejection, stiff-person syndrome, systemic lupus erythematosus, Takayasu's arteritis, TEN, SJS, temporal arteritis/giant cell arteritis, thrombotic thrombocytopenia purpura, ulcerative colitis, uveitis, dermatitis herpetiformis vasculitis, anti-neutrophil cytoplasmic antibody-associated vasculitides, vitiligo, and Wegener's granulomatosis.

87. An FcRn antagonist for use in the treatment of an antibody-mediated disorder, wherein the treatment is performed according to the method of any one of claims 1-86.

88. An FcRn antagonist for use in the manufacture of a medicament for the treatment of an antibody-mediated disorder, wherein the treatment is performed according to the method of any one of claims 1-86.

89. Use of an FcRn antagonist in the treatment of an antibody-mediated disorder, wherein the treatment is performed according to the method of any one of claims 1-86.

90. Use of an FcRn antagonist in the manufacture of a medicament for the treatment of an antibody-mediated disorder, wherein the treatment is performed according to the method of any one of claims 1-86.

91. A vaccine for use in the vaccination of a subject having an antibody-mediated disorder, wherein the vaccination is performed according to the method of any one of claims 1-86.

92. Use of a vaccine in the vaccination of a subject having an antibody-mediated disorder, wherein the vaccination is performed according to the method of any one of claims 1-86.
